SEQUENCE LISTING (119 sequences)

SEQ ID NO: 1 - protein, 531 aa, synthetic construct
MIINNCVTPKDTQPAPLPSETDIVSNDINIKNELSLKHLELLEEVRYLLKNYSSEPLDMVDALQKLCINHYYEDEIGLILKLLYARMSNNGDYKHDKSLYEVSLSFRILRQEGYYVSADVFADFKQKDGKFKEEIAQDVKGLMALYEASQLSLDGERILEEASDFSSGALKDMVPSLDLHQAMIATNTLQHTYQRTSSTFMVKKFMKVYTGTPMCELAQLELTTKVQSLHRTELEQISRWWKDLGLAQELKLARNQPLHWYLWPMASLTDLSLSEQRIELTKPIAFIFLIDDIFDVYGTLDQLVILTQAVNRWESNSLERLPHHLRICIQALFDVTNEISDNIYKKHGFNPIDFLKQSWINLCEAFLVEAKWFAEGYMPTAEDYLNNGMVSTGAHVVIVHMFFLLGGGPNIKSASVVNENQGITSCLAKILRLWDDLGSAEDVDQDGNDGSYVTYYMKENAGCSIQKAHEHVMEMISNTWKQLNAECLYSSHLSRTFTKACLNLARMIPMMYDYDENHSLPFIKEYINSMF

SEQ ID NO: 2 - protein, 571 aa, synthetic construct
MAPPPSSYPPSARNNFTQVGTGLSDTMNEPSTQKWSITSHDGTLVSSPIILHNSKANNACYTDEFHVDHESKLNEVRDLLNKVGEDYQLEGLVMIDAIQCLNIDYRFRKEIESILQSQDETSRVHENDEHDLYQTALRFRLLRQEGHFVSAEVFDKFKNKEGKFKQELVEDIRGMMGLYEAAQLRIEGEHILDEAESFCRRLFKGCMPYLNSHEAKLVESTLKHPYRKSLSRIGAKNFANNFQGNFKWITVLEELSNLDLKIVQSIHQKELQQISHWWEELGLAKELKLARDQPLKWHMWSMEVLQDPSLSEQRIELTKPISLVYIIDDIFDVYGTLDELTLFTEAVTRWEFTDANQLPNYMRTCFQTLFDITYEIGYKFFIVHGWNPIDTLRKSWVTLCKAFLAEAKWFASGELPKADKYLKNGIISSGVHVVLVHMFFLLGQDLTKECLDLVNDNNNIPGIINYTATILRLWDDLGSAKDENQDGYDGSYLECFMNEHKGLLMKTAREHMIGMISDAWKCLNKECLSPNPFSSSFTKASLNAARMVPLMYNYDDNHCLPDLEEHIQCLF

SEQ ID NO: 3 - protein, 528 aa, synthetic construct
MTISVTRKDTQPTPFPSETDIVSNDINIKNELSIRHLELLEEVRNLLKNCSSKPLVMVDTLQKLCINHHYEEEIGLILKSLYTRMSNNDDYKHDKSLYEVSLSFRILRQHGYYVSADVFANFKQKDGKFKGEITQDLKGLIALYEASQLSLEGEQILKEASDFSSGALKEMMPSLDQDQAMIVTNTLQHTYQRTTSTFMVKKFMKVYTGTPMCELAQLELTKVQSLHRTEVDQISRWWKGLGLAQELKLARNQPLHWYLWPMASLTDLSLSEQRVELTKPIAFIFLIDDIFDVYGTLDQLVILTQAVNRWESNRLEQLPYHLRICIQALFDVTNEISDKIYKKHGFNPIEFLKQSWINLCDAFLVEAKWFAEGYMPTAEDYLNNGMVSTGVHVVIVHMFFLLGGGPNIKSASVVNENQGIMSCLAKILRLWDDLGSAKDVDQDGNDGSYVTYYMKENAGCSIQKAHEHVMEMISNTWKQLNAECLYSSHFSRTFTKACLNLAKMIPMMYDYDENHSLPFIEEYINSMF

SEQ ID NO: 4 - protein, 525 aa, synthetic construct
MSINILHGDLPDVKVMSPQADATEKIDELKEKIRRVLMTTSDPKMSLKLVDTIQRLRIGYYFQEDINEILEKLKQCLPDDELHIVALCFRLLRQNGIPTNSEVFRKFIDMNGEFIKSTSEDIEGLLSLYEASYMGSNEEIFLVHAKKITTRELNICVPKLSPKLSKKVLQALELPMHLRMETLEARRYIEDYGNEEDHNPLLLELAKLDYNHVQSLFRRELVEMARWWNHLGIARKFSFVRDRHVECFLWTVGVLPEPKYSATRIVMAKITSILLLLDDIYDTYGSYDDLVLLTKIIQRWDMTEIEQLPEYMQACYMALYNTTSEICDKVLRERGLYVEQFLRKTWIKIVEGYMVEVKWLKTGTIPNFKEYMDNAVTTSGSYMAFVHMFFLICDEVNKENMADLLEPYPKFFTLAGTILRLWDDLGTVKEEQERGEVLSSIQLLMKEKKITCDKDERKQILELIHELWKDLNAELVAPNAVLWPMIRVALNMSRTSQVVYQHNEDSYLSSVKDHVKNLFFKAIDM

SEQ ID NO: 5 - DNA, 1596 bp, synthetic construct
  source 1..1596: mol_type = other DNA
atgatcatcaacaattgtgttacaccgaaagacacccagccagcccctctcccatctgaaacagatattgtgtcaaatgacattaacattaagaatgaattgtccctcaaacatctagaattgttagaagaagtccgatatcttctaaagaattattcttccgaacctttggacatggttgacgctcttcaaaaactttgcatcaaccattactatgaagacgagattggcttgattttgaaattgctctacgctagaatgtcgaataatggcgactacaagcatgataaaagtctttatgaggtttccctttcttttcgcatccttcgtcaagaaggttactatgtctcagctgatgtttttgctgatttcaaacaaaaggatgggaagtttaaagaagaaatagcacaagatgttaagggtctaatggcgttatatgaagcttcacagctaagcctggatggagaacgaatacttgaagaggcttcagattttagcagtggtgctcttaaagacatggtgccttctttagatctacatcaagctatgattgctactaacacattacaacatacgtatcaaagaacctcatcaactttcatggttaaaaaattcatgaaagtatacacgggtacacccatgtgtgaactagcccaattagagttgactactaaagtccagtcactacatcgaacagaactagaacaaatctccagatggtggaaggacttggggttggcgcaagagttgaagcttgcaagaaaccaaccgttgcactggtacttatggccaatggctagcctcacagatctcagcttgtccgagcagagaattgagctcacgaagcccattgctttcatctttctaattgatgacatttttgatgtttatggaactctagaccaacttgttatcttgacacaagcagtgaatagatgggaaagtaacagccttgaacgactcccacatcacctaaggatctgtattcaagctctatttgacgtaacaaacgaaataagtgacaatatctacaagaagcatgggttcaatcccattgattttttgaaacaatcgtggataaacctttgtgaagcttttctagtagaggcgaaatggtttgctgagggatacatgccgacggcagaggattacttgaataatgggatggttagtacaggggcacatgtcgtgatagtgcacatgttcttcctcctcggtggtggccctaacataaaaagtgctagcgttgtaaacgaaaaccaagggattacgtcttgtttggcaaagattcttcgtctttgggatgacttaggaagtgctgaggatgtggatcaagacggtaatgatggatcatatgtgacatattacatgaaagagaatgcaggttgttctattcaaaaggcacatgaacatgttatggaaatgatatcgaatacttggaagcaactaaatgcggagtgcctttactcaagtcatttatcacgtacattcaccaaagcttgcctcaatcttgcaagaatgattccaatgatgtacgattatgatgagaatcattcccttcctttcatcaaggaatatataaactccatgttttaa

SEQ ID NO: 6 - DNA, 1716 bp, synthetic construct
  source 1..1716: mol_type = other DNA
atggcaccccctccttcctcctatcctccaagtgctcgaaacaattttacacaagttggcacaggacttagtgatacgatgaacgagccttctactcagaaatggagcattaccagccatgatggcaccttagtttcaagccctataatacttcacaactctaaagcaaacaatgcttgttatactgatgaatttcatgtcgatcatgaaagcaaattgaatgaagttagggatttgcttaacaaagttggagaagattatcaattagaaggtttggtcatgatcgacgccattcaatgcctaaacattgactatcgcttccgaaaggagattgagtccatcctacaaagtcaggatgagacatctagggttcatgaaaatgatgaacacgatctttaccagactgcacttcgttttcgactgctgagacaagaaggccattttgtgtctgcagaagtgtttgacaaattcaagaacaaagaggggaagtttaagcaagaactagttgaagatataaggggaatgatgggcttatatgaagctgcgcagcttagaattgaaggagaacatatacttgatgaagccgaaagcttttgtcgtcggctctttaaaggatgcatgccctatcttaactctcatgaagcaaaacttgtcgagagcacgctaaagcatccctatcgaaagagcttgtcacgcataggggccaaaaacttcgctaacaatttccaaggcaattttaaatggattactgttcttgaagaactatcaaatttagatttgaaaatagttcaatctatacaccagaaggaactacaacaaatttcccattggtgggaagaacttggtttagcaaaggaattgaagctcgcaagagaccaaccgctaaaatggcacatgtggtccatggaagtgttacaagatccaagcttgtctgagcaaaggattgagcttacaaagcccatttctcttgtatacataattgatgacattttcgacgtttatgggacgcttgatgaactcactctcttcacggaagcagtcactagatgggaattcaccgacgcaaatcaactgcctaactacatgaggacatgtttccagactctctttgatatcacttatgaaattggctacaagtttttcatagttcatggttggaaccctatagacacactacgaaaatcgtgggttactttgtgcaaagcatttttggcggaagcaaaatggtttgcttctggggaattgccaaaggcagataaatacttgaaaaatggaattattagttcaggagtgcatgtagtacttgttcacatgttttttctcttgggtcaagatttaacaaaggaatgtttagacctagtgaacgacaacaacaatattccaggcatcataaactatacggcaacgattcttcgtctttgggatgacttgggaagtgcaaaggatgagaatcaagatgggtacgacggatcatatctggaatgtttcatgaatgaacacaaaggtttattaatgaagactgcaagagagcatatgattggcatgatttcggatgcatggaagtgcttgaacaaggaatgcctctctccaaatccattttcatcatctttcacaaaggcttctcttaatgctgcaaggatggttccattgatgtacaattacgacgacaaccattgtctcccagatcttgaggagcatatacaatgtttgttttaa

SEQ ID NO: 7 - DNA, 1587 bp, synthetic construct
  source 1..1587: mol_type = other DNA
atgacaatctctgttactagaaaggatactcaaccaacaccatttccatctgaaactgatattgtttctaacgatatcaacatcaagaacgaattgtctattagacatttggaattgttggaagaagttagaaatttgttgaagaactgttcttctaagccattagttatggttgatactttacaaaagctgtgtattaaccatcattacgaagaagaaatcggtttgattttgaaatctttgtacacaagaatgtccaataacgatgattataagcatgataagtccttatacgaagtttctttgtcttttagaatcttgagacaacatggttattacgtttctgctgatgtttttgctaattttaagcaaaaggacggtaaatttaagggtgaaattacacaagatctgaaaggtttaatcgctttatatgaagctagtcaattgtctttggaaggtgaacaaattttaaaggaagctagtgatttctcttctggtgctttgaaagaaatgatgccatctttggatcaagatcaagctatgattgttacaaatactttgcaacatacctaccaaagaacaacttctacttttatggttaagaagttcatgaaggtttacactggtacaccaatgtgtgaattggctcaattggaattgactaaagttcaatctttgcatagaacagaagttgatcaaatttctagatggtggaaaggtttaggtttggctcaagaattaaaattagctagaaaccaaccattgcattggtatttatggccaatggcttctttgactgatttgtctttgtctgaacaaagagttgaattgactaagccaattgcttttattttcttgatcgatgatatcttcgacgtttatggtactttggatcaattagttatcttgactcaagctgttaatagatgggaatctaatagattagagcaattaccataccatttgagaatttgtatccaagctctatttgatgttacaaatgaaatctctgacaagatttacaagaagcatggttttaatccaatcgaatttttgaagcaatcctggattaatttgtgtgatgcttttttggttgaggctaaatggtttgctgaaggttatatgccaactgctgaagattatttaaacaatggtatggtttccacaggtgttcatgttgttattgttcatatgtttttcctgttgggtggtggtccaaatattaaatctgcttctgttgttaacgagaatcaaggtattatgtcttgtttggctaaaattttgaggttatgggatgatttaggttctgctaaagatgttgatcaagatggtaatgatggttcttatgttacatattacatgaaggaaaacgctggttgttctattcaaaaagctcatgaacatgttatggaaatgatttctaacacttggaaacaattgaacgctgaatgtttatattcctctcatttttctaggacattcactaaagcatgtttgaatttggctaaaatgatcccaatgatgtatgattatgacgaaaatcattccttgccatttattgaagaatacattaactccatgttctaa

SEQ ID NO: 8 - DNA, 1578 bp, synthetic construct
  source 1..1578: mol_type = other DNA
atgtctatcaacatcttgcatggtgatttgccagatgttaaagttatgtctccacaagctgatgctacagaaaaaattgatgaattaaaggagaagatcaggagagttttgatgactacatctgatccaaaaatgtctttaaagttggttgatactatccaaagattgagaattggttattacttccaagaagatatcaacgaaatcttggaaaaattgaagcaatgtttgccagatgatgaattgcatattgttgctttgtgttttagattgttgagacaaaatggtatcccaacaaattctgaagtttttagaaagttcatcgacatgaatggtgaatttattaagtctacttccgaagatatcgaaggtttgttatctttgtatgaagctagttatatgggttctaatgaagaaattttcctggttcatgctaaaaaaatcacaactagagaattgaacatctgtgttccaaaattgtctccaaaattgtcaaaaaaggtcttgcaagctctagaattgccaatgcatttgagaatggaaacattggaagctagaagatatattgaagattacggtaatgaggaagatcataatccattattactggaattggctaaattggattacaatcatgttcaatctctgtttagaagagaattagttgaaatggctagatggtggaatcatttgggtattgctagaaaattttcctttgttagagacagacatgttgaatgttttttgtggactgttggtgttttgccagaaccaaaatattctgctactagaattgttatggctaaaattacttccatcttgttattgttggacgatatttatgacacttacggttcttatgatgatttggttttattgacaaagatcatccaaagatgggatatgacagaaattgaacaattgccagaatatatgcaagcatgttatatggctttgtataatactacatccgaaatttgtgacaaggttttaagagaaagaggtttgtatgttgaacaatttttgagaaagacctggattaaaatcgttgaaggttatatggttgaggttaaatggttaaaaaccggtactattccaaattttaaggaatatatggacaacgctgttactacttctggttcttatatggcttttgttcatatgtttttcctgatttgtgacgaagttaataaggaaaatatggctgatttgttggaaccatatccaaaatttttcactttggctggtacaattttgagattgtgggatgatttaggtactgttaaagaagaacaagaaagaggtgaagttttgtcttctattcaattattgatgaaggagaagaagatcacttgtgataaagatgaaagaaagcaaatcttggaattgattcatgaattgtggaaagatttgaacgctgaattagttgctccaaatgctgttttgtggccaatgattagagttgctttaaatatgtctaggacatctcaagttgtttatcaacataatgaggattcttacttgtcttctgttaaagatcatgttaagaacttgttcttcaaggctattgatatgtaa

SEQ ID NO: 9 - DNA, 1734 bp, synthetic construct
  source 1..1734: mol_type = other DNA
atggcaccccctccttcctcctatcctccaagtgctcgaaacaattttacacaagttggcacaggacttggtgatacgatgaacgagccttctactcagaaatggagcattaccagccatgatggcaccttagtttcaagccctataatacttcacaactctaaagcaaacaatgcttgttatactgatgaatttcatgtcgatcatgaaagaaaattgaatgaagttagggatttgcttaacaaagttggagaagattatcaattagaaggtttggtcatgatcgacgccattcaatgcctaaacattgactatcacttccgaaaggagattgagtccatcctacaatgtcaggatgagacatctagggttcatgaaaatgatgaacacgatctttaccagactgcacttcgttttcgactgctgagacaagaaggccattttgtgtctgcagaagtgtttgacaaattcaagaacaaagaggggaagtttaagcaagaactagttgaagatataaggggaatgatgggcttatatgaagctgcgcagcttagaattgaaggagaacatatacttgatgaagccgaaagcttttgtcgtcggctctttaaagcatgcatgccctatcttaactctcatgaagcaagacttgtcgagagcatgataaagcatccctatcgaaagagcttgtcacgcataggggccaaaaacttcgctaacaatttccaaggcaatttcaaatggattactgttcttgaagaactatcaaatttagatttgaaaatagttcaatctatacaccagaaggaactacaacaaatttcccattggtgggaagaactgggtttagcaaaggaattgaagctcgcaagagaccaaccgctaaaatggcacatgtggtccatggaagtgttaacagatccaagcttgtctgagcaaaggattgagcttacaaagcccatttctcttgtatacataattgatgacattttcgacgtttatgggacgcttgatgaactcactctcttcacggaagcagtcacaagatgggaattcaccgacgtaaatcaactgcctaactacatgaggacatgtttccagactctctttgatatcacttatgaaattggctataagtttttcatagttcatggttggaaccctataaacacactacgaaaatcgtgggttactttgtgcaaagcatttttggcggaagcaaaatggtttgcttctggggaattgccaaaggcagataaatacttgaaaaatgggattattagttcaggagtgcatgtagtacttgttcacatgttttttctcttgggtcaagatttaacaaaggaatgtttagacctagtgaacgacaacaacaatattccaggcatcataaactatacggcaacgattcttcgtctttgggatgacttgggaagtgcaaaggatgagaatcaagacgggtacgacggatcatatctggaatgtttcatgaatgaacacaaaggtttattaatgaagactgcaagagagcatatgattggcatgatttcggatgcatggaagtgcttgaacaaggaatgcctctctccaaatccattttcatcatctttcacaaaggcttctcttaatgctgcaaggatggttcctttgatgtacaattacgacgacaaccattgtctcccagatcttgaggaacatgcaatgtttgttttaaatatgataaacgttacatag

SEQ ID NO: 10 - DNA, 1734 bp, synthetic construct
  source 1..1734: mol_type = other DNA
atggcaccccctccttcctcctatcctccaagtgctcgaaacaattttacacaagttggcacaggacttggtgatacgatgaacgagccttctactcagaaatggagcattaccagccatgatggcaccttagtttcaagccctataatacttcacaactctaaagcaaacaatgcttgttatactgatgaatttcatgtcgatcatgaaagaaaattgaatgaagttagggatttgcttaacaaagttggagaagattatcaattagaaggtttggtcatgatcgacgccattcaatgcctaaacattgactatcacttccgaaaggagattgagtccatcctacaatgtcaggatgagacatctagggttcatgaaaatgatgaacacgatctttaccagactgcacttcgttttcgactgctgagacaagaaggccattttgtgtctgcagaagtgtttgacaaattcaagaacaaagaggggaagttcaagcaagaactagttgaagatataaggggaatgatgggcttatatgaagctgcgcagcttagaattgaaggagaacatatacttgatgaagccgaaagcttttgtcgtcggctctttaaagcatgcatgccctatcttaactctcatgaagcaagacttgtcgagagcatgataaagcatccctatcgaaagagcttgtcccgcataggggccaaaaacttcactaacaatttccaaggcaatttcaaatggattactgttcttgaagaactatcaaatttagatttgaaaatagttcaatctatacaccagaaggaactacaacaaatttcccattggtgggaagaactgggtttagcaaaggaattgaagctcgcaagagaccaaccgctaaaatggcacatgtggtccatggaagtgttaacagatccaagcttgtctgagcaaaggattgagcttacaaagcccatttctcttgtatacataattgatgacattttcgacgtttatgggacgcttgatgaactcactctcttcacggaagcagtcacaagatgggaattcaccgacgtaaatcaactgcctaactacatgaggacatgtttccagactctctttgatatcacttatgaaattggctacaagtttttcatagttcatggttggaaccctatagacacactacgaaaatcgtgggttactttgtgcaaagcatttttggcggaagcaaaatggtttgcttctggggaattgccaaaggcagataaatacttgaaaaatgggattattagttcaggagtgcatgtagtacttgttcacatgttttttctcttgggtcaagatttatcaaaggaatgtttagacctagtgaacgacaacaacaatattccaggcatcataaactatacggcaacgattcttcgtctttgggatgacttgggaagtgcaaaggatgagaatcaagacgggtacgacggatcatatctggaatgtttcatgaatgaacacaaaggtttattaatgaagactgcaagagagcatatgattggcatgatttcggatgcatggaagtgcttgaacaaggaatgcctctctccaaatccattttcatcatctttcacaaaggcttctcttaatgctgcaaggatggttcctttgatgtacaattacgacgacaaccattgtctcccagatcttgaggaacatgcaatgtttgttttaaatatgataaacgttacatag

SEQ ID NO: 11 - protein, 577 aa, synthetic construct
MAPPPSSYPPSARNNFTQVGTGLGDTMNEPSTQKWSITSHDGTLVSSPIILHNSKANNACYTDEFHVDHERKLNEVRDLLNKVGEDYQLEGLVMIDAIQCLNIDYHFRKEIESILQCQDETSRVHENDEHDLYQTALRFRLLRQEGHFVSAEVFDKFKNKEGKFKQELVEDIRGMMGLYEAAQLRIEGEHILDEAESFCRRLFKACMPYLNSHEARLVESMIKHPYRKSLSRIGAKNFANNFQGNFKWITVLEELSNLDLKIVQSIHQKELQQISHWWEELGLAKELKLARDQPLKWHMWSMEVLTDPSLSEQRIELTKPISLVYIIDDIFDVYGTLDELTLFTEAVTRWEFTDVNQLPNYMRTCFQTLFDITYEIGYKFFIVHGWNPINTLRKSWVTLCKAFLAEAKWFASGELPKADKYLKNGIISSGVHVVLVHMFFLLGQDLTKECLDLVNDNNNIPGIINYTATILRLWDDLGSAKDENQDGYDGSYLECFMNEHKGLLMKTAREHMIGMISDAWKCLNKECLSPNPFSSSFTKASLNAARMVPLMYNYDDNHCLPDLEEHAMFVLNMINVT

SEQ ID NO: 12 - protein, 577 aa, synthetic construct
MAPPPSSYPPSARNNFTQVGTGLGDTMNEPSTQKWSITSHDGTLVSSPIILHNSKANNACYTDEFHVDHERKLNEVRDLLNKVGEDYQLEGLVMIDAIQCLNIDYHFRKEIESILQCQDETSRVHENDEHDLYQTALRFRLLRQEGHFVSAEVFDKFKNKEGKFKQELVEDIRGMMGLYEAAQLRIEGEHILDEAESFCRRLFKACMPYLNSHEARLVESMIKHPYRKSLSRIGAKNFTNNFQGNFKWITVLEELSNLDLKIVQSIHQKELQQISHWWEELGLAKELKLARDQPLKWHMWSMEVLTDPSLSEQRIELTKPISLVYIIDDIFDVYGTLDELTLFTEAVTRWEFTDVNQLPNYMRTCFQTLFDITYEIGYKFFIVHGWNPIDTLRKSWVTLCKAFLAEAKWFASGELPKADKYLKNGIISSGVHVVLVHMFFLLGQDLSKECLDLVNDNNNIPGIINYTATILRLWDDLGSAKDENQDGYDGSYLECFMNEHKGLLMKTAREHMIGMISDAWKCLNKECLSPNPFSSSFTKASLNAARMVPLMYNYDDNHCLPDLEEHAMFVLNMINVT

SEQ ID NO: 13 - DNA, 59 bp, synthetic construct
  source 1..59: mol_type = other DNA
actaaagggaacaaaagctggagctctagtagtttaaacataacgagaacacacagggg

SEQ ID NO: 14 - DNA, 53 bp, synthetic construct
  source 1..53: mol_type = other DNA
cattaaagtaacttaaggagttaaatttaagcaaggattttcttaacttcttc

SEQ ID NO: 15 - DNA, 56 bp, synthetic construct
  source 1..56: mol_type = other DNA
gaagttaagaaaatccttgcttaaatttaactccttaagttactttaatgatttag

SEQ ID NO: 16 - DNA, 41 bp, synthetic construct
  source 1..41: mol_type = other DNA
tcgaaggctttaatttgcgcgaaaagccaattagtgtgata

SEQ ID NO: 17 - DNA, 46 bp, synthetic construct
  source 1..46: mol_type = other DNA
tagtatcacactaattggcttttcgcgcaaattaaagccttcgagc

SEQ ID NO: 18 - DNA, 49 bp, synthetic construct
  source 1..49: mol_type = other DNA
gggacgcgccctgtagcggctgaggtctcaacaggccccttttcctttg

SEQ ID NO: 19 - DNA, 55 bp, synthetic construct
  source 1..55: mol_type = other DNA
catgatatcgacaaaggaaaaggggcctgttgagacctcagccgctacagggcgc

SEQ ID NO: 20 - DNA, 52 bp, synthetic construct
  source 1..52: mol_type = other DNA
gaatttttgaaaattcaatataaatgtgagaccaccatgattacgccaagcg

SEQ ID NO: 21 - DNA, 57 bp, synthetic construct
  source 1..57: mol_type = other DNA
taatcatggtggtctcacatttatattgaattttcaaaaattcttactttttttttg

SEQ ID NO: 22 - DNA, 62 bp, synthetic construct
  source 1..62: mol_type = other DNA
atctctctctcctaatttctttttctgaagccattatagttttttctccttgacgttaaagt

SEQ ID NO: 23 - DNA, 50 bp, synthetic construct
  source 1..50: mol_type = other DNA
ttaacgtcaaggagaaaaaactataatggcttcagaaaaagaaattagga

SEQ ID NO: 24 - DNA, 59 bp, synthetic construct
  source 1..59: mol_type = other DNA
atgtacaaatatcataaaaaaagagaatctttttaaaaaaaatccttggactagtcacg

SEQ ID NO: 25 - DNA, 59 bp, synthetic construct
  source 1..59: mol_type = other DNA
actagtccaaggattttttttaaaaagattctctttttttatgatatttgtacataaac

SEQ ID NO: 26 - DNA, 59 bp, synthetic construct
  source 1..59: mol_type = other DNA
gcgccattcgccattcaggctgcgcaactgttgtttaaacgacaacgaccaagctcaca

SEQ ID NO: 27 - DNA, 50 bp, synthetic construct
  source 1..50: mol_type = other DNA
gatgtgagcttggtcgttgtcgtttaaacaacagttgcgcagcctgaatg

SEQ ID NO: 28 - DNA, 59 bp, synthetic construct
  source 1..59: mol_type = other DNA
tcaacagtatagaaccgtggatgatgtggtttctacaggatctgacattattattgttg

SEQ ID NO: 29 - DNA, 59 bp, synthetic construct
  source 1..59: mol_type = other DNA
atagtcctcttccaacaataataatgtcagatcctgtagaaaccacatcatccacggtt

SEQ ID NO: 30 - DNA, 59 bp, synthetic construct
  source 1..59: mol_type = other DNA
agggcttaccatctggccccagtgctgcaatgataccgcgcgacccacgctcaccggct

SEQ ID NO: 31 - DNA, 59 bp, synthetic construct
  source 1..59: mol_type = other DNA
tgataaatctggagccggtgagcgtgggtcgcgcggtatcattgcagcactggggccag

SEQ ID NO: 32 - DNA, 59 bp, synthetic construct
  source 1..59: mol_type = other DNA
cgatagcgcccctgtgtgttctcgttatgtttaaactactagagctccagcttttgttc

SEQ ID NO: 33 - DNA, 51 bp, synthetic construct
  source 1..51: mol_type = other DNA
acaaaggaaaaggggcctgtttaaaacatggagtttatatattccttgatg

SEQ ID NO: 34 - DNA, 45 bp, synthetic construct
  source 1..45: mol_type = other DNA
tttttgaaaattcaatataaatgatcatcaacaattgtgttacac

SEQ ID NO: 35 - DNA, 50 bp, synthetic construct
  source 1..50: mol_type = other DNA
acaaaggaaaaggggcctgtttaaaacaaacattgtatatgctcctcaag

SEQ ID NO: 36 - DNA, 38 bp, synthetic construct
  source 1..38: mol_type = other DNA
tttttgaaaattcaatataaatggcaccccctccttcc

SEQ ID NO: 37 - DNA, 55 bp, synthetic construct
  source 1..55: mol_type = other DNA
acaaaggaaaaggggcctgtctatgtaacgtttatcatatttaaaacaaacattg

SEQ ID NO: 38 - DNA, 38 bp, synthetic construct; identical to SEQ ID NO: 36
  source 1..38: mol_type = other DNA

SEQ ID NO: 39 - DNA, 55 bp, synthetic construct; identical to SEQ ID NO: 37
  source 1..55: mol_type = other DNA

SEQ ID NO: 40 - DNA, 38 bp, synthetic construct; identical to SEQ ID NO: 36
  source 1..38: mol_type = other DNA

SEQ ID NO: 41 - DNA, 66 bp, synthetic construct
  source 1..66: mol_type = other DNA
ggaaaaggggcctgtttacttatcgtcgtcatccttgtaatcgaacatggagttaatgtattcttc

SEQ ID NO: 42 - DNA, 43 bp, synthetic construct
  source 1..43: mol_type = other DNA
gaatttttgaaaattcaatataaatgacaatctctgttactag

SEQ ID NO: 43 - DNA, 67 bp, synthetic construct
  source 1..67: mol_type = other DNA
ggaaaaggggcctgtttacttatcgtcgtcatccttgtaatccatatcaatagccttgaagaacaag

SEQ ID NO: 44 - DNA, 45 bp, synthetic construct
  source 1..45: mol_type = other DNA
gaatttttgaaaattcaatataaatgtctatcaacatcttgcatg

SEQ ID NO: 45 - DNA, 59 bp, synthetic construct
  source 1..59: mol_type = other DNA
attaaccctcactaaagggaacaaaagcgtttaaacacgcagataattccaggtatttt

SEQ ID NO: 46 - DNA, 54 bp, synthetic construct
  source 1..54: mol_type = other DNA
aatacgactcactatagggcgaattgggtaccttcgtttcctgcaggtttttgt

SEQ ID NO: 47 - DNA, 46 bp, synthetic construct
  source 1..46: mol_type = other DNA
caaaaacctgcaggaaacgaaggtacccaattcgccctatagtgag

SEQ ID NO: 48 - DNA, 50 bp, synthetic construct
  source 1..50: mol_type = other DNA
gttttgggacgctcgaaggctttaatttgctcacagcttgtctgtaagcg

SEQ ID NO: 49 - DNA, 59 bp, synthetic construct
  source 1..59: mol_type = other DNA
ttgtctgctcccggcatccgcttacagacaagctgtgagcaaattaaagccttcgagcg

SEQ ID NO: 50 - DNA, 59 bp, synthetic construct
  source 1..59: mol_type = other DNA
gtttgaaagatgggtccgtcacctgcattaaatcctaaacaggccccttttcctttgtc

SEQ ID NO: 51 - DNA, 59 bp, synthetic construct
  source 1..59: mol_type = other DNA
taattacatgatatcgacaaaggaaaaggggcctgtttaggatttaatgcaggtgacgg

SEQ ID NO: 52 - DNA, 49 bp, synthetic construct
  source 1..49: mol_type = other DNA
gaatttttgaaaattcaatataaatggttttaaccaataaaacagtcat

SEQ ID NO: 53 - DNA, 58 bp, synthetic construct
  source 1..58: mol_type = other DNA
gttttattggttaaaaccatttatattgaattttcaaaaattcttactttttttttgg

SEQ ID NO: 54 - DNA, 50 bp, synthetic construct
  source 1..50: mol_type = other DNA
taacacaattgttgatgatcattatagttttttctccttgacgttaaagt

SEQ ID NO: 55 - DNA, 45 bp, synthetic construct
  source 1..45: mol_type = other DNA
gtcaaggagaaaaaactataatgatcatcaacaattgtgttacac

SEQ ID NO: 56 - DNA, 51 bp, synthetic construct
  source 1..51: mol_type = other DNA
cgatttcaattcaattcaatttaaaacatggagtttatatattccttgatg

SEQ ID NO: 57 - DNA, 51 bp, synthetic construct
  source 1..51: mol_type = other DNA
tatataaactccatgttttaaattgaattgaattgaaatcgatagatcaat

SEQ ID NO: 58 - DNA, 59 bp, synthetic construct
  source 1..59: mol_type = other DNA
ttgaagctctaatttgtgagtttagtatacatgcatttacaacgaacgcagaattttcg

SEQ ID NO: 59 - DNA, 59 bp, synthetic construct
  source 1..59: mol_type = other DNA
gtttaataactcgaaaattctgcgttcgttgtaaatgcatgtatactaaactcacaaat

SEQ ID NO: 60 - DNA, 50 bp, synthetic construct
  source 1..50: mol_type = other DNA
gacggtcacagcttgtctgtgtttaaaccgtttaagggcaaatgtactct

SEQ ID NO: 61 - DNA, 50 bp, synthetic construct
  source 1..50: mol_type = other DNA
agagtacatttgcccttaaacggtttaaacacagacaagctgtgaccgtc

SEQ ID NO: 62 - DNA, 59 bp, synthetic construct
  source 1..59: mol_type = other DNA
tgcttcaaaatacctggaattatctgcgtgtttaaacgcttttgttccctttagtgagg

SEQ ID NO: 63 - DNA, 48 bp, synthetic construct
  source 1..48: mol_type = other DNA
ctaaagggaacaaaagcgcggccgcggcaatttggtacaaaaatcacg

SEQ ID NO: 64 - DNA, 46 bp, synthetic construct
  source 1..46: mol_type = other DNA
ctatattatatatatagtaatgtcgtttttgcgaaaccctatgctc

SEQ ID NO: 65 - DNA, 46 bp, synthetic construct
  source 1..46: mol_type = other DNA
gagcatagggtttcgcaaaaacgacattactatatatataatatag

SEQ ID NO: 66 - DNA, 41 bp, synthetic construct
  source 1..41: mol_type = other DNA
gctgaatgggcagttcgaatacctgatgcggtattttctcc

SEQ ID NO: 67 - DNA, 41 bp, synthetic construct
  source 1..41: mol_type = other DNA
ggagaaaataccgcatcaggtattcgaactgcccattcagc

SEQ ID NO: 68 - DNA, 53 bp, synthetic construct
  source 1..53: mol_type = other DNA
gtaagaatttttgaaaattcaatataagtctgaagaatgaatgatttgatgat

SEQ ID NO: 69 - DNA, 56 bp, synthetic construct
  source 1..56: mol_type = other DNA
aatcattcattcttcagacttatattgaattttcaaaaattcttactttttttttg

SEQ ID NO: 70 - DNA, 42 bp, synthetic construct
  source 1..42: mol_type = other DNA
aacacaattgttgatgatcattatagttttttctccttgacg

SEQ ID NO: 71 - DNA, 45 bp, synthetic construct; identical to SEQ ID NO: 55
  source 1..45: mol_type = other DNA

SEQ ID NO: 72 - DNA, 51 bp, synthetic construct; identical to SEQ ID NO: 56
  source 1..51: mol_type = other DNA

SEQ ID NO: 73 - DNA, 41 bp, synthetic construct
  source 1..41: mol_type = other DNA
tatataaactccatgttttaaattgaattgaattgaaatcg

SEQ ID NO: 74 - DNA, 41 bp, synthetic construct
  source 1..41: mol_type = other DNA
gctcatcccgaccttccattaacgaacgcagaattttcgag

SEQ ID NO: 75 - DNA, 41 bp, synthetic construct
  source 1..41: mol_type = other DNA
ctcgaaaattctgcgttcgttaatggaaggtcgggatgagc

SEQ ID NO: 76 - DNA, 52 bp, synthetic construct
  source 1..52: mol_type = other DNA
agacggtcacagcttgtctgtgcggccgcgcttctaataaaccgatgaacgc

SEQ ID NO: 77 - DNA, 52 bp, synthetic construct
  source 1..52: mol_type = other DNA
gcgttcatcggtttattagaagcgcggccgcacagacaagctgtgaccgtct

SEQ ID NO: 78 - DNA, 47 bp, synthetic construct
  source 1..47: mol_type = other DNA
ttttgtaccaaattgccgcggccgcgcttttgttccctttagtgagg

SEQ ID NO: 79 - DNA, 59 bp, synthetic construct
  source 1..59: mol_type = other DNA
aaaggtctcagatcttttccactgcactttgcatgttttagagctagaaatagcaagtt

SEQ ID NO: 80 - DNA, 42 bp, synthetic construct
  source 1..42: mol_type = other DNA
aaaggtctcaaaactctagactttttcgatgatgtagtttct

SEQ ID NO: 81 - DNA, 25 bp, synthetic construct
  source 1..25: mol_type = other DNA
atagaagacgaacattgtacgatac

SEQ ID NO: 82 - DNA, 50 bp, synthetic construct
  source 1..50: mol_type = other DNA
acccaaaaccgataacgccttccgataagtcggtattgttgttgaagatg

SEQ ID NO: 83 - DNA, 43 bp, synthetic construct
  source 1..43: mol_type = other DNA
tcaacaacaataccgacttatcggaaggcgttatcggttttgg

SEQ ID NO: 84 - DNA, 20 bp, synthetic construct
  source 1..20: mol_type = other DNA
aatttctcgtggaagtgacg

SEQ ID NO: 85 - DNA, 24 bp, synthetic construct
  source 1..24: mol_type = other DNA
caatggtctaggtagtggcattcg

SEQ ID NO: 86 - DNA, 47 bp, synthetic construct
  source 1..47: mol_type = other DNA
cgactcactatagggcgaattgggtacgacgggagtggaaagaacgg

SEQ ID NO: 87 - DNA, 46 bp, synthetic construct
  source 1..46: mol_type = other DNA
tcccgttctttccactcccgtcgtacccaattcgccctatagtgag

SEQ ID NO: 88 - DNA, 45 bp, synthetic construct
  source 1..45: mol_type = other DNA
gccaagcacagggcaagatgctttcacagcttgtctgtaagcgga

SEQ ID NO: 89 - DNA, 45 bp, synthetic construct
  source 1..45: mol_type = other DNA
gcatccgcttacagacaagctgtgaaagcatcttgccctgtgctt

SEQ ID NO: 90 - DNA, 25 bp, synthetic construct
  source 1..25: mol_type = other DNA
gattccatgctaccttccatggttg

SEQ ID NO: 91 - DNA, 50 bp, synthetic construct
  source 1..50: mol_type = other DNA
aggaggaaggagggggtgccattatagttttttctccttgacgttaaagt

SEQ ID NO: 92 - DNA, 38 bp, synthetic construct
  source 1..38: mol_type = other DNA
gtcaaggagaaaaaactataatggcaccccctccttcc

SEQ ID NO: 93 - DNA, 50 bp, synthetic construct
  source 1..50: mol_type = other DNA
cgatttcaattcaattcaatttaaaacaaacattgtatatgctcctcaag

SEQ ID NO: 94 - DNA, 51 bp, synthetic construct
  source 1..51: mol_type = other DNA
catatacaatgtttgttttaaattgaattgaattgaaatcgatagatcaat

SEQ ID NO: 95 - DNA, 42 bp, synthetic construct
  source 1..42: mol_type = other DNA
ggaggaaggagggggtgccattatagttttttctccttgacg

SEQ ID NO: 96 - DNA, 38 bp, synthetic construct; identical to SEQ ID NO: 92
  source 1..38: mol_type = other DNA

SEQ ID NO: 97 - DNA, 47 bp, synthetic construct
  source 1..47: mol_type = other DNA
cgatttcaattcaattcaatttaaaacaaacattgtatatgctcctc

SEQ ID NO: 98 - DNA, 41 bp, synthetic construct
  source 1..41: mol_type = other DNA
catatacaatgtttgttttaaattgaattgaattgaaatcg

SEQ ID NO: 99 - DNA, 1659 bp, synthetic construct
  source 1..1659: mol_type = other DNA
atggacttttttgattgttccagatcttccattattccattgaaggcttcctcccaaattgttccagctgattctactaacaagtggggtactgtcgaatctaaccacaactctgctccaactaccccattaaacgacagaatttacgttgaacacgctcacatggtcaaggatttcaagagaatcatgaacgtcgctggtgaagacccatctgaaggtttggctatcatcgatgctgtccaaagattggttgtcgaccatcatttccaagaagaaattcactccatcctacaaaagcataaggaattggctacctctaccaccacacacggtgactgtatgaccacctctttgagattcagattgttgagacaagaaggttactacgtctctgctgatgttttcgaaggtctaaaagatgaagaaggtaagttcgaccaaaacttgagtggtgacatcaagggtttgatggccttgtacgaagcttctcaattctccatggaaggtgaaaacatcttggatgaagccagagactactctgcccgtttattgaatgcttgtgttacccaattgggtcacgatcaagctcgtattgtcgaacacactctgactcacccacaccacaagtctctagccagattcatggccaagaacttcttgagagatttccattgcactaacggttggattgacgacttgaagaagttggccaaagctgacatcgacatggctcaatccactttccaaaacgaagttgttcaaatttctcaatggtggaaggaattaggcttagctgaagaattgaaatttgctcgtgaccaaccagtcaaatggtacatctggaccatgacttgtgacgaagatccatctttctccgaattgagaatcaacttgaccaagccaatctctttcgtctacttgattgatgacatcttcgatgtttatggtaccttgcaagaagtcactgatttcaccgaagctgttcacagatgggaccacgacgccattgaacaattgccataccacatgaagatttgtttcaaggctttggacgatatcaccaacgaaatctcctacaaggtctacaagcaatacggatggaaccctttagactccctcagaaagtcttggggtagattgtgtaacgctttcctaactgaagccaagtggttcgcttctggtcatttggctaaggctgaagaatatttagaaaacggtatcatttcctctggtgttcctgttgttttgttgcacttcttttttcttagtggtgaaggtgttactcaaaagtccgttgaaaccattgacaacactccagacattgtctccgctgcagccgctatcttgcgtttatgggatgatttgggttcagctaaggacgaagaccaagatggtaaggacggttcttacttggcctgttacactaatgaaaacccaggttgttctttggaagatgctgagaagcacgttaagtccaagatctgtgatgaatggaaattgttgaacaaggagtgtttgtctcaaaagaacccattctctggttccttccacagagcttgcttgaatgttgctagaatggttccattgatgtacgaatacgacaagaacagaagattgccaagattggaagaattcttgaagagtatcttgatcaaatga

SEQ ID NO: 100 - DNA, 1803 bp, synthetic construct
  source 1..1803: mol_type = other DNA
atgaacttgccaatgactctaagctccaagtccttattcacttcttccagagcctctatcagctcttctttcaagactatttcccaaattgaaattgctaaaactatcgccaactgtgtcccattgattccagaacaccacaagtggtctattcacgatgtttaccacaaattaagattttccaacactacaaccccaccatttaagcaattgaatcatttgaccaagccatgcaccccaaacgaccacatttctaaggaacacgaaagaaaggtcaaggctttcaagaggattttcaacattgttggtgaagacccaatcgaatccttggtcatcattgacgctgtccaacgtctcggtattgaccatcacttccaagaagaaattaacaccattcttcaagaattgtactacactgaagctaacaacgctcacatcggtggtggtggtggttacttctcccacttgcacgaagtttcgctaagattcagattgttgcgtcaacaaggttgttacgtttctccagaagtctttgactgtttcaaggacaacaacggtaagttcaaagaaaacttgggtggtgacatcaacggtttgatggccttatacgaagcctcccatttgtgtttggaaggtgaaagtctattgcaagaagctcgtgaatgttctgcccaattattaaagaccatcgtcactaacttgaatcactctcaagctaccatggtcgaacacactttgtctcatccacaccacaagtctttctctaaattcaccggtaagaagtacttgtctcacggtttccaagataccaacaatggctggttgaacatcttgaagggtttggctaagattgatttcgacatggctcaatccacttaccaaaaggaaattgtccaaatctctaaatggtgggaagaattgggtttggccaaggaaatgtccttcgccagagatcaaccattaaagtggtacatctggtctatggcttgtatcaccgacccatccttatctgaagaaagaattgacttgactaagtccatctcattcatatacttgattgatgatatcttcgatttgtacggtactcaagacgaattaaccttgtttacccaagttgttaacagatgggactacgatgctagtgaacaattgcctcattacatgaagatttgtttcaaggctttggacgatattatcaacgaaatttcttttaagatctacaagaagcacggatggaacccagttgaatctttgagaaagtcttggggcagactatgctctgcattcttggttgaaggtagatggttcgcttcaggttatgtcccagcttccgaagaatacttgaagaacggtatcatctcttctggtgttcatgttgtctgtgttcaattgttcttcttgttgggtagacaagatataattactaagcaatctgtcgaaatcattgacaacgatccagccatcgtctcctctactgctcaaatcttgagattgtgggatgacctgggttctgctaaggatgaggaccaagatggtcacgacggttcctatttagactactatttgaaagagcaccaaggttgttccgttaaggatgctgaagaacacgttatgtctaagatctctgacgcttggaagcaattgaacaaggaatgtatgacgccaaccccattctctcaatgtttcatcaaggctactttgaacttggctagaatggttcctttgatgtactcttacgacaacaaccaaagattaccatctttggaagataacgttaaatctatcttgtccattgctttgtga

SEQ ID NO: 101 - DNA, 1659 bp, synthetic construct
  source 1..1659: mol_type = other DNA
atggctttcttcggttcctctagatcttcaattattccactaaagactattagtcaaattgttactgctgactccaccaacaagagaggtaccgttgattctaaccacaagtccgctccaaccactcctttgaacgaccgtatctgcaccgaacacgctcacaaggtcaaggatttcaagcaaatcatcaacattgctggtgaagatccatctgaaggcttggctatcattgatgctgttcaaagattgggtgttgaccatcacttccaagatgaaattcacactatcttgcaaaagcattacaccctcgctaccggtacgaccacccatggtgactgtatgatcacttctttgagattcagattacttagacaggaaggttactacgtttctgccgatgttttcgaaggtttaaaggatgaagaaggtaagttcgaccaaaacttgtctggtgacatcaagggtttaatggctttgtacgaagcctcccaattgtccatggaaggtgaaaacatcttggacgaagctagagactactcttccagattgttgaacgaatgtgttacccaattggaccacgaccaagctagaatcgtcgaacacactttgacccacccacaccacaaatctctagcccgtttcatggctaagaacttcttaagagacttccacggtactaacggttggattgacgacttgaagaagttggccaaggttgactttgacatggcccaatctacttaccaaaaggaagtcgtccaaatcagtcaatggtggaaggaattgggtttggctgaagaattgaaattcgctagagatcaaccagtcaagtggtacatctggaccacaacttgtcaccaagacccatctttctcggaattgcgtattaacttaaccaagccaatttctttcgtctacttgattgatgatatctttgatgtctacggtaccccacaagaagttactgacttcactgaagctgtcgatagatgggatcatgatgccatcgaccaattgccatactacatgaagatctgtttcaaggctttagatgacataaccaacgaaatctcttacaaggtttacaagcaacacggttggaacccattggattcattgagaaaatcttggggtagattatgtaacgctttcttgactgaagctcaatggttcgcttctggtcacctgccaaaggccgaagaatacttggaaaatggtatcatcagctccggtgtcccagttgttttgctgcatttgttctttttgttgggtgaaggtgtcactcaaaaatccgttgaaatgattgacaacactccagctattgtctctgctgccgctgctattttgagattatgggacgatttgggctctgctaaggacgaagaccaagatggaaaggatggttcttatttggcatgttacactaatgaaaacccaggttgttccttggaagacgctgaaaagcacgttaagtctaagatttgtgatgaatggaaacaattaaataaagaatgtttgtcccaaaagaacccattctctccatcctttttgaaggcttgcttgaacgtttccagaatggttcctttgatgtacgaatatgacgaaaacagacgtttgccaagattggaagaattcttgaaatccatcttaatcaagtaa

SEQ ID NO: 102 - DNA, 1722 bp, synthetic construct
  source 1..1722: mol_type = other DNA
atggccactgccgctggtccaattgccaccaacaactccccacaaaactctaacgcttacagaaccccaatcgctccatctgttccaatcacccacaagtggtctattgctgaagacttgacttgtatctccaacccatccaagcataacaaccctcaaaccggttacagatctttttctgatgaattatacgttaagtacgaagaaaagttggaagatgtcagaaaggctttgagagaagttgaagaaaacccattggaaggtttagttatgattgacgctttgcaaagattgggaattgattatcacttcagaggtgaaatcggtgctttcttgcagaagcaacaaatcatttcttctactccagatggttacccagaacacggtttatatgaagtctccactttattcagattcttgagacaagaaggtcacaacgttactgctgatgttttcaacaacttcaaggacaaggaaggtcgtttccgttcagaattgtctaccgacatcagaggtttgatgtctttgtacgaagcttctcaattaagaattgaaggtgaagacatcttggaccaagctgctgacttctcctcccaattgttgggtagatggaccaaggatccaaaccatcacgaagccagattggtttctaacactttgactcacccataccacaagtctttggccactttcatgggtcaaaaattgtcctatatgaactgtaagggtccaaactgggacggcgttgacaatttgcaagaactcgctaagatggatttgaccattgttcaatctattcaccaaaaggaagtcttccaagttagtcaatggtggaaagacactggtttggctaatgaattgaagctagccagaaaccaaccattgaagtggtacatgtggccaatggctgccttgaccgacccaagattttctgaagaaagagttgaattgactaagccaatctcttttatctacattattgatgatatcttcgacgtctacggtactattgaagagttgactttgttcaccgatgccgtcaacagatgggaattgtctgctgttgaacaattgccagactacatgaaggtttgtttcaaagctttgtacgacgtcactaatgaaattgcttacaagatatacaagaagcacggtcaaaacccgattgactccttgcaaaagacctgggcttcattgtgtaacgcatttctggtcgaagctaaatggttcgcttccggtcatttgccaaatgctgaagaatacttgaagaacggtatcatctcgtctggtgtccacgttgttttggctcacatgttcttcttattaggtgacggtatcactcaagaatccgtcgatttggtcgacgactacccaggtatctccacctccatcgctaccatcttgagattatccgacgatctaggttctgccaaggacgaagaccaagatggttacgatggttcctacatcgaatgttacatgaaagaacataagggttcttctgttgactccgctagggaagaagttatcagaatgatctctgaagcttggaagtgtttgaacaaggaatgcttgtctccaaacccattctctgaatctttccgtattggttcgttaaacatggctagaatgattccaatgatgtactcttacgatgacaaccacaaccttcctatcttggaagaacacatgaaggctatgatatacaacacctctctataa

SEQ ID NO: 103 - DNA, 1560 bp, synthetic construct
  source 1..1560: mol_type = other DNA
atgaacgtcgaaactaagcacactagaaccatgggtgacatttttgtccaacactctcaaaagttggaactgttgaagacagtcttgagaaatgttgctgaattggacgctcttgaaggtttgaacatgattgatgctgtccaaagattaggtattgactacaacttccaaagagaaattgacgaaatcttacacaagcaaatgtccatagtttctgccagagatgacttgcacgaagtcgctttgagattcagattgttgagacaacacggttacttcgttccagaagatgttttcaacaacttcaaggactccaagggtactttcaagcaagttttgggtgaagacatcaagggcttgatgtctttatacgaagcttctcaattgggaactgaaggtgaagacattttggtggaagctgaaaagttctctggtcacttgttaaagacttctttgtctcacttggatcatcacagagtcagaattgttgctaacactttgcggaacccacatcacaagtctttggctccattcatggctcgtaactttttcgtcacctcccaagctaccaactcttggctaaacttgttgaaggaagttgccaagactgatttcaacatggttagatctctccaccaaaatgaaattgtccaaatgtccaagtggtggaaagaattgggtttagcaaaggaattaaaatttgctagagatcaaccattaaagtggtacatctggtctatggcctgtttgactgacccaaagttgtccgaagaaagagttgaattgaccaagccaatcagtttcgtctacttgattgacgatatcttcgatgtctacggtaccttggatgacttgatcttgttcaccgaagctgttaacagatgggaaatcactgctatcgaccatctgccagactatatgaagatctgtttcaaggctttgtacgacatgactaacgaattctcctccaaggtttacttgaagcatggttggaacccattgcaatctttgaagatttcctgggctagcttgtgcaatgctttcttggttgaggccaaatggttcgcttctggtaagctcccaaagtctgaagaatatcttaagaacggtatcgtctcttccggtgtcaacgttgttttagttcacatgttctttttactaggtcaaaacatcaccagaaagtccgttgaattgttgaacgaaaccccagctattatttcttcttcagctgctattttgcgtttgtgggatgatttgggttctgctaaggatgaaaaccaagatggtaatgacggttcctacgttagatgttacttggaagaacacgaaggttgttctattgaagaagccagagaaaagaccatcaacatgatctctgacgaatggaagaagttgaaccgtgaattattgtctccaaaccctttcccagcctccttcactttggcttcattgaacttggctagaatgatccctttaatgtactcttacgatggtaaccaatgtttgccatccctaaaagaatacatgaaattgatgttgtacgaaaccgtctccatgtaa

SEQ ID NO: 104 - DNA, 1641 bp, synthetic construct
  source 1..1641: mol_type = other DNA
atggccaccaccactacaactatctcaaccacacacaaatggtctatctctgttgctgaagacttgaagtgtttgactcctcattgtactttcaccaacgaaatcttagctaagcaagaagaacaattggaagaagtcagatctgtcttgagaaaggttagagaagaagaagactctctaaagcgtttggttatgatcgacgctttgcaacgtctgggtattgactaccacttcagagaagacatcgaagctgttttgcactcccaatacatgaagtcctcttcccactctttcacttaccaacacgaagatttgtacgaagtttccactagattcagattgttgcgtcaacaaggccacaacgttccaactgatgttttcgataagttcaaggacaaggatggtagatttaagaccgaattgtctgaagacatcagaggtttgatgggtttatacgaagcctcccagttatctattgaaggtgaagatattttggaccaagctgccgacttctctgctcaacaattgaacggttggttgaccagattgggtaaccatcaccaagccaaggttatctctaacactcttggtcatccacaacataaatcattggctaaattcatggctaagaattttttgtctgactacaacggtccaaacgccaatgttttgcaagaactagcgaagatggacttcaacatgtctccatccatgtaccaaaaggaaatgttgcaagtctctaagtggtggaaatccactggtttgggtaacgaattaaagtacgctagaaaccaaccattgaaatggtacttgtggtccatggctactttaacccacccatctttgtctgaacaaagagttgaattgaccaagccaatctctttggtctacattattgacgacatcttcgacgtttatggtaccttggaacaattaattttgttcactgaagctgttaacagatgggaattggccgctgtcaatgaattgccaggttacatgaagatctgtttcaaggtcttgcaagatagtactaatgaaattgcttacggtgtccacaagaagtacggttggaacccaattgatagtttgagaagaacctgggctatcttgtgtaacgctttcttggaagaagctaagtggttcgcttctggtcaattgccaaaggctgaagaatacttaaagaacggtatcgtctccagcggtgtcccagctgtcctcgtccacatgtttttcttgttgggtcacggtatgaacaaggaatctgtcgattttaccatcgataacccagacattatcacttctaccgccaccattttgagattatgggacgatctgggttctgcaaaggatgaaaaccaagacggtcacgatggttcttacgttgattgctatatgaaagaaaacgaaggttccaccgctgaaactgctagaaagcaagttgttgacatgatctctgatgcttggaagagattgaaccgtgaatgtttgtctccaaacagattctccgctaacttcactcaaggttgtctcaacactgccagaatggttccattgatgtactgttacgatgacaaccacaacttgcctattttggaggagcacatgaagtccatgttccacgactccgtcagcttatga

SEQ ID NO: 105 - DNA, 1482 bp, synthetic construct
  source 1..1482: mol_type = other DNA
atgaagaaggaattatccacacgtcactctgaagccttggaagaagtcagaaacctattgaacaatggtccatttaagaacttgcacatggttgacactttgcaaaagttgtgtatcgaccaccacttccaagaagaaattgacatgattttcaagatgtccaacgttcatgactaccaaaccctatacgccttatccttatatttcagaatctttagacaacaaggttactacgttccagctgacgttttcgctaactttaagcaaaaggatggtagattcaaaggtgaaattgctcaagatttgaagggtttattagctttatacgaagcttctcaattatctattccaggtgaagacattttggaggaagctgctgatttctcaagatacttgttggaacaaaagatcccatttttgggtcaagaagaagccctcaaggttaacaacaccttggaacactcctaccaaaagatcccatctaacttcagattgaagatcttcatcaagcactactctggtaccaccatgtctgacttggctatgctggaattggctaaggccaagtctgttcacgctactgaagttgaacaattctccagatggtggaaggatctaggtttggctcaagaattgaaattggccagaaaccaaccattgcactggtacttgtggccaatggctggtttgactgacttgtctttgtctgaaatcagaattgacttgaccaagccaatcgctttggtcttcattattgatgacatcttcgatgtctacggtactttggaccaattgattttattcactgaagctgttaccagatgggattccaataacttggaacaattgccatacaatttgagaacttgtgttcaagctgtctttgatgttactcacgaaatctctgataaaatttacaacaagtacggtttcaaccctattgaattcttgaagcaaacttggactgatttgtgtgaagctttcttagtcgaagcaaagtggttcaccaaaggttacatgccaaaggctgaagattatttaaagaacggtgccgtctccaccggtgctcacgttgtgattgtccacatgttcttcttgttgggtggtgccacctccaaggaaactgctcaaatcattgacgaacacaagggtatcacttcttgcttagccaagatcttgcgattgtgggacgatttgggttctgctaaggacgtcaaccaagacggtcatgacggctcatacgtcacttactatatgaatgaaaactctggatgttccatccaaaacgctcgtgaacatgttatgaagatgatcatcaacacctggaaacaattgaacaaagaatgtttgtctccaaacccattctctgccactttcaccaaggcttgtttgaacttggctagaatgattcctttgatgtacaactacgatgaaaaccatagtttgccattgatcaaggactacattaactccatgttctaa

SEQ ID NO: 106 - DNA, 52 bp, synthetic construct
  source 1..52: mol_type = other DNA
gatatcgacaaaggaaaaggggcctgttcatttgatcaagatactcttcaag

SEQ ID NO: 107 - DNA, 46 bp, synthetic construct
  source 1..46: mol_type = other DNA
gaatttttgaaaattcaatataaatggacttttttgattgttccag

SEQ ID NO: 108 - DNA, 46 bp, synthetic construct
  source 1..46: mol_type = other DNA
gacaaaggaaaaggggcctgttcacaaagcaatggacaagatagat

SEQ ID NO: 109 - DNA, 48 bp, synthetic construct
  source 1..48: mol_type = other DNA
aagaatttttgaaaattcaatataaatgaacttgccaatgactctaag

SEQ ID NO: 110 - DNA, 52 bp, synthetic construct
  source 1..52: mol_type = other DNA
atcgacaaaggaaaaggggcctgtttacttgattaagatggatttcaagaat

SEQ ID NO: 111 - DNA, 44 bp, synthetic construct
  source 1..44: mol_type = other DNA
atttttgaaaattcaatataaatggctttcttcggttcctctag

SEQ ID NO: 112 - DNA, 47 bp, synthetic construct
  source 1..47: mol_type = other DNA
caaaggaaaaggggcctgtttatagagaggtgttgtatatcatagcc

SEQ ID NO: 113 - DNA, 42 bp, synthetic construct
  source 1..42: mol_type = other DNA
gaatttttgaaaattcaatataaatggccactgccgctggtc

SEQ ID NO: 114 - DNA, 49 bp, synthetic construct
  source 1..49: mol_type = other DNA
atcgacaaaggaaaaggggcctgtttacatggagacggtttcgtacaac

SEQ ID NO: 115 - DNA, 52 bp, synthetic construct
  source 1..52: mol_type = other DNA
taagaatttttgaaaattcaatataaatgaacgtcgaaactaagcacactag

SEQ ID NO: 116 - DNA, 46 bp, synthetic construct
  source 1..46: mol_type = other DNA
gacaaaggaaaaggggcctgttcataagctgacggagtcgtggaac

SEQ ID NO: 117 - DNA, 50 bp, synthetic construct
  source 1..50: mol_type = other DNA
aaagtaagaatttttgaaaattcaatataaatggccaccaccactacaac

SEQ ID NO: 118 - DNA, 48 bp, synthetic construct
  source 1..48: mol_type = other DNA
cgacaaaggaaaaggggcctgtttagaacatggagttaatgtagtcct

SEQ ID NO: 119 - DNA, 52 bp, synthetic construct
  source 1..52: mol_type = other DNA
gtaagaatttttgaaaattcaatataaatgaagaaggaattatccacacgtc

(19) United States
(12) Patent Application Publication
(10) Pub. No.: US 2025/0290103 A1
(43) Pub. Date: Sep. 18, 2025

(54) NEROLIDOL SYNTHASE AND USE THEREOF

(71) Applicant: WUHAN HESHENG TECHNOLOGY CO., LTD., Wuhan (CN)

(72) Inventor: Ziling YE, Wuhan (CN)

(73) Assignee: WUHAN HESHENG TECHNOLOGY CO., LTD., Wuhan (CN)

(21) Appl. No.: 18/860,715

(22) PCT Filed: Apr. 26, 2023
(86) PCT No.: PCT/CN2023/090827

§ 371 Date: Dec. 16, 2024

(30) Foreign Application Priority Data
Apr. 29, 2022 (CN) 202210473488.4

(51) Int. Cl. C12P 7/04; C12N 9/88

(52) U.S. Cl. CPC C12P 7/04; C12N 9/88; C12Y 402/03048

(57) ABSTRACT

Provided are a nerolidol synthetase comprising structural domains having Pfam numbers PF01397 and PF03936, and a use thereof. The amino acid sequence of the nerolidol synthase is as shown in SEQ ID NO: 1, SEQ ID NO: 2, SEQ ID NO: 3, SEQ ID NO: 4, SEQ ID NO: 11, or SEQ ID NO: 12, and a nucleotide sequence encoding a nucleic acid molecule is as shown in SEQ ID NO: 5, SEQ ID NO: 6, SEQ ID NO: 7, SEQ ID NO: 8, SEQ ID NO: 9, or SEQ ID NO: 10. The use includes: integrating a coding gene of the nerolidol synthetase into a nucleic acid construct and introducing same into a host cell to obtain a recombinant bacterium, so that the coding gene is expressed in the recombinant bacterium. Thus, biosynthesis of nerolidol is implemented, and the yield of nerolidol is remarkably improved.

### CROSS REFERENCE TO THE RELATED APPLICATIONS

This application is the national phase entry of International Application No. PCT/CN2023/090827, filed on Apr. 26, 2023, which is based upon and claims priority to Chinese Patent Application No. 202210473488.4, filed on Apr. 29, 2022, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBZD037_Sequence_Listing.xml, created on Dec. 16, 2024, and is 133,086 bytes in size.

### TECHNICAL FIELD

The invention belongs to the field of nerolidol biosynthesis, and particularly relates to nerolidol synthase and use thereof.

### BACKGROUND

Nerolidol is a sesquiterpene compound originally found in orange blossom, thus was named nerolidol, and was later found to exist in aromatic plants such as citronella, lavender, lemongrass and ginger. Nerolidol has a wide range of biological functions, such as acaricidal, anti- herbivorous, antibacterial, antioxidant, anti-inflammatory and anti-anxiety activities, and is a promising phytochemical drug. It is also a synthetic intermediate of herbivore-induced volatile substance DMNT, that can protect plants from damage of herbivores. In addition, nerolidol was approved by the U.S. Food and Drug Administration (FDA) as a food flavoring agent.

The synthesis of nerolidol includes three main methods: plant extraction, chemical synthesis and biosynthesis. Plant extraction and chemical synthesis are subjected to various limitations such as complex process, low yield due to seasonal effects, high extraction cost; in addition, plant extraction and chemical synthesis often involve toxic reagents in reactions, which have a great impact on the environment, and possibly higher amount of solvent residues and toxic reagent residues in the products. Thus, the safety of the product needs to be improved. Metabolic engineering and synthetic biology provide an alternative pathway for engineering microbial cell factories to produce nerolidol sustainably and efficiently.

### SUMMARY

The aim of the present invention is to provide a nerolidol synthase and use thereof in the biosynthesis of nerolidol.

In order to solve the technical problems mentioned above, the present application proposes the following technical solutions:

The first aspect of the present application provides a nerolidol synthase including the domains of Pfam Nos. PF01397 and PF03936, and having the activity of nerolidol synthase.

The second aspect of the present application provides a polynucleotide molecule including at least one of the nucleotide sequences encoding the nerolidol synthase in the first aspect of the present application or the complementary sequence thereof.

The third aspect of the present application provides a nucleic acid construct including at least one of the polynucleotide molecules provided in the second aspect of the present application.

The fourth aspect of the present application provides a recombinant strain including the polynucleotide molecule provided in the second aspect of the present application, or the nucleic acid construct provided in the third aspect of the present application.

The fifth aspect of the present application provides the use of nerolidol synthase in the first aspect of the present application, the polynucleotide molecule in the second aspect of the present application, the nucleic acid construct in the third aspect of the present application or the recombinant strain in the fourth aspect of the present application in the production of nerolidol.

The sixth aspect of the present application provides a method for preparing nerolidol, including biosynthesizing nerolidol using the recombinant strain in the fourth aspect of the present application.

The present application identifies a novel nerolidol synthase and the encoding gene thereof, and the encoding gene of the nerolidol synthase is integrated into a nucleic acid construct and introduced into a host cell to obtain a recombinant strain, so that the encoding gene is expressed in the recombinant strain, and in turn the biosynthesis of nerolidol is realized. By adopting the recombinant strain and the method for biosynthesizing nerolidol of the present application, the yield of nerolidol is remarkably improved.

### BRIEF DESCRIPTION OF THE DRAWINGS

FIG. 1 shows a schematic diagram of the construction of plasmid pZY900;

FIGS. 2A-2M show schematic diagrams of the construction of plasmids pYR013, pYR007, pArar-TPS27, pArar-TPS28, pYR006, pYR010, pCaNES2, pCaNES1, pTwNES, pAcNES1, pFaNES1, pCsNES2 and pLpNES1;

FIG. 3 is the extracted ion flow chromatogram of nerolidol characteristic ion m/z=93 in the fermentation products of strains CCJ-1 and S900;

FIG. 4 is the mass spectrum of nerolidol in the fermentation products of strains CCJ-1 and S900;

FIG. 5 is the extracted ion flow chromatogram of nerolidol characteristic ion m/z=93 in the fermentation products of strains LXF-1, LXF-1-1, LXF-1-2 and S900;

FIG. 6 is the mass spectrum of nerolidol in the fermentation products of strains LXF-1, LXF-1-1, LXF-1-2 and S900;

FIG. 7 is the extracted ion flow chromatogram of nerolidol characteristic ion m/z=93 in the fermentation products of strains AH-1, AH-2 and S900;

FIG. 8 is the mass spectrum of nerolidol in the fermentation products of strains AH-1, AH-2 and S900;

FIGS. 9A-9D show schematic diagrams of the construction of plasmids pYR020, pYR021, pYR017 and pYR018;

FIG. 10A shows a schematic diagram of the construction of plasmid pYH395;

FIG. 10B shows a schematic diagram of the knockout element construction of the upstream activation cis element (−220 to −175) of the ERG9 promoter;

FIG. 11 is the schematic diagram of the construction of pZY521 knockout cassette;

FIG. 12 shows the shake flask fermentation yields of strains including nerolidol synthase CCJ_TPS23;

FIG. 13 shows the shake flask fermentation yields of strains including nerolidol synthase ACH_TPS07.

### DETAILED DESCRIPTION OF THE EMBODIMENTS

The terms and descriptions used herein are only for the purpose of describing particular embodiments and are not intended to limit the application. Unless otherwise defined, all technical and scientific terms used herein have the same meaning as commonly understood by one of ordinary skill in the art to which this disclosure pertains. In addition, unless the context requires otherwise, singular terms shall include the plural and plural terms shall include the singular.

### Definition

As used herein, the terms “a” and “an” and “the” and similar references indicate both singular and plural unless otherwise indicated herein or clearly contradicted with the context.

As used herein, the terms “about” and “similar to” mean within an acceptable error range for a particular value as determined by one of ordinary skill in the art, which may depend in part on how the value is measured or determined, or on the limitations of the measurement system.

The term “nucleic acid” or “polynucleotide” refers to deoxyribonucleic acid (DNA) or ribonucleic acid (RNA) and polymers thereof in single or double stranded form. Unless explicitly limited, the term “nucleic acid” or “polynucleotide” also includes nucleic acids including known analogs of natural nucleotides that have binding properties similar to those of the reference nucleic acid and are metabolized in a manner similar to naturally occurring nucleotides (see, Kariko et. al. U.S. Pat. No. 8,278,036, which discloses mRNA molecules in which uridine is replaced by pseudo uridine, methods of synthesizing the mRNA molecules, and methods for delivering therapeutic proteins in vivo). Unless otherwise indicated, a particular nucleic acid sequence also implicitly includes conservatively modified variants thereof (e.g., degenerate codon substitutions), alleles, orthologs, single nucleotide polymorphisms (SNPs), and complementary sequences, as well as explicitly indicated sequences.

“Construct” refers to any recombinant polynucleotide molecule (e.g., plasmid, cosmid, virus, autonomously replicating polynucleotide molecule, phage, linear or circular single or double stranded DNA or RNA polynucleotide molecule), which may be derived from any source, capable of integrating with the genome or autonomously replicating, which may be operably linked to one or more polynucleotide molecules. In the present application, constructs generally include a polynucleotide molecule of the present application operably linked to transcription initiation regulatory sequences that direct the transcription of the polynucleotide molecule of the present application in a host cell. Heterogeneous promoters or endogenous promoters may be used to direct expression of the nucleic acids of the present application.

“Vector” refers to any recombinant nucleic acid construct that can be used for transformation purposes (i.e., introduction of heterologous DNA into a host cell). The vector may include a resistance gene for growth in an organism and a promoter for expression of the protein of interest in the organism. Certain vectors are capable of autonomous replication in the host cell into which they are introduced (e.g., vectors having an origin of replication that functions in the host cell). Other vectors may be introduced into the host cell and integrated into the genome of the host cell and thus replicated together with the host genome. Furthermore, certain preferred vectors are capable of directing the expression of foreign genes to which they are linked. One type of vector is a “plasmid”, which generally refers to a circular double-stranded DNA loop into which additional DNA segments (exogenous genes) may be ligated, and may also include linear double-stranded molecules, such as those obtained from amplification by polymerase chain reaction (PCR) or treatment of circular plasmids with restriction enzymes.

Plasmid vectors include vector backbones (i.e. empty vectors) and expression frameworks.

The term “expression framework” refers to sequences having the potential to encode proteins.

The term “host cell” refers to a cell capable of introducing a gene of interest and providing conditions for cloning and/or expression of the gene of interest, such as a microorganism, and specifically may be a bacterium (such as Escherichia coli), a yeast (such as Saccharomyces cerevisiae), an actinomycete, or the like.

The term “recombinant strain” refers to genetically engineered strains (such as bacteria, yeasts, actinomycetes, etc.), which means that exogenous gene fragments have been introduced into the strain, wherein one way of modification includes the introduction of new DNA fragments into the genome of the strain to cause a change, and the other way includes the introduction of artificially constructed or modified plasmids into the strain so that the strain acquires the ability to express the target gene.

The first aspect of the present application provides a nerolidol synthase including the domains of Pfam Nos. PF01397 and PF03936, and having the activity of nerolidol synthase.

In some embodiments, the nerolidol synthase is from Tanacetum cinerariifolium, Astilbe chinensis, or Artemisia argyi.

In some embodiments, the nerolidol synthase has the amino acid sequence having at least 90%, 91%, 92%, 93%, 94%, 95%, 96%, 97%, 98%, 99% or 100% sequence identity to the amino acid sequence as shown in SEQ ID NO: 1, SEQ ID NO: 2, SEQ ID NO: 3, SEQ ID NO: 4, SEQ ID NO: 11 or SEQ ID NO: 12.

In some embodiments, the nerolidol synthase has the following amino acid sequences (amino terminal to carboxy terminal):

CCJ_TPS23 (Tanacetum cinerariifolium source)
(SEQ ID NO: 1)
MIINNCVTPKDTQPAPLPSETDIVSNDINIKNELSLKHLELLEEVR
YLLKNYSSEPLDMVDALQKLCINHYYEDEIGLILKLLYARMSNNGD
YKHDKSLYEVSLSFRILRQEGYYVSADVFADFKQKDGKFKEEIAQD
VKGLMALYEASQLSLDGERILEEASDFSSGALKDMVPSLDLHQAMI
ATNTLQHTYQRTSSTFMVKKFMKVYTGTPMCELAQLELTTKVQSLH
RTELEQISRWWKDLGLAQELKLARNQPLHWYLWPMASLTDLSLSEQ
RIELTKPIAFIFLIDDIFDVYGTLDQLVILTQAVNRWESNSLERLP
HHLRICIQALFDVTNEISDNIYKKHGFNPIDFLKQSWINLCEAFLV
EAKWFAEGYMPTAEDYLNNGMVSTGAHVVIVHMFFLLGGGPNIKSA
SVVNENQGITSCLAKILRLWDDLGSAEDVDQDGNDGSYVTYYMKEN
AGCSIQKAHEHVMEMISNTWKQLNAECLYSSHLSRTFTKACLNLAR
MIPMMYDYDENHSLPFIKEYINSMF
ACH-TPS07 (Astilbe chinensis source)
(SEQ ID NO: 2)
MAPPPSSYPPSARNNFTQVGTGLSDTMNEPSTQKWSITSHDGTLVS
SPIILHNSKANNACYTDEFHVDHESKLNEVRDLLNKVGEDYQLEGL
VMIDAIQCLNIDYRFRKEIESILQSQDETSRVHENDEHDLYQTALR
FRLLRQEGHFVSAEVFDKFKNKEGKFKQELVEDIRGMMGLYEAAQL
RIEGEHILDEAESFCRRLFKGCMPYLNSHEAKLVESTLKHPYRKSL
SRIGAKNFANNFQGNFKWITVLEELSNLDLKIVQSIHQKELQQISH
WWEELGLAKELKLARDQPLKWHMWSMEVLQDPSLSEQRIELTKPIS
LVYIIDDIFDVYGTLDELTLFTEAVTRWEFTDANQLPNYMRTCFQT
LFDITYEIGYKFFIVHGWNPIDTLRKSWVTLCKAFLAEAKWFASGE
LPKADKYLKNGIISSGVHVVLVHMFFLLGQDLTKECLDLVNDNNNI
PGIINYTATILRLWDDLGSAKDENQDGYDGSYLECFMNEHKGLLMK
TAREHMIGMISDAWKCLNKECLSPNPFSSSFTKASLNAARMVPLMY
NYDDNHCLPDLEEHIQCLF
Arar-TPS27 (Artemisia argyi source)
(SEQ ID NO: 3)
MTISVTRKDTQPTPFPSETDIVSNDINIKNELSIRHLELLEEVRNL
LKNCSSKPLVMVDTLQKLCINHHYEEEIGLILKSLYTRMSNNDDYK
HDKSLYEVSLSFRILRQHGYYVSADVFANFKQKDGKFKGEITQDLK
GLIALYEASQLSLEGEQILKEASDFSSGALKEMMPSLDQDQAMIVT
NTLQHTYQRTTSTFMVKKFMKVYTGTPMCELAQLELTKVQSLHRTE
VDQISRWWKGLGLAQELKLARNQPLHWYLWPMASLTDLSLSEQRVE
LTKPIAFIFLIDDIFDVYGTLDQLVILTQAVNRWESNRLEQLPYHL
RICIQALFDVTNEISDKIYKKHGFNPIEFLKQSWINLCDAFLVEAK
WFAEGYMPTAEDYLNNGMVSTGVHVVIVHMFFLLGGGPNIKSASVV
NENQGIMSCLAKILRLWDDLGSAKDVDQDGNDGSYVTYYMKENAGC
SIQKAHEHVMEMISNTWKQLNAECLYSSHFSRTFTKACLNLAKMIP
MMYDYDENHSLPFIEEYINSMF
Arar-TPS28 (Artemisia argyi source)
(SEQ ID NO: 4)
MSINILHGDLPDVKVMSPQADATEKIDELKEKIRRVLMTTSDPKMS
LKLVDTIQRLRIGYYFQEDINEILEKLKQCLPDDELHIVALCFRLL
RQNGIPTNSEVFRKFIDMNGEFIKSTSEDIEGLLSLYEASYMGSNE
EIFLVHAKKITTRELNICVPKLSPKLSKKVLQALELPMHLRMETLE
ARRYIEDYGNEEDHNPLLLELAKLDYNHVQSLFRRELVEMARWWNH
LGIARKFSFVRDRHVECFLWTVGVLPEPKYSATRIVMAKITSILLL
LDDIYDTYGSYDDLVLLTKIIQRWDMTEIEQLPEYMQACYMALYNT
TSEICDKVLRERGLYVEQFLRKTWIKIVEGYMVEVKWLKTGTIPNF
KEYMDNAVTTSGSYMAFVHMFFLICDEVNKENMADLLEPYPKFFTL
AGTILRLWDDLGTVKEEQERGEVLSSIQLLMKEKKITCDKDERKQI
LELIHELWKDLNAELVAPNAVLWPMIRVALNMSRTSQVVYQHNEDS
YLSSVKDHVKNLFFKAIDM
ACH-TPS08 (Astilbe chinensis source)
(SEQ ID NO: 11)
MAPPPSSYPPSARNNFTQVGTGLGDTMNEPSTQKWSITSHDGTLVS
SPIILHNSKANNACYTDEFHVDHERKLNEVRDLLNKVGEDYQLEGL
VMIDAIQCLNIDYHFRKEIESILQCQDETSRVHENDEHDLYQTALR
FRLLRQEGHFVSAEVFDKFKNKEGKFKQELVEDIRGMMGLYEAAQL
RIEGEHILDEAESFCRRLFKACMPYLNSHEARLVESMIKHPYRKSL
SRIGAKNFANNFQGNFKWITVLEELSNLDLKIVQSIHQKELQQISH
WWEELGLAKELKLARDQPLKWHMWSMEVLTDPSLSEQRIELTKPIS
LVYIIDDIFDVYGTLDELTLFTEAVTRWEFTDVNQLPNYMRTCFQT
LFDITYEIGYKFFIVHGWNPINTLRKSWVTLCKAFLAEAKWFASGE
LPKADKYLKNGIISSGVHVVLVHMFFLLGQDLTKECLDLVNDNNNI
PGIINYTATILRLWDDLGSAKDENQDGYDGSYLECFMNEHKGLLMK
TAREHMIGMISDAWKCLNKECLSPNPFSSSFTKASLNAARMVPLMY
NYDDNHCLPDLEEHAMFVLNMINVT
ACH-TPS09 (Astilbe chinensis source)
(SEQ ID NO: 12)
MAPPPSSYPPSARNNFTQVGTGLGDTMNEPSTQKWSITSHDGTLVS
SPIILHNSKANNACYTDEFHVDHERKLNEVRDLLNKVGEDYQLEGL
VMIDAIQCLNIDYHFRKEIESILQCQDETSRVHENDEHDLYQTALR
FRLLRQEGHFVSAEVFDKFKNKEGKFKQELVEDIRGMMGLYEAAQL
RIEGEHILDEAESFCRRLFKACMPYLNSHEARLVESMIKHPYRKSL
SRIGAKNFTNNFQGNFKWITVLEELSNLDLKIVQSIHQKELQQISH
WWEELGLAKELKLARDQPLKWHMWSMEVLTDPSLSEQRIELTKPIS
LVYIIDDIFDVYGTLDELTLFTEAVTRWEFTDVNQLPNYMRTCFQT
LFDITYEIGYKFFIVHGWNPIDTLRKSWVTLCKAFLAEAKWFASGE
LPKADKYLKNGIISSGVHVVLVHMFFLLGQDLSKECLDLVNDNNNI
PGIINYTATILRLWDDLGSAKDENQDGYDGSYLECFMNEHKGLLMK
TAREHMIGMISDAWKCLNKECLSPNPFSSSFTKASLNAARMVPLMY
NYDDNHCLPDLEEHAMFVLNMINVT.

The second aspect of the present application provides a polynucleotide molecule including at least one of the nucleotide sequences encoding the nerolidol synthase in the first aspect of the present application or the complementary sequence thereof.

In some embodiments, the polynucleotide molecule includes the nucleotide sequence having at least 90%, 91%, 92%, 93%, 94%, 95%, 96%, 97%, 98%, 99% or 100% sequence identity to the nucleotide sequence as shown in SEQ ID NO: 5, SEQ ID NO: 6, SEQ ID NO: 7, SEQ ID NO: 8, SEQ ID NO: 9 or SEQ ID NO: 10.

In some embodiments, the polynucleotide molecule includes the nucleotide sequences (5′ end to 3′ end) as shown below:

CCJ_TPS23 (Tanacetum cinerariifolium source)
(SEQ ID NO: 5)
atgatcatcaacaattgtgttacaccgaaagacacccagccagcccctc
tcccatctgaaacagatattgtgtcaaatgacattaacattaagaatga
attgtccctcaaacatctagaattgttagaagaagtccgatatcttcta
aagaattattcttccgaacctttggacatggttgacgctcttcaaaaac
tttgcatcaaccattactatgaagacgagattggcttgattttgaaatt
gctctacgctagaatgtcgaataatggcgactacaagcatgataaaagt
ctttatgaggtttccctttcttttcgcatccttcgtcaagaaggttact
atgtctcagctgatgtttttgctgatttcaaacaaaaggatgggaagtt
taaagaagaaatagcacaagatgttaagggtctaatggcgttatatgaa
gcttcacagctaagcctggatggagaacgaatacttgaagaggcttcag
attttagcagtggtgctcttaaagacatggtgccttctttagatctaca
tcaagctatgattgctactaacacattacaacatacgtatcaaagaacc
tcatcaactttcatggttaaaaaattcatgaaagtatacacgggtacac
ccatgtgtgaactagcccaattagagttgactactaaagtccagtcact
acatcgaacagaactagaacaaatctccagatggtggaaggacttgggg
ttggcgcaagagttgaagcttgcaagaaaccaaccgttgcactggtact
tatggccaatggctagcctcacagatctcagcttgtccgagcagagaat
tgagctcacgaagcccattgctttcatctttctaattgatgacattttt
gatgtttatggaactctagaccaacttgttatcttgacacaagcagtga
atagatgggaaagtaacagccttgaacgactcccacatcacctaaggat
ctgtattcaagctctatttgacgtaacaaacgaaataagtgacaatatc
tacaagaagcatgggttcaatcccattgattttttgaaacaatcgtgga
taaacctttgtgaagcttttctagtagaggcgaaatggtttgctgaggg
atacatgccgacggcagaggattacttgaataatgggatggttagtaca
ggggcacatgtcgtgatagtgcacatgttcttcctcctcggtggtggcc
ctaacataaaaagtgctagcgttgtaaacgaaaaccaagggattacgtc
ttgtttggcaaagattcttcgtctttgggatgacttaggaagtgctgag
gatgtggatcaagacggtaatgatggatcatatgtgacatattacatga
aagagaatgcaggttgttctattcaaaaggcacatgaacatgttatgga
aatgatatcgaatacttggaagcaactaaatgcggagtgcctttactca
agtcatttatcacgtacattcaccaaagcttgcctcaatcttgcaagaa
tgattccaatgatgtacgattatgatgagaatcattcccttcctttcat
caaggaatatataaactccatgttttaa
ACH-TPS07 (Astilbe chinensis source)
(SEQ ID NO: 6)
atggcaccccctccttcctcctatcctccaagtgctcgaaacaatttta
cacaagttggcacaggacttagtgatacgatgaacgagccttctactca
gaaatggagcattaccagccatgatggcaccttagtttcaagccctata
atacttcacaactctaaagcaaacaatgcttgttatactgatgaatttc
atgtcgatcatgaaagcaaattgaatgaagttagggatttgcttaacaa
agttggagaagattatcaattagaaggtttggtcatgatcgacgccatt
caatgcctaaacattgactatcgcttccgaaaggagattgagtccatcc
tacaaagtcaggatgagacatctagggttcatgaaaatgatgaacacga
tctttaccagactgcacttcgttttcgactgctgagacaagaaggccat
tttgtgtctgcagaagtgtttgacaaattcaagaacaaagaggggaagt
ttaagcaagaactagttgaagatataaggggaatgatgggcttatatga
agctgcgcagcttagaattgaaggagaacatatacttgatgaagccgaa
agcttttgtcgtcggctctttaaaggatgcatgccctatcttaactctc
atgaagcaaaacttgtcgagagcacgctaaagcatccctatcgaaagag
cttgtcacgcataggggccaaaaacttcgctaacaatttccaaggcaat
tttaaatggattactgttcttgaagaactatcaaatttagatttgaaaa
tagttcaatctatacaccagaaggaactacaacaaatttcccattggtg
ggaagaacttggtttagcaaaggaattgaagctcgcaagagaccaaccg
ctaaaatggcacatgtggtccatggaagtgttacaagatccaagcttgt
ctgagcaaaggattgagcttacaaagcccatttctcttgtatacataat
tgatgacattttcgacgtttatgggacgcttgatgaactcactctcttc
acggaagcagtcactagatgggaattcaccgacgcaaatcaactgccta
actacatgaggacatgtttccagactctctttgatatcacttatgaaat
tggctacaagtttttcatagttcatggttggaaccctatagacacacta
cgaaaatcgtgggttactttgtgcaaagcatttttggcggaagcaaaat
ggtttgcttctggggaattgccaaaggcagataaatacttgaaaaatgg
aattattagttcaggagtgcatgtagtacttgttcacatgttttttctc
ttgggtcaagatttaacaaaggaatgtttagacctagtgaacgacaaca
acaatattccaggcatcataaactatacggcaacgattcttcgtctttg
ggatgacttgggaagtgcaaaggatgagaatcaagatgggtacgacgga
tcatatctggaatgtttcatgaatgaacacaaaggtttattaatgaaga
ctgcaagagagcatatgattggcatgatttcggatgcatggaagtgctt
gaacaaggaatgcctctctccaaatccattttcatcatctttcacaaag
gcttctcttaatgctgcaaggatggttccattgatgtacaattacgacg
acaaccattgtctcccagatcttgaggagcatatacaatgtttgtttta
a
Arar-TPS27 (Artemisia argyi source)
(SEQ ID NO: 7)
atgacaatctctgttactagaaaggatactcaaccaacaccatttccat
ctgaaactgatattgtttctaacgatatcaacatcaagaacgaattgtc
tattagacatttggaattgttggaagaagttagaaatttgttgaagaac
tgttcttctaagccattagttatggttgatactttacaaaagctgtgta
ttaaccatcattacgaagaagaaatcggtttgattttgaaatctttgta
cacaagaatgtccaataacgatgattataagcatgataagtccttatac
gaagtttctttgtcttttagaatcttgagacaacatggttattacgttt
ctgctgatgtttttgctaattttaagcaaaaggacggtaaatttaaggg
tgaaattacacaagatctgaaaggtttaatcgctttatatgaagctagt
caattgtctttggaaggtgaacaaattttaaaggaagctagtgatttct
cttctggtgctttgaaagaaatgatgccatctttggatcaagatcaagc
tatgattgttacaaatactttgcaacatacctaccaaagaacaacttct
acttttatggttaagaagttcatgaaggtttacactggtacaccaatgt
gtgaattggctcaattggaattgactaaagttcaatctttgcatagaac
agaagttgatcaaatttctagatggtggaaaggtttaggtttggctcaa
gaattaaaattagctagaaaccaaccattgcattggtatttatggccaa
tggcttctttgactgatttgtctttgtctgaacaaagagttgaattgac
taagccaattgcttttattttcttgatcgatgatatcttcgacgtttat
ggtactttggatcaattagttatcttgactcaagctgttaatagatggg
aatctaatagattagagcaattaccataccatttgagaatttgtatcca
agctctatttgatgttacaaatgaaatctctgacaagatttacaagaag
catggttttaatccaatcgaatttttgaagcaatcctggattaatttgt
gtgatgcttttttggttgaggctaaatggtttgctgaaggttatatgcc
aactgctgaagattatttaaacaatggtatggtttccacaggtgttcat
gttgttattgttcatatgtttttcctgttgggtggtggtccaaatatta
aatctgcttctgttgttaacgagaatcaaggtattatgtcttgtttggc
taaaattttgaggttatgggatgatttaggttctgctaaagatgttgat
caagatggtaatgatggttcttatgttacatattacatgaaggaaaacg
ctggttgttctattcaaaaagctcatgaacatgttatggaaatgatttc
taacacttggaaacaattgaacgctgaatgtttatattcctctcatttt
tctaggacattcactaaagcatgtttgaatttggctaaaatgatcccaa
tgatgtatgattatgacgaaaatcattccttgccatttattgaagaata
cattaactccatgttctaa
Arar-TPS28 (Artemisia argyi source)
(SEQ ID NO: 8)
atgtctatcaacatcttgcatggtgatttgccagatgttaaagttatgt
ctccacaagctgatgctacagaaaaaattgatgaattaaaggagaagat
caggagagttttgatgactacatctgatccaaaaatgtctttaaagttg
gttgatactatccaaagattgagaattggttattacttccaagaagata
tcaacgaaatcttggaaaaattgaagcaatgtttgccagatgatgaatt
gcatattgttgctttgtgttttagattgttgagacaaaatggtatccca
acaaattctgaagtttttagaaagttcatcgacatgaatggtgaattta
ttaagtctacttccgaagatatcgaaggtttgttatctttgtatgaagc
tagttatatgggttctaatgaagaaattttcctggttcatgctaaaaaa
atcacaactagagaattgaacatctgtgttccaaaattgtctccaaaat
tgtcaaaaaaggtcttgcaagctctagaattgccaatgcatttgagaat
ggaaacattggaagctagaagatatattgaagattacggtaatgaggaa
gatcataatccattattactggaattggctaaattggattacaatcatg
ttcaatctctgtttagaagagaattagttgaaatggctagatggtggaa
tcatttgggtattgctagaaaattttcctttgttagagacagacatgtt
gaatgttttttgtggactgttggtgttttgccagaaccaaaatattctg
ctactagaattgttatggctaaaattacttccatcttgttattgttgga
cgatatttatgacacttacggttcttatgatgatttggttttattgaca
aagatcatccaaagatgggatatgacagaaattgaacaattgccagaat
atatgcaagcatgttatatggctttgtataatactacatccgaaatttg
tgacaaggttttaagagaaagaggtttgtatgttgaacaatttttgaga
aagacctggattaaaatcgttgaaggttatatggttgaggttaaatggt
taaaaaccggtactattccaaattttaaggaatatatggacaacgctgt
tactacttctggttcttatatggcttttgttcatatgtttttcctgatt
tgtgacgaagttaataaggaaaatatggctgatttgttggaaccatatc
caaaatttttcactttggctggtacaattttgagattgtgggatgattt
aggtactgttaaagaagaacaagaaagaggtgaagttttgtcttctatt
caattattgatgaaggagaagaagatcacttgtgataaagatgaaagaa
agcaaatcttggaattgattcatgaattgtggaaagatttgaacgctga
attagttgctccaaatgctgttttgtggccaatgattagagttgcttta
aatatgtctaggacatctcaagttgtttatcaacataatgaggattctt
acttgtcttctgttaaagatcatgttaagaacttgttcttcaaggctat
tgatatgtaa
ACH-TPS08 (Astilbe chinensis source)
(SEQ ID NO: 9)
atggcaccccctccttcctcctatcctccaagtgctcgaaacaatttta
cacaagttggcacaggacttggtgatacgatgaacgagccttctactca
gaaatggagcattaccagccatgatggcaccttagtttcaagccctata
atacttcacaactctaaagcaaacaatgcttgttatactgatgaatttc
atgtcgatcatgaaagaaaattgaatgaagttagggatttgcttaacaa
agttggagaagattatcaattagaaggtttggtcatgatcgacgccatt
caatgcctaaacattgactatcacttccgaaaggagattgagtccatcc
tacaatgtcaggatgagacatctagggttcatgaaaatgatgaacacga
tctttaccagactgcacttcgttttcgactgctgagacaagaaggccat
tttgtgtctgcagaagtgtttgacaaattcaagaacaaagaggggaagt
ttaagcaagaactagttgaagatataaggggaatgatgggcttatatga
agctgcgcagcttagaattgaaggagaacatatacttgatgaagccgaa
agcttttgtcgtcggctctttaaagcatgcatgccctatcttaactctc
atgaagcaagacttgtcgagagcatgataaagcatccctatcgaaagag
cttgtcacgcataggggccaaaaacttcgctaacaatttccaaggcaat
ttcaaatggattactgttcttgaagaactatcaaatttagatttgaaaa
tagttcaatctatacaccagaaggaactacaacaaatttcccattggtg
ggaagaactgggtttagcaaaggaattgaagctcgcaagagaccaaccg
ctaaaatggcacatgtggtccatggaagtgttaacagatccaagcttgt
ctgagcaaaggattgagcttacaaagcccatttctcttgtatacataat
tgatgacattttcgacgtttatgggacgcttgatgaactcactctcttc
acggaagcagtcacaagatgggaattcaccgacgtaaatcaactgccta
actacatgaggacatgtttccagactctctttgatatcacttatgaaat
tggctataagtttttcatagttcatggttggaaccctataaacacacta
cgaaaatcgtgggttactttgtgcaaagcatttttggcggaagcaaaat
ggtttgcttctggggaattgccaaaggcagataaatacttgaaaaatgg
gattattagttcaggagtgcatgtagtacttgttcacatgttttttctc
ttgggtcaagatttaacaaaggaatgtttagacctagtgaacgacaaca
acaatattccaggcatcataaactatacggcaacgattcttcgtctttg
ggatgacttgggaagtgcaaaggatgagaatcaagacgggtacgacgga
tcatatctggaatgtttcatgaatgaacacaaaggtttattaatgaaga
ctgcaagagagcatatgattggcatgatttcggatgcatggaagtgctt
gaacaaggaatgcctctctccaaatccattttcatcatctttcacaaag
gcttctcttaatgctgcaaggatggttcctttgatgtacaattacgacg
acaaccattgtctcccagatcttgaggaacatgcaatgtttgttttaaa
tatgataaacgttacatag
ACH-TPS09 (Astilbe chinensis source)
(SEQ ID NO: 10)
atggcaccccctccttcctcctatcctccaagtgctcgaaacaatttta
cacaagttggcacaggacttggtgatacgatgaacgagccttctactca
gaaatggagcattaccagccatgatggcaccttagtttcaagccctata
atacttcacaactctaaagcaaacaatgcttgttatactgatgaatttc
atgtcgatcatgaaagaaaattgaatgaagttagggatttgcttaacaa
agttggagaagattatcaattagaaggtttggtcatgatcgacgccatt
caatgcctaaacattgactatcacttccgaaaggagattgagtccatcc
tacaatgtcaggatgagacatctagggttcatgaaaatgatgaacacga
tctttaccagactgcacttcgttttcgactgctgagacaagaaggccat
tttgtgtctgcagaagtgtttgacaaattcaagaacaaagaggggaagt
tcaagcaagaactagttgaagatataaggggaatgatgggcttatatga
agctgcgcagcttagaattgaaggagaacatatacttgatgaagccgaa
agcttttgtcgtcggctctttaaagcatgcatgccctatcttaactctc
atgaagcaagacttgtcgagagcatgataaagcatccctatcgaaagag
cttgtcccgcataggggccaaaaacttcactaacaatttccaaggcaat
ttcaaatggattactgttcttgaagaactatcaaatttagatttgaaaa
tagttcaatctatacaccagaaggaactacaacaaatttcccattggtg
ggaagaactgggtttagcaaaggaattgaagctcgcaagagaccaaccg
ctaaaatggcacatgtggtccatggaagtgttaacagatccaagcttgt
ctgagcaaaggattgagcttacaaagcccatttctcttgtatacataat
tgatgacattttcgacgtttatgggacgcttgatgaactcactctcttc
acggaagcagtcacaagatgggaattcaccgacgtaaatcaactgccta
actacatgaggacatgtttccagactctctttgatatcacttatgaaat
tggctacaagtttttcatagttcatggttggaaccctatagacacacta
cgaaaatcgtgggttactttgtgcaaagcatttttggcggaagcaaaat
ggtttgcttctggggaattgccaaaggcagataaatacttgaaaaatgg
gattattagttcaggagtgcatgtagtacttgttcacatgttttttctc
ttgggtcaagatttatcaaaggaatgtttagacctagtgaacgacaaca
acaatattccaggcatcataaactatacggcaacgattcttcgtctttg
ggatgacttgggaagtgcaaaggatgagaatcaagacgggtacgacgga
tcatatctggaatgtttcatgaatgaacacaaaggtttattaatgaaga
ctgcaagagagcatatgattggcatgatttcggatgcatggaagtgctt
gaacaaggaatgcctctctccaaatccattttcatcatctttcacaaag
gcttctcttaatgctgcaaggatggttcctttgatgtacaattacgacg
acaaccattgtctcccagatcttgaggaacatgcaatgtttgttttaaa
tatgataaacgttacatag.

The third aspect of the present application provides a nucleic acid construct including at least one of the polynucleotide molecules provided in the second aspect of the present application. In the present application, the polynucleotide molecule ligated to the nucleic acid construct is referred to as the target gene, and the enzyme encoded by the polynucleotide molecule is referred to as the target protein.

In some embodiments, the nucleic acid construct further includes regulatory elements for regulating the expression of the target gene, such as promoters, terminators, etc. Exemplary promoters may be constitutive promoters such as P_{TEF1}, P_{TDH3}, P_{GPM1}, P_{TPI1}, etc., inducible promoters such as P_{HXT1} (induced by high concentration of glucose), P_{CUP1} (induced by copper ions), P_{GAL1}, P_{GAL2}, P_{GAL7}, P_{GAL10} (induced by galactose), etc. Those skilled in the art may select as needed, and is not limited herein by the present application.

In some embodiments, the nucleic acid construct further includes a marker gene for screening recombinant strains including a target gene or target protein, such as leucine screening marker, histidine screening marker, tryptophan screening marker, uracil screening marker, etc., which can be specifically selected by a person skilled in the art as needed, and is not limited herein by the present application.

In some embodiments, the nucleotide sequence is located between two insertion elements, wherein the insertion elements are for integrating the nucleotide sequence into the genome of a host cell.

In some embodiments, a nucleotide sequence having insertion elements linked at both ends is ligated to a nucleic acid construct, such as a plasmid backbone of a plasmid vector, and when the nucleic acid construct is used for introducing a target gene into a host cell, the nucleic acid construct can be digested by tools such as a restriction enzyme to obtain a linearized target gene fragment having insertion elements linked at both ends thereof, and the linearized target gene fragment can be introduced into the host cell, so that it can be inserted into the corresponding position of host cell genome through insertion elements at both ends, thus obtaining the recombinant strain of the present application.

A person skilled in the art can introduce the linearized target gene fragment into the host cell by conventional methods, for example, lithium acetate method can be used for yeast, calcium transformation method can be used for Escherichia coli, etc., which are conventional operations in the art, and is not limited herein by the present application.

In some embodiments, the two insertion elements appear in pairs, for example, they can be the left and right homologous arms of Leu2, the left and right homologous arms of Ura3, the left and right homologous arms of YPRCdelta15, etc. The homologous arms of different genes can integrate the gene of interest into different locations in the host cell genome. Those skilled in the art can specifically select the type of homologous arms according to the location they expect to be integrate into in the host cell genome, and is not limited herein by the present application.

In some embodiments, regulatory elements such as promoters and terminators for regulating the expression of the target gene are further included between the two insertion elements. The types of the promoters and terminators are not limited by the present application.

In some embodiments, the nucleic acid construct further includes at least one of the nucleotide sequences encoding acetoacetyl coenzyme A thiolase (ERG10), hydroxy-methylglutaryl coenzyme A synthase (ERG13), hydroxy-methylglutaryl coenzyme A reductase (HMG1), mevalonate kinase (ERG12), mevalonate-5-phosphate kinase (ERG8), mevalonate pyrophosphate decarboxylase (MVD1), isoprene pyrophosphate isomerase (IDI1), farnesyl pyrophosphate synthase (ERG20); wherein the names of the genes encoding these enzymes are shown in parentheses.

In some embodiments, the hydroxy-methylglutaryl coenzyme A reductase is a truncated hydroxy-methylglutaryl coenzyme A reductase (tHMG1), and the endoplasmic reticulum localization sequence is truncated in tHMG1, thus enhance the stability of the enzyme in the cytoplasm.

Exemplary but non-limiting disclosures of the genes encoding the above enzymes are as follows:

ERG10 (Accession/GENE ID: 856079), ERG13 (Accession/GENE ID: 854913), tHMG1 (Accession/GENE ID: 854900, 4-1659bp truncated), ERG12 (Accession/GENE ID: NM_001182715.1), ERG8 (Accession/GENE ID: CP046093.1, 689693 . . . 691048), MVD1 (Accession/GENE ID: NM_001183220.1), IDI1 (Accession/GENE ID: NM_001183931.1), ERG20 (Accession/GENE ID: 853272).

The inventors have found that acetoacetyl coenzyme A thiolase, hydroxy-methylglutaryl coenzyme A synthase, hydroxy-methylglutaryl coenzyme A reductase, mevalonate kinase, mevalonate-5-phosphate kinase, mevalonate pyrophosphate decarboxylase, and isoprene pyrophosphate isomerase belong to enzymes in the mevalonate pathway, and in the mevalonate pathway isoprene pyrophosphate (IPP) and dimethylallyl pyrophosphate (DMAPP) can be synthesized, both of which can be used as precursors for synthesizing farnesyl pyrophosphate (FPP) under the catalysis of farnesyl pyrophosphate synthase, whereas FPP is the substrate in the biosynthesis of nerolidol, thus when at least one of the enzymes in the mevalonate pathway and the farnesyl pyrophosphate synthase is included in the nucleic acid construct, the synthesis of FPP would be facilitated, and in turn the biosynthesis of valencene would be facilitated.

In some embodiments, the nucleic acid construct is a plasmid vector; preferably, the plasmid vector is a eukaryotic expression vector.

In some embodiments, the nucleic acid construct includes a pRS426 plasmid backbone. The inventors have found that the pRS426 plasmid backbone includes AmpR screening marker suitable for Escherichia coli, URA3 screening marker suitable for Saccharomyces cerevisiae, and replicon suitable for Escherichia coli and multiple copy replicon suitable for Saccharomyces cerevisiae, and the use of the pRS426 plasmid backbone facilitates the maintenance of high copies of the plasmid including the gene of interest after introduction into Saccharomyces cerevisiae.

In some embodiments, a mutation is present in the pRS426 plasmid backbone, wherein the mutation eliminates the cleavage site BsaI in the pRS426 plasmid backbone, thereby allowing Bsal to be used as a restriction enzyme when constructing vectors using the Goldengate method.

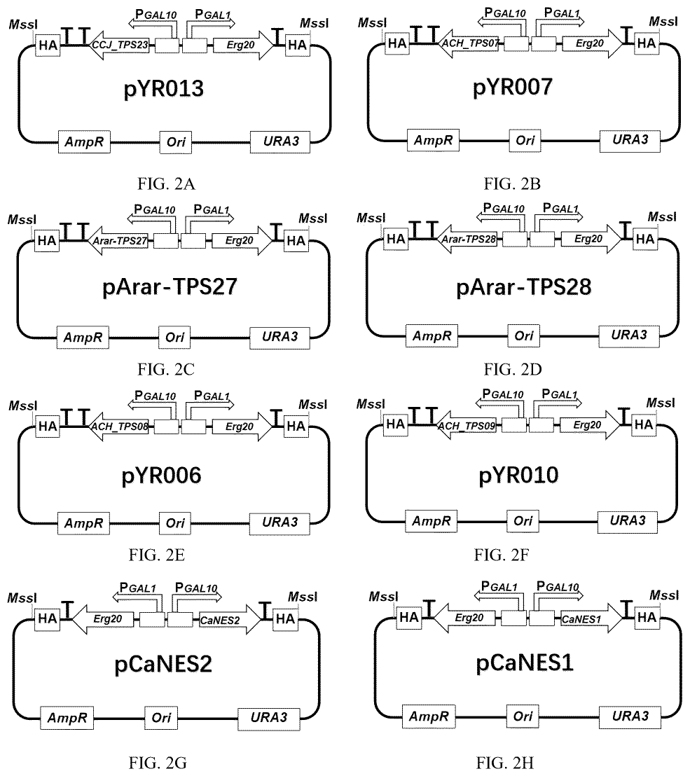

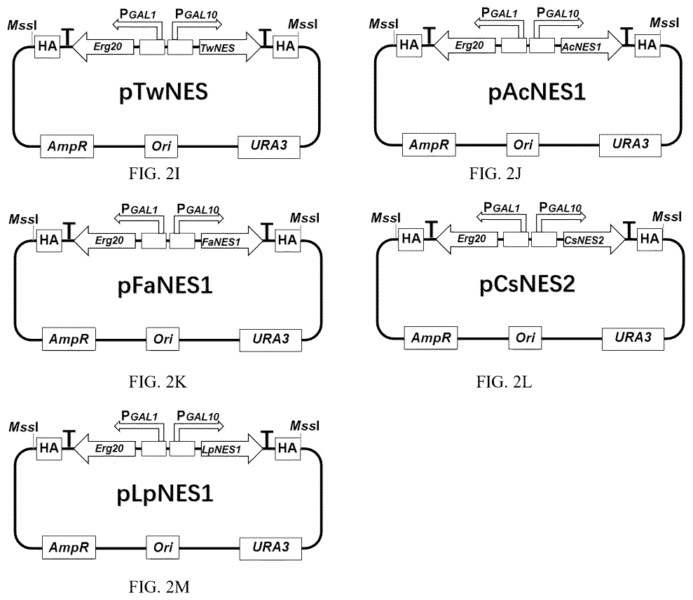

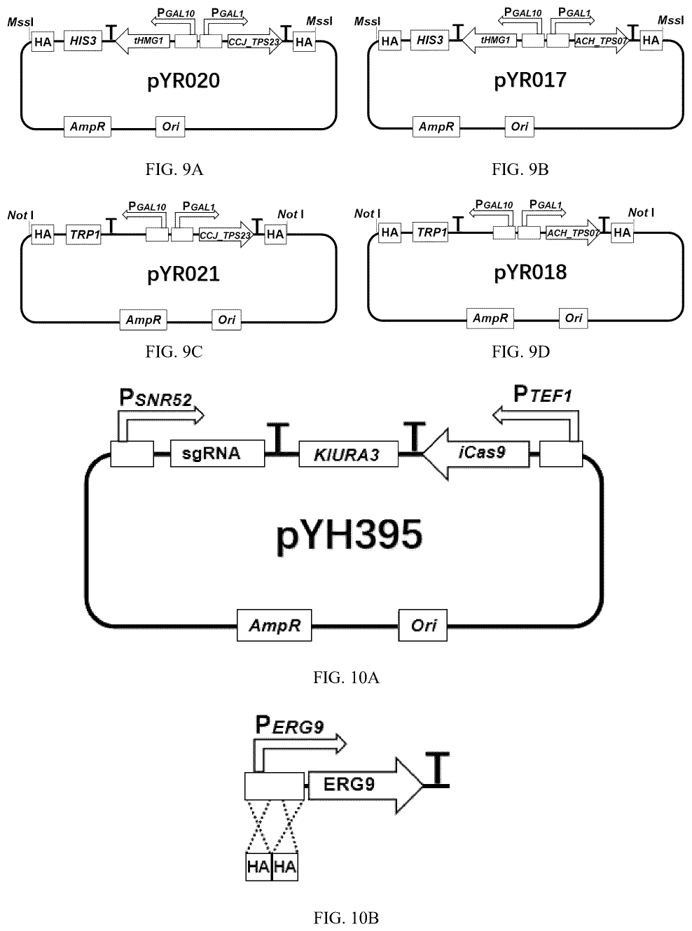

In some embodiments, the nucleic acid construct is at least one of plasmid vectors pYR006, pYR007, pYR010, pYR013, pAra-TPS27, pAra-TPS28, pYR017, pYR018, pYR020, and pYR021; and the construction schematic diagram of the plasmid vectors is as shown in FIGS. 2A-2F or FIGS. 9A-9D.

In some embodiments, the plasmid vectors can be directly introduced into the host cell, or fragments of the gene of interest including the insertion elements can be obtained by enzyme digestion of the plasmid vector, and the gene fragments can be further integrated into the genome of the host cell.

The fourth aspect of the present application provides a recombinant strain including the polynucleotide molecule provided in the second aspect of the present application, or the nucleic acid construct provided in the third aspect of the present application.

In some embodiments, the polynucleotide molecule is integrated into the genome of the host cell; preferably, the host cell is a eukaryotic cell; more preferably is Saccharomyces cerevisiae.

In some embodiments, a nucleic acid construct including a nucleotide sequence encoding the nerolidol synthase may be directly included in the recombinant strain, e.g., the nucleic acid construct is present alone in a host cell in the form of a plasmid, expressing the nerolidol synthase.

In other embodiments, the polynucleotide molecule is integrated into the genome of the host cell. The polynucleotide molecule is integrated into the genome of the host cell, which is beneficial to the long-term stable expression of the target gene, so as to obtain recombinant strain capable of stable inheritance.

A person skilled in the art can integrate polynucleotide molecules into the genome of the host cell by conventional methods, and is not limited herein by the present application. For example, the target gene can be connected between two insertion elements, and the target gene can be inserted into the genome of the host cell through the insertion element. For example, the insertion element can be the left and right homologous arms of Leu2, the left and right homologous arms of Ura3, the left and right homologous arms of YPRCdelta15, and the homologous arms of different genes can integrate the gene of interest into different locations in the host cell genome. The inventors have found that the recombinant strain of the present application can be obtained by inserting the target gene into each site that does not interfere with the normal physiological metabolism of the host cell.

In some embodiments, the copy number of the polynucleotide molecule is 1-3 in the genome of the recombinant strain.

In some embodiments, the recombinant strain is capable of endogenously and/or exogenously expressing at least one of acetoacetyl coenzyme A thiolase, hydroxy-methylglutaryl-CoA synthase, hydroxy-methylglutaryl-CoA reductase, mevalonate kinase, mevalonate-5-phosphate kinase, mevalonate pyrophosphate decarboxylase, isoprene pyrophosphate isomerase and farnesyl pyrophosphate synthase.

In some embodiments, the copy numbers of the acetoacetyl coenzyme A thiolase, hydroxy- methylglutaryl-CoA synthase, hydroxy-methylglutaryl-CoA reductase, mevalonate kinase, mevalonate-5-phosphate kinase, mevalonate pyrophosphate decarboxylase, isoprene pyrophosphate isomerase and farnesyl pyrophosphate synthase in the genome of the recombinant strain are 2, 2, 4, 2, 2, 2, 2, and 2, respectively.

The inventors have found that Saccharomyces cerevisiae can endogenously synthesize FPP, and therefore in some preferred embodiments, the use of Saccharomyces cerevisiae as host cell facilitates obtaining recombinant strains that efficiently synthesize nerolidol.

In some embodiments, the recombinant strain further includes knockout or downregulation of at least one of the genes encoding FPP hydrolase DPP1, FPP hydrolase LPP1, citrate synthase, malate synthase, or squalene synthase. The inventors have found that FPP hydrolase DPP1 (diacylglycerol pyrophosphate phosphatase 1) and FPP hydrolase LPP1 (lipid phosphate phosphatase 1) have the ability to hydrolyze FPP to produce farnesol, and citrate synthase (CIT2) and malate synthase (MLS1) can consume acetyl coenzyme A; squalene synthase (ERG9) synthesizes squalene with FPP as substrate, and these enzymes competitively consume substrates required for nerolidol biosynthesis. Therefore, knockout or downregulation of the encoding genes of these enzymes reduces the expression of these enzymes in recombinant strains, which is beneficial to improving the efficient synthesis of nerolidol in recombinant strains.

The downregulation as described in the present application has its general meaning, and can be understood as inhibition of gene expression in the present application, resulting in a decreased amount of expression of the protein regulated by the gene.

The knockout as described in the present application has its general meaning and refers to inactivation or deletion of a specific gene through a certain pathway, thereby the protein encoded by which is reduced in expressing or not expressing.

The fifth aspect of the present application provides the use of nerolidol synthase in the first aspect of the present application, the polynucleotide molecule in the second aspect of the present application, the nucleic acid construct in the third aspect of the present application or the recombinant strain in the fourth aspect of the present application in the production of nerolidol.

The sixth aspect of the present application provides a method for preparing nerolidol, including biosynthesizing nerolidol using the recombinant strain in the fourth aspect of the present application.

In some embodiments, the recombinant strain is capable of endogenously and/or exogenously expressing at least one of acetoacetyl coenzyme A thiolase, hydroxy-methylglutaryl-CoA synthase, hydroxy-methylglutaryl-CoA reductase, mevalonate kinase, mevalonate-5-phosphate kinase, mevalonate pyrophosphate decarboxylase, isoprene pyrophosphate isomerase and farnesyl pyrophosphate synthase.

The inventors have found that introducing the target gene into a host cell capable of endogenously expressing at least one of enzymes selected from the enzymes in the mevalonate pathway and farnesene pyrophosphate synthase facilitates further improvement of nerolidol production.

In some embodiments, at least one nucleotide sequence encoding acetoacetyl coenzyme A thiolase, hydroxy-methylglutaryl-CoA synthase, hydroxy-methylglutaryl-CoA reductase, mevalonate kinase, mevalonate-5-phosphate kinase, mevalonate pyrophosphate decarboxylase, isoprene pyrophosphate isomerase and farnesyl pyrophosphate synthase can be further linked into the nucleic acid construct, so that the recombinant strain can efficiently synthesize FPP, thereby contributing to further improvement of the yield of nerolidol.

In some embodiments, the copy numbers of acetoacetyl coenzyme A thiolase, hydroxy-methylglutaryl-CoA synthase, hydroxy-methylglutaryl-CoA reductase, mevalonate kinase, mevalonate-5-phosphate kinase, mevalonate pyrophosphate decarboxylase, isoprene pyrophosphate isomerase and farnesyl pyrophosphate synthase in the genome of the recombinant strain are 2, 2, 4, 2, 2, 2, 2, and 2, respectively.

In some embodiments, further includes knockout or downregulation of at least one of the genes encoding FPP hydrolase DPP1, FPP hydrolase LPP1, citrate synthase, malate synthase, or squalene synthase.

In some embodiments, when the nucleic acid construct used includes an inducible promoter, it may further include knockout of transcription inhibitor of the recombinant strain, for example, when GAL (galactose-inducible) promoter is used, transcription inhibitor GAL80 may be knocked out, and knockout of transcription inhibitor enable the recombinant strain to autonomously express the target gene without the need of an inducer, and reduces fermentation cost.

The nerolidol synthase and use thereof of the present application will be described by specific examples below. The following examples are used to further illustrate the present disclosure, but should not be construed as limiting the scope of the present disclosure. The plasmids involved in the following examples are all well-known to those skilled in the art. If specific techniques or conditions are not indicated in the examples, the test is carried out according to the techniques or conditions described in the literature in the art or according to the product specification. Reagents or instruments used without indicating the manufacturer are commercially available conventional products.

### Example 1 Functional Edentification of Nerolidol Synthase in Tanacetum cinerariifolium

### 1.1 Screening of Potential Nerolidol Synthetic Genes From Tanacetum cinerariifolium

RNA was extracted from samples taken from different tissues of Tanacetum cinerariifolium at different stages, and the second and third generation transcriptome sequencing was carried out; Tanacetum cinerariifolium transcriptome protein sequences including both Pfam domains PF01397 and PF03936 of terpene synthase were searched from the second and third generation transcriptome data, and 166 potential protein sequences were obtained. CD-Hit was used to conduct clustering to the found protein sequences to eliminate redundancy, and sequences with more than 90% sequence similarity were defined as a same class, results in a total of 33 classes were obtained; in each class, according to sequence integrity, protein sequences with a length greater than 500 were selected as candidate genes, and the genes with the highest expression level were further selected as test genes, a total of 24 genes to be verified were obtained, and the genes were named from CCJ-TPS01 to CCJ-TPS24.

### 1.2 Identification of Nerolidol Synthase and its Gene in Tanacetum cinerariifolium

### 1.2.1 Construction of Yeast Expression Universal Vector

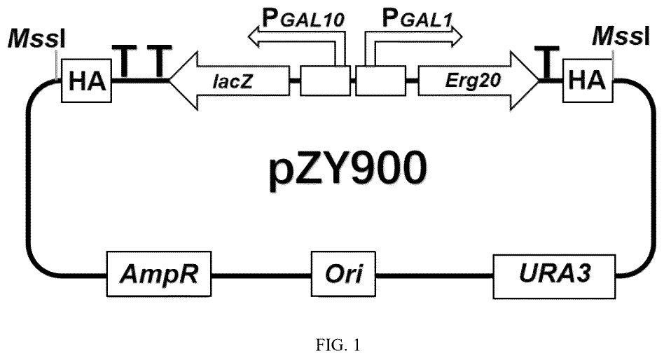

Specific construction process of plasmid pZY900: using the genome of Saccharomyces cerevisiae S288c (extraction method see: Xiaowei Li. Engineering Acetyl-CoA Pathway to Construct Efficient Biosynthesis Platform of Saccharomyces Cerevisiae [D]. Wuhan University, 2015. 2.3.6 Yeast genome DNA extraction method) as the template, amplify with primers 900-1F/1R, 900-2F/2R, 900-6F/6R and 900-7F/7R to obtain fragments 9001 (left homologous arm of Leu2), 9002 (terminator tTDH2), 9006 (gene ERG20 and terminator tERG20), 9007 (right homologous arm of Leu2) respectively; using the genome of Saccharomyces cerevisiae CEN.PK2-1D (extraction method see: Xiaowei Li. Engineering Acetyl-CoA Pathway to Construct Efficient Biosynthesis Platform of Saccharomyces Cerevisiae [D]. Wuhan University, 2015. 2.3.6 Yeast genome DNA extraction method) as the template, amplify with primers 900-3F/3R and 900-5F/5R to obtain fragments 9003 (terminator tCYC1) and 9005 (promoters pGAL1 and Pgal10) respectively; using pCAS (see literature Zhang, Yueping et al. “A gRNA-tRNA array for CRISPR-Cas9 based rapid multiplexed genome editing in Saccharomyces cerevisiae.” Nature communications vol. 10,1 1053. 5 Mar. 2019, doi:10.1038/s41467-019-09005-3) as the template, amplify with primers 900-4F/4R to obtain fragments 9004 (nonsense gene lac Z, used for replacement of target gene); using pRS426 as the template, amplify with primers 900-8F/8R, 900-9F/9R, 900-10F/10R to obtain plasmid backbone (MssI restriction site, and screening markers (AmpR, URA3, etc.) introduced). pZY900 was constructed by DNA assembly (also known as Yeast assembly, Xiaowei Li. Engineering Acetyl-CoA Pathway to Construct Efficient Biosynthesis Platform of Saccharomyces Cerevisiae [D]. Wuhan University) in Saccharomyces cerevisiae, and then amplified in Escherichia coli. pZY900 was obtained after verified by enzyme digestion and sequencing. A schematic diagram of the construction of plasmid pZY900 is shown in FIG. 1, in which fragments 9001 (H A), 9002 (T), 9003 (T), 9004, 9005, 9006 and 9007 (HA) are sequentially ligated from left to right, and the rest is derived from plasmid backbone of pRS426.

The sequences of the primers used to construct plasmid pZY900 are shown in Table 1 below:

TABLE 1

| primer | Sequence (5′-3′) |
|---|---|
| 900-1F | actaaagggaacaaaagctggagctctagtagtttaaacataacgagaacacacagggg (SEQ ID NO: |
| | 13) |
| 900-1R | cattaaagtaacttaaggagttaaatttaagcaaggattttcttaacttcttc (SEQ ID NO: 14) |
| 900-2F | gaagttaagaaaatccttgcttaaatttaactccttaagttactttaatgatttag (SEQ ID NO: 15) |
| 900-2R | tcgaaggctttaatttgcgcgaaaagccaattagtgtgata (SEQ ID NO: 16) |
| 900-3F | tagtatcacactaattggcttttegcgcaaattaaagccttegagc (SEQ ID NO: 17) |
| 900-3R | gggacgcgccctgtagcggctgaggtctcaacaggccccttttcctttg (SEQ ID NO: 18) |
| 900-4F | catgatatcgacaaaggaaaaggggcctgttgagacctcagccgctacagggcgc (SEQ ID NO: 19) |
| 900-4R | gaatttttgaaaattcaatataaatgtgagaccaccatgattacgccaagcg (SEQ ID NO: 20) |
| 900-5F | taatcatggtggtctcacatttatattgaattttcaaaaattcttactttttttttg (SEQ ID NO: 21) |
| 900-5R | atctctctctcctaatttctttttctgaagccattatagttttttctccttgacgttaaagt (SEQ ID NO: 22) |
| 900-6F | ttaacgtcaaggagaaaaaactataatggcttcagaaaaagaaattagga (SEQ ID NO: 23) |
| 900-6R | atgtacaaatatcataaaaaaagagaatctttttaaaaaaaatccttggactagtcacg (SEQ ID NO: 24) |
| 900-7F | actagtccaaggattttttttaaaaagattctctttttttatgatatttgtacataaac (SEQ ID NO: 25) |
| 900-7R | gcgccattcgccattcaggctgcgcaactgttgtttaaacgacaacgaccaagctcaca (SEQ ID NO: 26) |
| 900-8F | gatgtgagcttggtcgttgtcgtttaaacaacagttgcgcagcctgaatg (SEQ ID NO: 27) |
| 900-8R | tcaacagtatagaaccgtggatgatgtggtttctacaggatctgacattattattgttg (SEQ ID NO: 28) |
| 900-9F | atagtcctcttccaacaataataatgtcagatcctgtagaaaccacatcatccacggtt (SEQ ID NO: 29) |
| 900-9R | agggcttaccatctggccccagtgctgcaatgataccgcgcgacccacgctcaceggct (SEQ ID NO: 30) |
| 900-10F | tgataaatctggagccggtgagcgtgggtcgcgcggtatcattgcagcactggggccag (SEQ ID NO: 31) |
| 900-10R | cgatagcgcccctgtgtgttctcgttatgtttaaactactagagctccagcttttgttc (SEQ ID NO: 32) |

### 1.2.2 Construction of Yeast Expression Vector

Primers were designed to connect the gene to be verified onto a universal vector. Here we demonstrate the validation process of CCJ_TPS23, the gene in Tanacetum cinerariifolium that was finally validated to have nerolidol synthase activity.

CCJ_TPS23-F/R was designed as a specific gene primer pair. cDNA of Tanacetum cinerariifolium was (RNA was extracted from Tanacetum cinerariifolium ovule tissue by RNAprep Pure Plant Plus Kit (Cat No. DP441) of TIANGEN Company, cDNA was obtained by reverse transcription of RNA by HiScript II 1st Strand cDNA Synthesis Kit (+gDNA wiper) (Cat. No. R212) of Vazyme) used as the template, and CCJ_TPS23 gene fragment was amplified by PCR using Phanta high fidelity enzyme of Vazyme Company. After gel recovery was carried out by using Tiangen gel recovery kit, it was ligated into Bsal cleaved yeast expression vector pZY900 by methods for homologous recombination using homologous recombination kit of Yeasen Company. After sequencing confirmation, the yeast expression vector including this gene was obtained and named pYR013. The plasmid construction schematic diagram is shown in pYR013 in FIG. 2A, in which the lac Z gene in pZY900 is replaced by CCJ_TPS23 gene.

CCJ_TPS23-F/R primer sequences are shown in Table 2 below:

TABLE 2

| primer | Sequence (5′-3′) |
|---|---|
| CCJ_TPS23-F | acaaaggaaaaggggcctgtttaaaacatggagtttatatattccttgatg (SEQ ID NO: 33) |
| CCJ_TPS23-R | tttttgaaaattcaatataaatgatcatcaacaattgtgttacac (SEQ ID NO: 34) |

### 1.2.3 Strain Construction

Plasmid pYR013 was introduced by lithium acetate method (Xiaowei Li. Engineering Acetyl-CoA Pathway to Construct Efficient Biosynthesis Platform of Saccharomyces Cerevisiae [D]. Wuhan University, 2015. 2.3.14 Saccharomyces cerevisiae LiAc transformation) into strain JCR27 (for the construction of yeast strain JCR27 see literature Siemon, Thomas et al. “Semisynthesis of Plant-Derived Englerin A Enabled by Microbe Engineering of Guaia-6,10(14)-diene as Building Block.” Journal of the American Chemical Society vol. 142,6(2020): 2760-2765. doi:10.1021/jacs.9b12940) and the engineered strain was named CCJ-1.

Plasmid pZY900 was introduced into strain JCR27 using the same method as a control strain, and was named S900.

### 1.2.4 Strain Fermentation and Product Identification

CCJ-1 and S900 were seeded respectively into SC-URA liquid medium (Xiaowei Li. Engineering Acetyl-CoA Pathway to Construct Efficient Biosynthesis Platform of Saccharomyces Cerevisiae [D]. Wuhan University, 2015, uracil-deficient medium), and incubated under 30° C., 200 rpm with shaking overnight; transferred with an initial OD600=0.1 into 45 ml of YPDHG liquid medium (20 g/L tryptone, 10 g/L yeast extract, 10 g/L glucose, 10 g/L galactose) on the next day, followed by incubation for 72 hours under 30° C., 200 rpm shaking, with additional 5 ml isopropyl myristate. The oil layer was collected and diluted with n-hexane to a suitable concentration, and the product was detected using GC-MS.

TRACE GC ULTRA gas chromatography of Thermo Fisher Scientific equipped with AS 3000 autosampler, split/splitless injector, and TSQ QUANTUM XLS MS equipped with triple quadrupole detector.

TR-5 MS column (30 m×0.25 mm×0.25 um) was used as the chromatographic column. The carrier gas is high purity helium at a flow rate of 1 mL/min. Acetone was used for needle wash. Injection volume is 1 uL, split ratio is 50. The temperature of injection port is 240° C., and the temperature of ion transport tube is 270° C.

Detection procedure: Initial column temperature is 50° C., hold for 1 min; raise temperature to 280° C. at 15° C./min, hold for 1 min; raise temperature to 300° C. at 20° C./min, hold for 2 min.

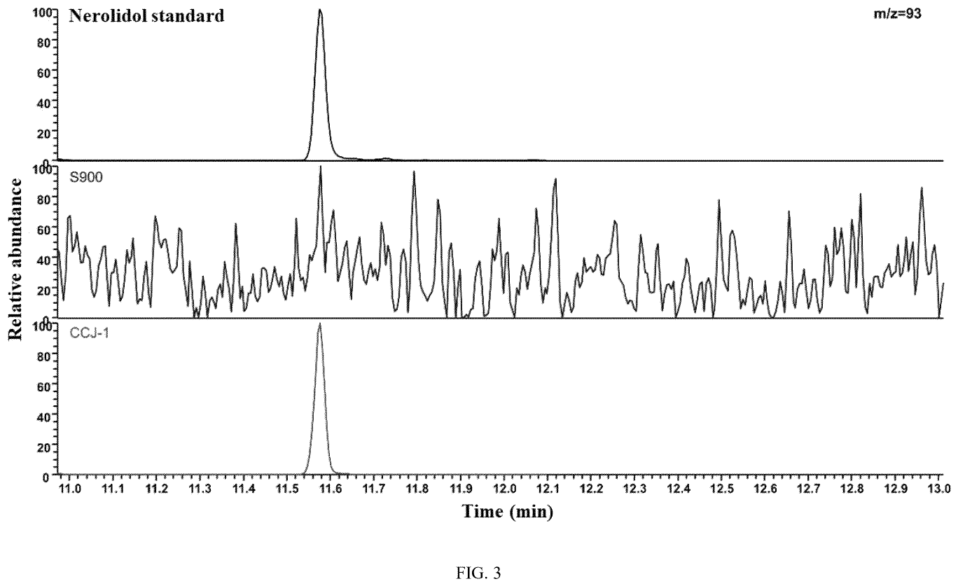

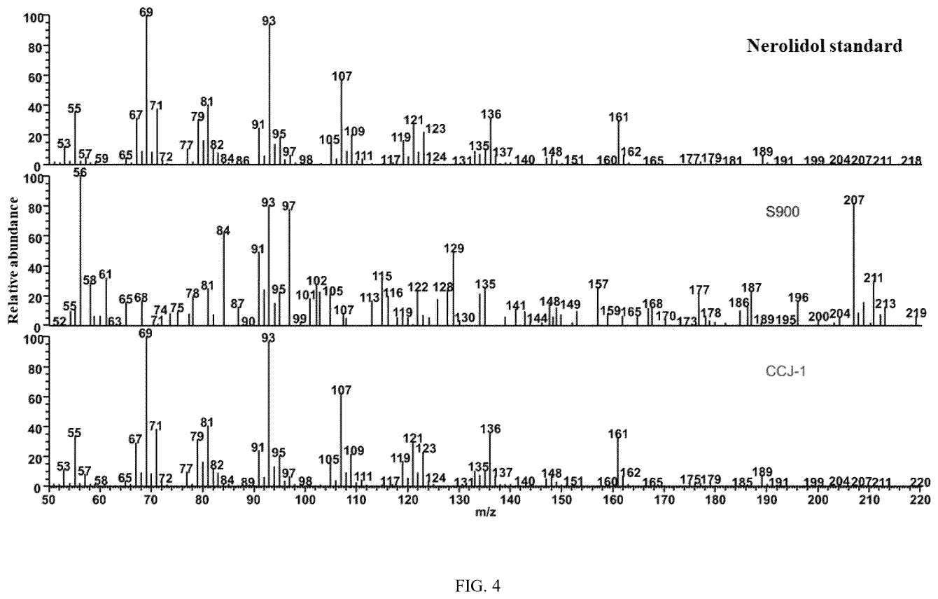

The extracted ion flow chromatogram of nerolidol characteristic ion m/z=93 in CCJ-1 and S900 fermentation products is shown in FIG. 3, and the mass spectrum of nerolidol is shown in FIG. 4. By comparing the chromatogram retention time and mass spectrum fragments with nerolidol standard, it can be determined that nerolidol can be synthesized in strain CCJ-1, but not in strain S900. These results indicate that the protein CCJ_TPS23 gene encodes is a nerolidol synthase.

Plasmid pYR013 was digested with MssI (see the instruction manual of MssI enzyme for conditions of enzyme digestion system) to obtain linearized fragments, which were integrated into strain JCR27 by lithium acetate method to obtain strain CCJ-2. The strain was seeded into YPD liquid medium and incubated under 30° C., 200 rpm with shaking overnight; transferred with an initial OD600-0.1 into 45 ml of YPDHG liquid medium on the next day, followed by incubation for 72 hours under 30° C., 200 rpm shaking, with additional 5 ml isopropyl myristate. The oil layer was collected and the product was detected using GC-MS, the shake flask fermentation yield of nerolidol of strain CCJ-2 was 271 mg/L.

### Example 2 Functional Identification of Nerolidol Synthetase in Astilbe chinensis

### 2.1 Screening of Potential Nerolidol Synthetic Genes from Astilbe chinensis

According to the same method, RNA was extracted from samples taken from different tissues of Astilbe chinensis at different stages, and the second and third generation transcriptome sequencing was carried out; Astilbe chinensis transcriptome protein sequences including both Pfam domains PF01397 and PF03936 of terpene synthase were searched from the second and third generation transcriptome data, and 89 potential protein sequences were obtained. CD-Hit was used to conduct clustering to the found protein sequences to eliminate redundancy, and sequences with more than 90% sequence similarity were defined as a same class, results in a total of 25 classes were obtained; in each class, according to sequence integrity, protein sequences with a length greater than 500 were selected as candidate genes, and the genes with the highest expression level were further selected as test genes, a total of 17 genes to be verified were obtained, and the genes were named from ACH-TPS01 to ACH-TPS17.

### 2.2 Identification of Nerolidol Synthase and its Gene in Astilbe chinensis

### 2.2.1 Construction of Yeast Expression Vector

Primers were designed to construct the gene to be verified onto a universal vector. Here we demonstrate the validation process of genes ACH_TPS07, ACH_TPS08, and ACH_TPS09 that were finally validated to have nerolidol synthase activity in Astilbe chinensis.

ACH_TPS07-F/R, ACH_TPS08-F/R, and ACH_TPS09-F/R were designed as specific gene primer pairs. cDNA of Astilbe chinensis was (RNA was extracted from Astilbe chinensis ovule tissue by RNAprep Pure Plant Plus Kit (Cat No. DP441) of TIANGEN Company, cDNA was obtained by reverse transcription of RNA by HiScript II 1st Strand cDNA Synthesis Kit (+gDNA wiper) (Cat. No. R212) of Vazyme) used as the template, and ACH_TPS07, ACH_TPS08, and ACH_TPS09 gene fragments were amplified by PCR using Phanta high fidelity enzyme of Vazyme Company. After gel recovery was carried out by using Tiangen gel recovery kit, it was ligated into BsaI cleaved yeast expression vector pZY900 by methods for homologous recombination using homologous recombination kit of Yeasen Company. After sequencing confirmation, the yeast expression vector including this gene was obtained and named pYR013. The plasmid construction schematic diagram of plasmids pYR007, pYR006 and pYR010 are shown in FIG. 2B and FIGS. 2E-2F as pYR007, pYR006 and pYR010, in which the lac Z gene in pZY900 is replaced by genes ACH_TPS07, ACH_TPS08 and ACH_TPS09, respectively.

Primer sequences are shown in Table 3 below.

TABLE 3

| Plasmid | Primer | Sequence (5′-3′) |
|---|---|---|
| pYR007 | ACH TPS07-F | acaaaggaaaaggggcctgtttaaaacaaacattgtatatgctcctcaag |
| | | (SEQ ID NO: 35) |
| | ACH TPS07-R | tttttgaaaattcaatataaatggcaccccctccttcc (SEQ ID NO: 36) |
| pYR006 | ACH TPS08-F | acaaaggaaaaggggcctgtctatgtaacgtttatcatatttaaaacaaacattg (SEQ ID |
| | | NO: 37) |
| | ACH TPS08-R | tttttgaaaattcaatataaatggcaccccctccttcc (SEQ ID NO: 38) |
| pYR010 | ACH TPS09-F | acaaaggaaaaggggcctgtctatgtaacgtttatcatatttaaaacaaacattg (SEQ ID |
| | | NO: 39) |
| | ACH TPS09-R | tttttgaaaattcaatataaatggcaccccctccttcc (SEQ ID NO: 40) |

### 2.2.2 Strain Construction

According to the same method in 1.2.3 of Example 1, plasmid pYR007 was introduced into strain JCR27 and the engineered strain was named LXF-1; plasmid pYR006 was introduced into strain JCR27 and the engineered strain was named LXF-1-1; plasmid pYR010 was introduced into strain JCR27 and the engineered strain was named LXF-1-2. Plasmid pZY900 was introduced into strain JCR27 as a control strain, and was named S900.

### 2.2.3 Strain Fermentation and Product Identification

Strains LXF-1, LXF-1-1, LXF-1-2 and S900 were seeded respectively into SC-URA liquid medium, and incubated under 30° C., 200 rpm with shaking overnight; transferred with an initial OD600-0.1 into 45 ml of YPDHG liquid medium on the next day, followed by incubation for 72 hours under 30° C., 200 rpm shaking, with additional 5 ml isopropyl myristate. The oil layer was collected and the product was detected using GC-MS, under the same conditions as in 1.2.4 of Example 1.

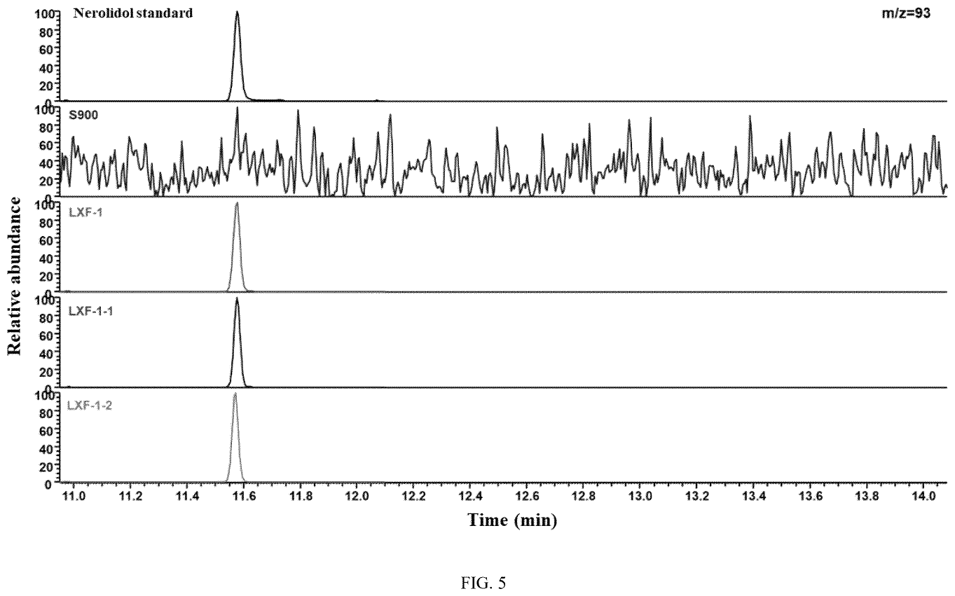

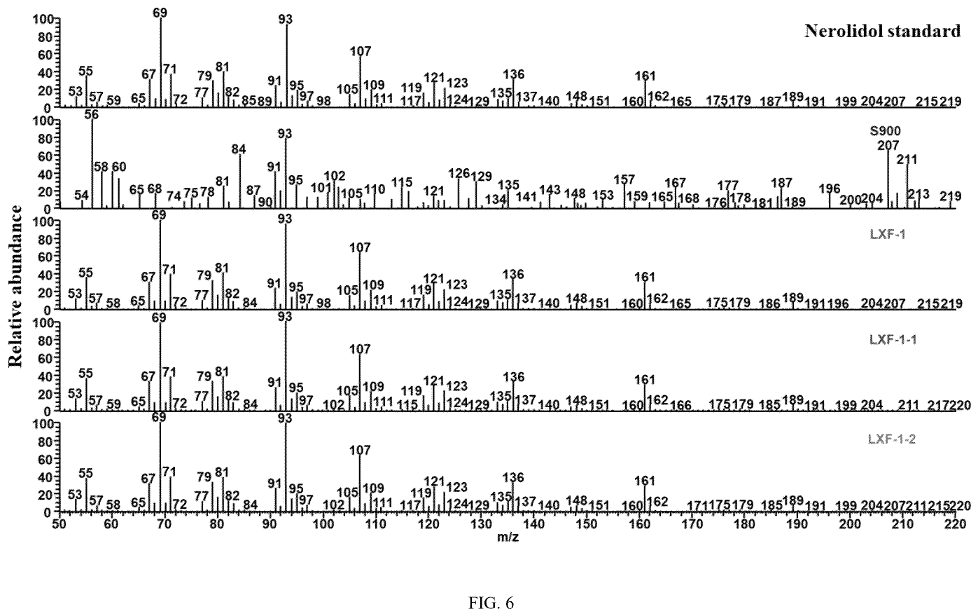

The extracted ion flow chromatogram of nerolidol characteristic ion m/z=93 in LXF-1, LXF-1-1, LXF-1-2 and S900 fermentation products is shown in FIG. 5, and the mass spectrum of nerolidol is shown in FIG. 6. By comparing the chromatogram retention time and mass spectrum fragments with nerolidol standard, it can be determined that nerolidol can be synthesized in strain LXF-1, LXF-1-1, LXF-1-2, but not in strain S900. These results indicate that the proteins ACH_TPS07, ACH_TPS08 and ACH_TPS09 gene encodes are nerolidol synthases.

Plasmid pYR007, pYR006 and pYR010 were digested respectively with MssI (see the instruction manual of MssI enzyme for conditions of enzyme digestion system) to obtain linearized fragments, which were integrated into strain JCR27 by lithium acetate method to obtain strains LXF-2, LXF-2-6 and LXF-2-10. The strains were seeded into YPD liquid medium and incubated under 30° C., 200 rpm with shaking overnight; transferred with a initial OD600=0.1 into 45 ml of YPDHG liquid medium on the next day, followed by incubation for 72 hours under 30° C., 200 rpm shaking, with additional 5 ml isopropyl myristate. The oil layer was collected and the product was detected using GC-MS, the shake flask fermentation yields of nerolidol of strains LXF-2, LXF-2-6 and LXF-2-10 were 269 mg/L, 240 mg/L and 221 mg/L respectively.

### Example 3 Functional Identification of Nerolidol Synthetase Artemisia argyi

### 3.1 Screening of Potential Nerolidol Synthetic Genes From Artemisia argyi

Artemisia argyi transcriptome protein sequences containing both Pfam domains PF01397 and PF03936 of terpene synthase were searched from Artemisia argyi transcriptome data (https://www.ncbi.nlm.nih.gov/bioproject/PRJNA722539), and 167 potential protein sequences were obtained. CD-Hit was used to conduct clustering to the found protein sequences to eliminate redundancy, and sequences with more than 90% sequence similarity were defined as a same class, results in a total of 47 classes were obtained; in each class, according to sequence integrity, protein sequences with a length greater than 500 were selected as candidate genes, and the genes with the highest expression level were further selected as test genes, a total of 29 genes to be verified were obtained, and the genes were named from Arar-TPS01 to Arar-TPS29.

### 3.2 Identification of Nerolidol Synthase and its Gene in Artemisia argyi

### 3.2.1 Construction of Yeast Expression Vector

Primers were designed to construct the gene to be verified onto a universal vector. Here we demonstrate the validation process of genes Arar-TPS27 and Arar-TPS28 that were finally validated to have nerolidol synthase activity in Artemisia argyi. The genes Arar-TPS27 and Arar-TPS28 derived from Artemisia argyi were directly synthesized according to the codon optimization of Saccharomyces cerevisiae, and the sequences are shown in SEQ ID NO: 7 and SEQ ID NO: 8.

Arar-TPS 27-F/R and Arar-TPS 28-F/R were designed as specific gene primer pairs, gene fragments obtained by PCR amplification was used as the template, and Arar-TPS27 and Arar-TPS28 gene fragments were amplified by PCR using Phanta high fidelity enzyme of Vazyme Company. After gel recovery was carried out by using Tiangen gel recovery kit, it was ligated into Bsal cleaved yeast expression vector pZY900 by methods for homologous recombination using homologous recombination kit of Yeasen Company. After sequencing confirmation, the yeast expression vector including this gene was obtained and named pArar-TPS27 and pArar-TPS28. The plasmid construction schematic diagrams of plasmids pArar-TPS27 and pArar-TPS28 are shown in FIGS. 2C-2D as pArar-TPS27 and pArar-TPS28, in which the lac Z gene in pZY900 is replaced by genes pArar-TPS27 and pArar-TPS28 respectively.

Primer sequences are shown in Table 4 below.

TABLE 4

| Plasmid | Primer | Sequence (5′-3′) |
|---|---|---|
| pArar-TPS27 | Arar-TPS27-F | ggaaaaggggcctgtttacttatcgtcgtcatccttgtaatcgaacatggagttaatgtattcttc |
| | | (SEQ ID NO: 41) |
| | Arar-TPS27-R | gaatttttgaaaattcaatataaatgacaatctctgttactag (SEQ ID NO: 42) |
| pArar-TPS28 | Arar-TPS28-F | ggaaaaggggcctgtttacttatcgtcgtcatccttgtaatccatatcaatagccttgaagaaca |
| | | ag (SEQ ID NO: 43) |
| | Arar-TPS28-R | gaatttttgaaaattcaatataaatgtctatcaacatcttgcatg (SEQ ID NO: 44) |

### 3.2.2 Strain Construction

According to the same method in 1.2.3 of Example 1, plasmid pArar-TPS27 was introduced into strain JCR27, and the engineered strain was named AH-1; plasmid pArar-TPS28 was introduced into strain JCR27, and the engineered strain was named AH-2; plasmid pZY900 was introduced into strain JCR27 as a control strain, and was named S900.

3.2.3 Strain fermentation and product identification

Strains AH-1, AH-2, and S900 were seeded respectively into SC-URA liquid medium, and incubated under 30° C., 200 rpm with shaking overnight; transferred with an initial OD600=0.1 into 45 ml of YPDHG liquid medium on the next day, followed by incubation for 72 hours under 30° C., 200 rpm shaking, with additional 5 ml isopropyl myristate. The oil layer was collected and the product was detected using GC-MS, under the same conditions as in 1.2.4 of Example 1.

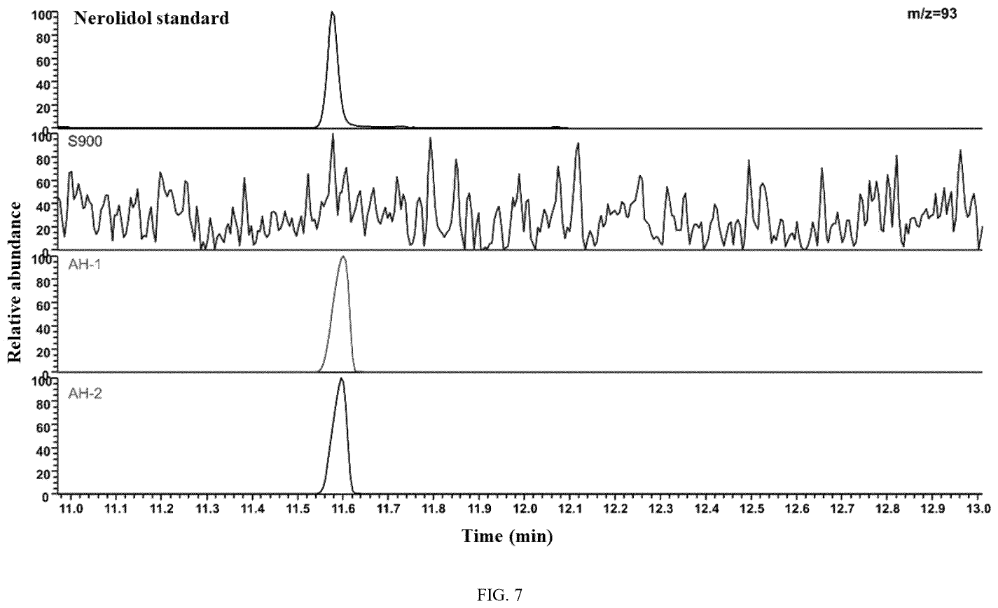

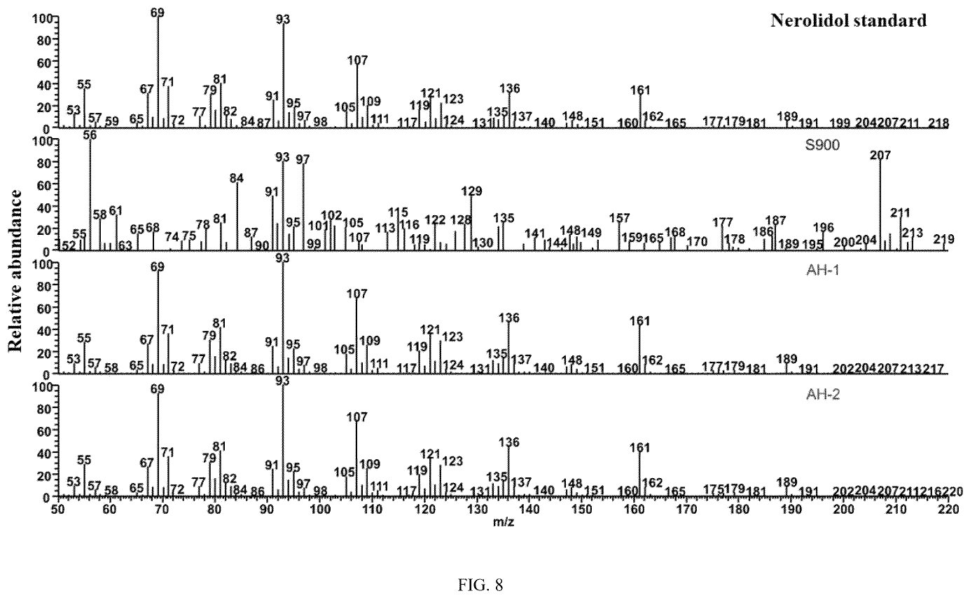

The extracted ion flow chromatogram of nerolidol characteristic ion m/z=93 in AH-1, AH-2 and S900 fermentation products is shown in FIG. 7, and the mass spectrum of nerolidol is shown in FIG. 8. By comparing the chromatogram retention time and mass spectrum fragments with nerolidol standard, it can be determined that nerolidol can be synthesized in strain AH-1 and AH-2, but not in strain S900. These results indicate that the proteins Arar-TPS27 and Arar-TPS28 gene encode are nerolidol synthases.

Plasmid pArar-TPS27 and pArar-TPS28 were digested respectively with MssI (see the instruction manual of MssI enzyme for conditions of enzyme digestion system) to obtain linearized fragments, which were integrated into strain JCR27 by lithium acetate method to obtain strains AH-27 and AH-28. The strains were seeded into YPD liquid medium and incubated under 30° C., 200 rpm with shaking overnight; transferred with an initial OD600=0.1 into 45 ml of YPDHG liquid medium on the next day, followed by incubation for 72 hours under 30° C., 200 rpm shaking, with additional 5 ml isopropyl myristate. The oil layer was collected and the product was detected using GC-MS, the shake flask fermentation yields of nerolidol of strains AH-27 and AH-28 were 162 mg/L and 158 mg/L respectively.

### Comparative Example 1 Expression of an Existing Nerolidol Synthase Gene

### 1.1 Construction of Plasmid Including Nerolidol Synthase Gene From Celastrus angulatus (CaNES 2), Celastrus angulatus (CaNES 1), Tripterygium wilfordii (TwNES), Actinidia chinensis (AcNES 1), Fragaria x anassa (FaNES 1), Camellia sinensis (CsNES2), and Laggera pterodonta (LpNES1)

The nerolidol synthase from different sources used in this comparative example are nerolidol synthases widely studied in the field and recognized as having high yield.

The nucleotide sequence of nerolidol synthase from Celastrus angulatus after
Saccharomyces cerevisiae codon optimization is as follows (5′-3′), and the gene
is named CaNES2.
(SEQ ID NO: 99)
ATGGACTTTTTTGATTGTTCCAGATCTTCCATTATTCCATTGAAGGCTTCCTCCCAAATT
GTTCCAGCTGATTCTACTAACAAGTGGGGTACTGTCGAATCTAACCACAACTCTGCTC
CAACTACCCCATTAAACGACAGAATTTACGTTGAACACGCTCACATGGTCAAGGATTT
CAAGAGAATCATGAACGTCGCTGGTGAAGACCCATCTGAAGGTTTGGCTATCATCGAT
GCTGTCCAAAGATTGGTTGTCGACCATCATTTCCAAGAAGAAATTCACTCCATCCTAC
AAAAGCATAAGGAATTGGCTACCTCTACCACCACACACGGTGACTGTATGACCACCT
CTTTGAGATTCAGATTGTTGAGACAAGAAGGTTACTACGTCTCTGCTGATGTTTTCGA
AGGTCTAAAAGATGAAGAAGGTAAGTTCGACCAAAACTTGAGTGGTGACATCAAGG
GTTTGATGGCCTTGTACGAAGCTTCTCAATTCTCCATGGAAGGTGAAAACATCTTGGA
TGAAGCCAGAGACTACTCTGCCCGTTTATTGAATGCTTGTGTTACCCAATTGGGTCAC
GATCAAGCTCGTATTGTCGAACACACTCTGACTCACCCACACCACAAGTCTCTAGCC
AGATTCATGGCCAAGAACTTCTTGAGAGATTTCCATTGCACTAACGGTTGGATTGACG
ACTTGAAGAAGTTGGCCAAAGCTGACATCGACATGGCTCAATCCACTTTCCAAAACG
AAGTTGTTCAAATTTCTCAATGGTGGAAGGAATTAGGCTTAGCTGAAGAATTGAAATT
TGCTCGTGACCAACCAGTCAAATGGTACATCTGGACCATGACTTGTGACGAAGATCCA
TCTTTCTCCGAATTGAGAATCAACTTGACCAAGCCAATCTCTTTCGTCTACTTGATTGA
TGACATCTTCGATGTTTATGGTACCTTGCAAGAAGTCACTGATTTCACCGAAGCTGTT
CACAGATGGGACCACGACGCCATTGAACAATTGCCATACCACATGAAGATTTGTTTCA
AGGCTTTGGACGATATCACCAACGAAATCTCCTACAAGGTCTACAAGCAATACGGATG
GAACCCTTTAGACTCCCTCAGAAAGTCTTGGGGTAGATTGTGTAACGCTTTCCTAACT
GAAGCCAAGTGGTTCGCTTCTGGTCATTTGGCTAAGGCTGAAGAATATTTAGAAAAC
GGTATCATTTCCTCTGGTGTTCCTGTTGTTTTGTTGCACTTCTTTTTTCTTAGTGGTGAA
GGTGTTACTCAAAAGTCCGTTGAAACCATTGACAACACTCCAGACATTGTCTCCGCT
GCAGCCGCTATCTTGCGTTTATGGGATGATTTGGGTTCAGCTAAGGACGAAGACCAAG
ATGGTAAGGACGGTTCTTACTTGGCCTGTTACACTAATGAAAACCCAGGTTGTTCTTT
GGAAGATGCTGAGAAGCACGTTAAGTCCAAGATCTGTGATGAATGGAAATTGTTGAA
CAAGGAGTGTTTGTCTCAAAAGAACCCATTCTCTGGTTCCTTCCACAGAGCTTGCTTG
AATGTTGCTAGAATGGTTCCATTGATGTACGAATACGACAAGAACAGAAGATTGCCAA
GATTGGAAGAATTCTTGAAGAGTATCTTGATCAAATGA
The nucleotide sequence of nerolidol synthase from Celastrus angulatus after
Saccharomyces cerevisiae codon optimization is as follows (5′-3′), and the
gene is named CaNES1.
(SEQ ID NO: 100)
ATGAACTTGCCAATGACTCTAAGCTCCAAGTCCTTATTCACTTCTTCCAGAGCCTCTAT
CAGCTCTTCTTTCAAGACTATTTCCCAAATTGAAATTGCTAAAACTATCGCCAACTGTG
TCCCATTGATTCCAGAACACCACAAGTGGTCTATTCACGATGTTTACCACAAATTAAG
ATTTTCCAACACTACAACCCCACCATTTAAGCAATTGAATCATTTGACCAAGCCATGC
ACCCCAAACGACCACATTTCTAAGGAACACGAAAGAAAGGTCAAGGCTTTCAAGAG
GATTTTCAACATTGTTGGTGAAGACCCAATCGAATCCTTGGTCATCATTGACGCTGTC
CAACGTCTCGGTATTGACCATCACTTCCAAGAAGAAATTAACACCATTCTTCAAGAAT
TGTACTACACTGAAGCTAACAACGCTCACATCGGTGGTGGTGGTGGTTACTTCTCCCA
CTTGCACGAAGTTTCGCTAAGATTCAGATTGTTGCGTCAACAAGGTTGTTACGTTTCT
CCAGAAGTCTTTGACTGTTTCAAGGACAACAACGGTAAGTTCAAAGAAAACTTGGGT
GGTGACATCAACGGTTTGATGGCCTTATACGAAGCCTCCCATTTGTGTTTGGAAGGTG
AAAGTCTATTGCAAGAAGCTCGTGAATGTTCTGCCCAATTATTAAAGACCATCGTCAC
TAACTTGAATCACTCTCAAGCTACCATGGTCGAACACACTTTGTCTCATCCACACCAC
AAGTCTTTCTCTAAATTCACCGGTAAGAAGTACTTGTCTCACGGTTTCCAAGATACCA
ACAATGGCTGGTTGAACATCTTGAAGGGTTTGGCTAAGATTGATTTCGACATGGCTCA
ATCCACTTACCAAAAGGAAATTGTCCAAATCTCTAAATGGTGGGAAGAATTGGGTTTG
GCCAAGGAAATGTCCTTCGCCAGAGATCAACCATTAAAGTGGTACATCTGGTCTATGG
CTTGTATCACCGACCCATCCTTATCTGAAGAAAGAATTGACTTGACTAAGTCCATCTCA
TTCATATACTTGATTGATGATATCTTCGATTTGTACGGTACTCAAGACGAATTAACCTTG
TTTACCCAAGTTGTTAACAGATGGGACTACGATGCTAGTGAACAATTGCCTCATTACAT
GAAGATTTGTTTCAAGGCTTTGGACGATATTATCAACGAAATTTCTTTTAAGATCTACA
AGAAGCACGGATGGAACCCAGTTGAATCTTTGAGAAAGTCTTGGGGCAGACTATGCT
CTGCATTCTTGGTTGAAGGTAGATGGTTCGCTTCAGGTTATGTCCCAGCTTCCGAAGA
ATACTTGAAGAACGGTATCATCTCTTCTGGTGTTCATGTTGTCTGTGTTCAATTGTTCT
TCTTGTTGGGTAGACAAGATATAATTACTAAGCAATCTGTCGAAATCATTGACAACGAT
CCAGCCATCGTCTCCTCTACTGCTCAAATCTTGAGATTGTGGGATGACCTGGGTTCTG
CTAAGGATGAGGACCAAGATGGTCACGACGGTTCCTATTTAGACTACTATTTGAAAGA
GCACCAAGGTTGTTCCGTTAAGGATGCTGAAGAACACGTTATGTCTAAGATCTCTGAC
GCTTGGAAGCAATTGAACAAGGAATGTATGACGCCAACCCCATTCTCTCAATGTTTCA
TCAAGGCTACTTTGAACTTGGCTAGAATGGTTCCTTTGATGTACTCTTACGACAACAA
CCAAAGATTACCATCTTTGGAAGATAACGTTAAATCTATCTTGTCCATTGCTTTGTGA
The nucleotide sequence of nerolidol synthase from Tripterygium wilfordii after
Saccharomyces cerevisiae codon optimization is as follows (5′-3′), and the
gene is named TwNES.
(SEQ ID NO: 101)
ATGGCTTTCTTCGGTTCCTCTAGATCTTCAATTATTCCACTAAAGACTATTAGTCAAATT
GTTACTGCTGACTCCACCAACAAGAGAGGTACCGTTGATTCTAACCACAAGTCCGCT
CCAACCACTCCTTTGAACGACCGTATCTGCACCGAACACGCTCACAAGGTCAAGGAT
TTCAAGCAAATCATCAACATTGCTGGTGAAGATCCATCTGAAGGCTTGGCTATCATTG
ATGCTGTTCAAAGATTGGGTGTTGACCATCACTTCCAAGATGAAATTCACACTATCTT
GCAAAAGCATTACACCCTCGCTACCGGTACGACCACCCATGGTGACTGTATGATCACT
TCTTTGAGATTCAGATTACTTAGACAGGAAGGTTACTACGTTTCTGCCGATGTTTTCGA
AGGTTTAAAGGATGAAGAAGGTAAGTTCGACCAAAACTTGTCTGGTGACATCAAGGG
TTTAATGGCTTTGTACGAAGCCTCCCAATTGTCCATGGAAGGTGAAAACATCTTGGAC
GAAGCTAGAGACTACTCTTCCAGATTGTTGAACGAATGTGTTACCCAATTGGACCACG
ACCAAGCTAGAATCGTCGAACACACTTTGACCCACCCACACCACAAATCTCTAGCCC
GTTTCATGGCTAAGAACTTCTTAAGAGACTTCCACGGTACTAACGGTTGGATTGACGA
CTTGAAGAAGTTGGCCAAGGTTGACTTTGACATGGCCCAATCTACTTACCAAAAGGA
AGTCGTCCAAATCAGTCAATGGTGGAAGGAATTGGGTTTGGCTGAAGAATTGAAATT
CGCTAGAGATCAACCAGTCAAGTGGTACATCTGGACCACAACTTGTCACCAAGACCC
ATCTTTCTCGGAATTGCGTATTAACTTAACCAAGCCAATTTCTTTCGTCTACTTGATTGA
TGATATCTTTGATGTCTACGGTACCCCACAAGAAGTTACTGACTTCACTGAAGCTGTC
GATAGATGGGATCATGATGCCATCGACCAATTGCCATACTACATGAAGATCTGTTTCAA
GGCTTTAGATGACATAACCAACGAAATCTCTTACAAGGTTTACAAGCAACACGGTTG
GAACCCATTGGATTCATTGAGAAAATCTTGGGGTAGATTATGTAACGCTTTCTTGACTG
AAGCTCAATGGTTCGCTTCTGGTCACCTGCCAAAGGCCGAAGAATACTTGGAAAATG
GTATCATCAGCTCCGGTGTCCCAGTTGTTTTGCTGCATTTGTTCTTTTTGTTGGGTGAA
GGTGTCACTCAAAAATCCGTTGAAATGATTGACAACACTCCAGCTATTGTCTCTGCTG
CCGCTGCTATTTTGAGATTATGGGACGATTTGGGCTCTGCTAAGGACGAAGACCAAGA
TGGAAAGGATGGTTCTTATTTGGCATGTTACACTAATGAAAACCCAGGTTGTTCCTTG
GAAGACGCTGAAAAGCACGTTAAGTCTAAGATTTGTGATGAATGGAAACAATTAAAT
AAAGAATGTTTGTCCCAAAAGAACCCATTCTCTCCATCCTTTTTGAAGGCTTGCTTGA
ACGTTTCCAGAATGGTTCCTTTGATGTACGAATATGACGAAAACAGACGTTTGCCAAG
ATTGGAAGAATTCTTGAAATCCATCTTAATCAAGtaa
The nucleotide sequence of nerolidol synthase from Actinidia chinensis after
Saccharomyces cerevisiae codon optimization is as follows (5′-3′), and the
gene is named AcNES1.
(SEQ ID NO: 102)
ATGGCCACTGCCGCTGGTCCAATTGCCACCAACAACTCCCCACAAAACTCTAACGCT
TACAGAACCCCAATCGCTCCATCTGTTCCAATCACCCACAAGTGGTCTATTGCTGAAG
ACTTGACTTGTATCTCCAACCCATCCAAGCATAACAACCCTCAAACCGGTTACAGATC
TTTTTCTGATGAATTATACGTTAAGTACGAAGAAAAGTTGGAAGATGTCAGAAAGGCT
TTGAGAGAAGTTGAAGAAAACCCATTGGAAGGTTTAGTTATGATTGACGCTTTGCAA
AGATTGGGAATTGATTATCACTTCAGAGGTGAAATCGGTGCTTTCTTGCAGAAGCAAC
AAATCATTTCTTCTACTCCAGATGGTTACCCAGAACACGGTTTATATGAAGTCTCCACT
TTATTCAGATTCTTGAGACAAGAAGGTCACAACGTTACTGCTGATGTTTTCAACAACT
TCAAGGACAAGGAAGGTCGTTTCCGTTCAGAATTGTCTACCGACATCAGAGGTTTGA
TGTCTTTGTACGAAGCTTCTCAATTAAGAATTGAAGGTGAAGACATCTTGGACCAAGC
TGCTGACTTCTCCTCCCAATTGTTGGGTAGATGGACCAAGGATCCAAACCATCACGAA
GCCAGATTGGTTTCTAACACTTTGACTCACCCATACCACAAGTCTTTGGCCACTTTCA
TGGGTCAAAAATTGTCCTATATGAACTGTAAGGGTCCAAACTGGGACGGCGTTGACA
ATTTGCAAGAACTCGCTAAGATGGATTTGACCATTGTTCAATCTATTCACCAAAAGGA
AGTCTTCCAAGTTAGTCAATGGTGGAAAGACACTGGTTTGGCTAATGAATTGAAGCTA
GCCAGAAACCAACCATTGAAGTGGTACATGTGGCCAATGGCTGCCTTGACCGACCCA
AGATTTTCTGAAGAAAGAGTTGAATTGACTAAGCCAATCTCTTTTATCTACATTATTGA
TGATATCTTCGACGTCTACGGTACTATTGAAGAGTTGACTTTGTTCACCGATGCCGTCA
ACAGATGGGAATTGTCTGCTGTTGAACAATTGCCAGACTACATGAAGGTTTGTTTCAA
AGCTTTGTACGACGTCACTAATGAAATTGCTTACAAGATATACAAGAAGCACGGTCAA
AACCCGATTGACTCCTTGCAAAAGACCTGGGCTTCATTGTGTAACGCATTTCTGGTCG
AAGCTAAATGGTTCGCTTCCGGTCATTTGCCAAATGCTGAAGAATACTTGAAGAACG
GTATCATCTCGTCTGGTGTCCACGTTGTTTTGGCTCACATGTTCTTCTTATTAGGTGAC
GGTATCACTCAAGAATCCGTCGATTTGGTCGACGACTACCCAGGTATCTCCACCTCCA
TCGCTACCATCTTGAGATTATCCGACGATCTAGGTTCTGCCAAGGACGAAGACCAAGA
TGGTTACGATGGTTCCTACATCGAATGTTACATGAAAGAACATAAGGGTTCTTCTGTT
GACTCCGCTAGGGAAGAAGTTATCAGAATGATCTCTGAAGCTTGGAAGTGTTTGAAC
AAGGAATGCTTGTCTCCAAACCCATTCTCTGAATCTTTCCGTATTGGTTCGTTAAACAT
GGCTAGAATGATTCCAATGATGTACTCTTACGATGACAACCACAACCTTCCTATCTTGG
AAGAACACATGAAGGCTATGATATACAACACCTCTCTAtaa
The nucleotide sequence of nerolidol synthase from Fragaria x ananassa after
Saccharomyces cerevisiae codon optimization is as follows (5′-3′), and the
gene is named FaNES1.
(SEQ ID NO: 103)
ATGAACGTCGAAACTAAGCACACTAGAACCATGGGTGACATTTTTGTCCAACACTCT
CAAAAGTTGGAACTGTTGAAGACAGTCTTGAGAAATGTTGCTGAATTGGACGCTCTT
GAAGGTTTGAACATGATTGATGCTGTCCAAAGATTAGGTATTGACTACAACTTCCAAA
GAGAAATTGACGAAATCTTACACAAGCAAATGTCCATAGTTTCTGCCAGAGATGACTT
GCACGAAGTCGCTTTGAGATTCAGATTGTTGAGACAACACGGTTACTTCGTTCCAGA
AGATGTTTTCAACAACTTCAAGGACTCCAAGGGTACTTTCAAGCAAGTTTTGGGTGA
AGACATCAAGGGCTTGATGTCTTTATACGAAGCTTCTCAATTGGGAACTGAAGGTGA
AGACATTTTGGTGGAAGCTGAAAAGTTCTCTGGTCACTTGTTAAAGACTTCTTTGTCT
CACTTGGATCATCACAGAGTCAGAATTGTTGCTAACACTTTGCGGAACCCACATCACA
AGTCTTTGGCTCCATTCATGGCTCGTAACTTTTTCGTCACCTCCCAAGCTACCAACTCT
TGGCTAAACTTGTTGAAGGAAGTTGCCAAGACTGATTTCAACATGGTTAGATCTCTCC
ACCAAAATGAAATTGTCCAAATGTCCAAGTGGTGGAAAGAATTGGGTTTAGCAAAGG
AATTAAAATTTGCTAGAGATCAACCATTAAAGTGGTACATCTGGTCTATGGCCTGTTTG
ACTGACCCAAAGTTGTCCGAAGAAAGAGTTGAATTGACCAAGCCAATCAGTTTCGTC
TACTTGATTGACGATATCTTCGATGTCTACGGTACCTTGGATGACTTGATCTTGTTCAC
CGAAGCTGTTAACAGATGGGAAATCACTGCTATCGACCATCTGCCAGACTATATGAAG
ATCTGTTTCAAGGCTTTGTACGACATGACTAACGAATTCTCCTCCAAGGTTTACTTGA
AGCATGGTTGGAACCCATTGCAATCTTTGAAGATTTCCTGGGCTAGCTTGTGCAATGC
TTTCTTGGTTGAGGCCAAATGGTTCGCTTCTGGTAAGCTCCCAAAGTCTGAAGAATAT
CTTAAGAACGGTATCGTCTCTTCCGGTGTCAACGTTGTTTTAGTTCACATGTTCTTTTT
ACTAGGTCAAAACATCACCAGAAAGTCCGTTGAATTGTTGAACGAAACCCCAGCTAT
TATTTCTTCTTCAGCTGCTATTTTGCGTTTGTGGGATGATTTGGGTTCTGCTAAGGATG
AAAACCAAGATGGTAATGACGGTTCCTACGTTAGATGTTACTTGGAAGAACACGAAG
GTTGTTCTATTGAAGAAGCCAGAGAAAAGACCATCAACATGATCTCTGACGAATGGA
AGAAGTTGAACCGTGAATTATTGTCTCCAAACCCTTTCCCAGCCTCCTTCACTTTGGC
TTCATTGAACTTGGCTAGAATGATCCCTTTAATGTACTCTTACGATGGTAACCAATGTT
TGCCATCCCTAAAAGAATACATGAAATTGATGTTGTACGAAACCGTCTCCATGtaa
The nucleotide sequence of nerolidol synthase from Camellia sinensis after
Saccharomyces cerevisiae codon optimization is as follows (5′-3′), and the
gene is named CsNES2.
(SEQ ID NO: 104)
ATGGCCACCACCACTACAACTATCTCAACCACACACAAATGGTCTATCTCTGTTGCTG
AAGACTTGAAGTGTTTGACTCCTCATTGTACTTTCACCAACGAAATCTTAGCTAAGCA
AGAAGAACAATTGGAAGAAGTCAGATCTGTCTTGAGAAAGGTTAGAGAAGAAGAAG
ACTCTCTAAAGCGTTTGGTTATGATCGACGCTTTGCAACGTCTGGGTATTGACTACCA
CTTCAGAGAAGACATCGAAGCTGTTTTGCACTCCCAATACATGAAGTCCTCTTCCCAC
TCTTTCACTTACCAACACGAAGATTTGTACGAAGTTTCCACTAGATTCAGATTGTTGC
GTCAACAAGGCCACAACGTTCCAACTGATGTTTTCGATAAGTTCAAGGACAAGGATG
GTAGATTTAAGACCGAATTGTCTGAAGACATCAGAGGTTTGATGGGTTTATACGAAGC
CTCCCAGTTATCTATTGAAGGTGAAGATATTTTGGACCAAGCTGCCGACTTCTCTGCTC
AACAATTGAACGGTTGGTTGACCAGATTGGGTAACCATCACCAAGCCAAGGTTATCT
CTAACACTCTTGGTCATCCACAACATAAATCATTGGCTAAATTCATGGCTAAGAATTTT
TTGTCTGACTACAACGGTCCAAACGCCAATGTTTTGCAAGAACTAGCGAAGATGGAC
TTCAACATGTCTCCATCCATGTACCAAAAGGAAATGTTGCAAGTCTCTAAGTGGTGGA
AATCCACTGGTTTGGGTAACGAATTAAAGTACGCTAGAAACCAACCATTGAAATGGTA
CTTGTGGTCCATGGCTACTTTAACCCACCCATCTTTGTCTGAACAAAGAGTTGAATTG
ACCAAGCCAATCTCTTTGGTCTACATTATTGACGACATCTTCGACGTTTATGGTACCTT
GGAACAATTAATTTTGTTCACTGAAGCTGTTAACAGATGGGAATTGGCCGCTGTCAAT
GAATTGCCAGGTTACATGAAGATCTGTTTCAAGGTCTTGCAAGATAGTACTAATGAAA
TTGCTTACGGTGTCCACAAGAAGTACGGTTGGAACCCAATTGATAGTTTGAGAAGAA
CCTGGGCTATCTTGTGTAACGCTTTCTTGGAAGAAGCTAAGTGGTTCGCTTCTGGTCA
ATTGCCAAAGGCTGAAGAATACTTAAAGAACGGTATCGTCTCCAGCGGTGTCCCAGC
TGTCCTCGTCCACATGTTTTTCTTGTTGGGTCACGGTATGAACAAGGAATCTGTCGATT
TTACCATCGATAACCCAGACATTATCACTTCTACCGCCACCATTTTGAGATTATGGGAC
GATCTGGGTTCTGCAAAGGATGAAAACCAAGACGGTCACGATGGTTCTTACGTTGAT
TGCTATATGAAAGAAAACGAAGGTTCCACCGCTGAAACTGCTAGAAAGCAAGTTGTT
GACATGATCTCTGATGCTTGGAAGAGATTGAACCGTGAATGTTTGTCTCCAAACAGAT
TCTCCGCTAACTTCACTCAAGGTTGTCTCAACACTGCCAGAATGGTTCCATTGATGTA
CTGTTACGATGACAACCACAACTTGCCTATTTTGGAGGAGCACATGAAGTCCATGTTC
CACGACTCCGTCAGCTTATGA
The nucleotide sequence of nerolidol synthase from Laggera pterodonta after
Saccharomyces cerevisiae codon optimization is as follows (5′-3′), and the
gene is named LpNES1.
(SEQ ID NO: 105)
ATGAAGAAGGAATTATCCACACGTCACTCTGAAGCCTTGGAAGAAGTCAGAAACCTA
TTGAACAATGGTCCATTTAAGAACTTGCACATGGTTGACACTTTGCAAAAGTTGTGTA
TCGACCACCACTTCCAAGAAGAAATTGACATGATTTTCAAGATGTCCAACGTTCATGA
CTACCAAACCCTATACGCCTTATCCTTATATTTCAGAATCTTTAGACAACAAGGTTACT
ACGTTCCAGCTGACGTTTTCGCTAACTTTAAGCAAAAGGATGGTAGATTCAAAGGTG
AAATTGCTCAAGATTTGAAGGGTTTATTAGCTTTATACGAAGCTTCTCAATTATCTATTC
CAGGTGAAGACATTTTGGAGGAAGCTGCTGATTTCTCAAGATACTTGTTGGAACAAA
AGATCCCATTTTTGGGTCAAGAAGAAGCCCTCAAGGTTAACAACACCTTGGAACACT
CCTACCAAAAGATCCCATCTAACTTCAGATTGAAGATCTTCATCAAGCACTACTCTGG
TACCACCATGTCTGACTTGGCTATGCTGGAATTGGCTAAGGCCAAGTCTGTTCACGCT
ACTGAAGTTGAACAATTCTCCAGATGGTGGAAGGATCTAGGTTTGGCTCAAGAATTG
AAATTGGCCAGAAACCAACCATTGCACTGGTACTTGTGGCCAATGGCTGGTTTGACT
GACTTGTCTTTGTCTGAAATCAGAATTGACTTGACCAAGCCAATCGCTTTGGTCTTCA
TTATTGATGACATCTTCGATGTCTACGGTACTTTGGACCAATTGATTTTATTCACTGAA
GCTGTTACCAGATGGGATTCCAATAACTTGGAACAATTGCCATACAATTTGAGAACTT
GTGTTCAAGCTGTCTTTGATGTTACTCACGAAATCTCTGATAAAATTTACAACAAGTA
CGGTTTCAACCCTATTGAATTCTTGAAGCAAACTTGGACTGATTTGTGTGAAGCTTTC
TTAGTCGAAGCAAAGTGGTTCACCAAAGGTTACATGCCAAAGGCTGAAGATTATTTA
AAGAACGGTGCCGTCTCCACCGGTGCTCACGTTGTGATTGTCCACATGTTCTTCTTGT
TGGGTGGTGCCACCTCCAAGGAAACTGCTCAAATCATTGACGAACACAAGGGTATCA
CTTCTTGCTTAGCCAAGATCTTGCGATTGTGGGACGATTTGGGTTCTGCTAAGGACGT
CAACCAAGACGGTCATGACGGCTCATACGTCACTTACTATATGAATGAAAACTCTGGA
TGTTCCATCCAAAACGCTCGTGAACATGTTATGAAGATGATCATCAACACCTGGAAAC
AATTGAACAAAGAATGTTTGTCTCCAAACCCATTCTCTGCCACTTTCACCAAGGCTTG
TTTGAACTTGGCTAGAATGATTCCTTTGATGTACAACTACGATGAAAACCATAGTTTGC
CATTGATCAAGGACTACATTAACTCCATGTTCtaa

Primers were designed to construct above nerolidol synthase genes onto universal vector pZY900. Primer sequences designed for each gene are shown in the table below. Using the synthesized genes above as template, according to the same method as described in 1.2.2 in Example 1, yeast expression vectors including CaNES2, CaNES1, TwNES, AcNES1, FaNES1, CsNES2 and LpNES1 genes were constructed and named pCaNES2, pCaNES1, pTwNES, pAcNES1, pFaNES1, pCsNES2 and pLpNES1, respectively. The plasmid construction schematic diagrams of plasmids pCaNES2, pCaNES1, pTwNES, pAcNES1, pFaNES1, pCsNES2 and pLpNES1 are shown in FIGS. 2G-2M, in which the lac Z gene in pZY900 is replaced by genes CaNES2, CaNES1, TwNES, AcNES1, FaNES1, CsNES2 and LpNES1, respectively.

| Plasmid | Primer | Sequence (5′-3′) |
|---|---|---|
| pCaNES2 | CaNES2-1F | GATATCGACAAAGGAAAAGGGGCCTGTTCATTTGATCA |
| | | AGATACTCTTCAAG (SEQ ID NO: 106) |
| | CaNES2-1R | gaatttttgaaaattcaatataaATGGACTTTTTTGATTGTTCCAG |
| | | (SEQ ID NO: 107) |
| pCaNES1 | CaNES1-1F | GACAAAGGAAAAGGGGCCTGTTCACAAAGCAATGGAC |
| | | AAGATAGAT (SEQ ID NO: 108) |
| | CaNES1-1R | aagaatttttgaaaattcaatataaATGAACTTGCCAATGACTCTAAG |
| | | (SEQ ID NO: 109) |
| pTwNES | TwNES-1F | ATCGACAAAGGAAAAGGGGCCTGTttaCTTGATTAAGAT |
| | | GGATTTCAAGAAT (SEQ ID NO: 110) |
| | TwNES-1R | atttttgaaaattcaatataaATGGCTTTCTTCGGTTCCTCTAG (SEQ |
| | | ID NO: 111) |
| pAcNES1 | AcNES1-1F | CAAAGGAAAAGGGGCCTGTttaTAGAGAGGTGTTGTATA |
| | | TCATAGCC (SEQ ID NO: 112) |
| | AcNES1-1R | gaatttttgaaaattcaatataaATGGCCACTGCCGCTGGTC (SEQ ID |
| | | NO: 113) |
| pFaNES1 | FaNES1-1F | ATCGACAAAGGAAAAGGGGCCTGTttaCATGGAGACGGT |
| | | TTCGTACAAC (SEQ ID NO: 114) |
| | FaNES1-1R | taagaatttttgaaaattcaatataaATGAACGTCGAAACTAAGCACAC |
| | | TAG (SEQ ID NO: 115) |
| pCsNES2 | CsNES2-1F | GACAAAGGAAAAGGGGCCTGTTCATAAGCTGACGGAG |
| | | TCGTGGAAC (SEQ ID NO: 116) |
| | CsNES2-1R | aaagtaagaatttttgaaaattcaatataaATGGCCACCACCACTACAAC |
| | | (SEQ ID NO: 117) |
| pLpNES1 | LpNES1-1F | CGACAAAGGAAAAGGGGCCTGTttaGAACATGGAGTTAA |
| | | TGTAGTCCT (SEQ ID NO: 118) |
| | LpNES1-1R | gtaagaatttttgaaaattcaatataaATGAAGAAGGAATTATCCACAC |
| | | GTC (SEQ ID NO: 119) |

### 1.2 Construction and Shake-Flask Fermentation of Strains Including Nerolidol Synthase Gene From Celastrus angulatus (CaNES2), Celastrus angulatus (CaNES1), Tripterygium wilfordii TwNES (KU588405), Actinidia chinensis (AcNES1), Fragaria x anassa (FaNES1), Camellia sinensis (CsNES2), and Laggera pterodonta (LpNES1)

Plasmid pCaNES2, pCaNES1, pTwNES, pAcNES1, pFaNES1, pCsNES2, and pLpNES1were linearized respectively with MssI, and fragments including the target genes were recovered using a gel recovery kit and introduced into strain JCR27 by lithium acetate method, and the engineered strains were named JCaNES2, JCaNES1, JTwNES, JAcNES1, JFaNES1, JCsNES2, and JLpNES1, respectively. The strains were respectively seeded into YPD liquid medium and incubated under 30° C., 200 rpm with shaking overnight; transferred with an initial OD600=0.1 into 45 ml of YPDHG liquid medium on the next day, followed by incubation for 72 hours under 30° C., 200 rpm shaking, with additional 5 ml isopropyl myristate. The oil layer was collected and the product was detected using GC-MS under the same detecting condition in 1.2.4 of Example 1, the shake flask fermentation yields of strains JCaNES2, JCaNES 1, JTwNES, JAcNES1, JFaNES1, JCsNES2 and JLpNES1 were 33.7 mg/L, 8.3 mg/L, 69 mg/L, 111 mg/L, 86.5 mg/L, 21 mg/Land 5.3 mg/L, respectively.

By comparing Examples 1, 2 and 3 with Comparative Example 1, it can be seen that the nerolidol synthase derived from Tanacetum cinerariifolium, Astilbe chinensis and Artemisia argyi of the present application has better performance than the existing nerolidol synthase.

### Example 4 Construction of A High-Yield Strain Including the Nerolidol Synthase CCJ_TPS23 Derived From Tanacetum cinerariifolium

### 4.1 Construction of High Yield Plasmid

Using the whole genome of Saccharomyces cerevisiae CEN.PK2-1D, plasmid pRS423, the whole genome of Saccharomyces cerevisiae CEN.PK2-1D, the whole genome of Saccharomyces cerevisiae S288C, the whole genome of Saccharomyces cerevisiae CEN.PK2-1D, Tanacetum cinerariifolium cDNA, the whole genome of CEN.PK2-1D, the whole genome of CEN.PK2-1D, and pRS426 plasmid as templates, amplify with primers 020-1F/R, 020-2F/R, 020-3F/R, 020-4F/R, 020-5F/R, 020-6F/R, 020-7F/R, 020-8F/R and 020-9F/R, respectively, to obtain the fragments which are left homologous arm (HA) of Ura3, histidine screening marker (HIS3), CYC1 terminator (T), tHMG1 gene, GAL1-GAL10 promoters (P_{GAL10} and P_{GAL1}), CCJ_TPS23, PGK1 terminator (T), right homologous arm (HA) of Ura3, and plasmid backbone. Then, the plasmid pYR020 was obtained by recombinant construction in Saccharomyces cerevisiae using the same yeast assembly method as in Example 1, and sequentially ligating the above fragments. The plasmid construction schematic diagram of plasmid pYR020 is shown in FIG. 9A as pYR020, which also includes the MssI restriction site, and the rest part derives from the pRS426 plasmid backbone.

Primer sequences are shown in Table 5 below.

TABLE 5

| Primer | Sequence (5′-3′) |
|---|---|
| 020-1F | attaaccctcactaaagggaacaaaagcgtttaaacacgcagataattccaggtatttt (SEQ ID NO: 45) |
| 020-1R | aatacgactcactatagggcgaattgggtaccttcgtttcctgcaggtttttgt (SEQ ID NO: 46) |
| 020-2F | caaaaacctgcaggaaacgaaggtacccaattcgccctatagtgag (SEQ ID NO: 47) |
| 020-2R | gttttgggacgctcgaaggctttaatttgctcacagcttgtctgtaagcg (SEQ ID NO: 48) |
| 020-3F | ttgtctgctcccggcatccgcttacagacaagctgtgagcaaattaaagccttcgagcg (SEQ ID NO: 49) |
| 020-3R | gtttgaaagatgggtccgtcacctgcattaaatcctaaacaggccccttttcctttgtc (SEQ ID NO: 50) |
| 020-4F | taattacatgatatcgacaaaggaaaaggggcctgtttaggatttaatgcaggtgacgg (SEQ ID NO: 51) |
| 020-4R | gaatttttgaaaattcaatataaatggttttaaccaataaaacagtcat (SEQ ID NO: 52) |
| 020-5F | gttttattggttaaaaccatttatattgaattttcaaaaattcttactttttttttgg (SEQ ID NO: 53) |
| 020-5R | taacacaattgttgatgatcattatagttttttctccttgacgttaaagt (SEQ ID NO: 54) |
| 020-6F | gtcaaggagaaaaaactataatgatcatcaacaattgtgttacac (SEQ ID NO: 55) |
| 020-6R | cgatttcaattcaattcaatttaaaacatggagtttatatattccttgatg (SEQ ID NO: 56) |
| 020-7F | tatataaactccatgttttaaattgaattgaattgaaatcgatagatcaat (SEQ ID NO: 57) |
| 020-7R | ttgaagctctaatttgtgagtttagtatacatgcatttacaacgaacgcagaattttcg (SEQ ID NO: 58) |
| 020-8F | gtttaataactcgaaaattctgcgttcgttgtaaatgcatgtatactaaactcacaaat (SEQ ID NO: 59) |
| 020-8R | gacggtcacagcttgtctgtgtttaaaccgtttaagggcaaatgtactct (SEQ ID NO: 60) |
| 020-9F | agagtacatttgcccttaaacggtttaaacacagacaagctgtgaccgtc (SEQ ID NO: 61) |
| 020-9R | tgcttcaaaatacctggaattatctgcgtgtttaaacgcttttgttccctttagtgagg (SEQ ID NO: 62) |

Using the whole genome of Saccharomyces cerevisiae CEN.PK2-1D, plasmid pRS424, the whole genome of Saccharomyces cerevisiae S288C, the whole genome of Saccharomyces cerevisiae CEN.PK2-1D, Tanacetum cinerariifolium cDNA, the whole genome of Saccharomyces cerevisiae CEN.PK2-1D, the whole genome of Saccharomyces cerevisiae CEN.PK2-1D, and pRS426 plasmid as templates, amplify with primers 021-1F/R, 021-2F/R, 021-3F/R, 021-4F/R, 021-5F/R, 021-6F/R, 021-7F/R and 021-8F/R, respectively, to obtain the fragments which are left homologous arm (HA) of YPRCdelta15, tryptophan selection marker (TRP1), GPM1 terminator (T), GAL1-GAL10 promoter (P_{GAL10} and P_{GAL1}), CCJ_TPS23, PGK1 terminator (T), right homologous arm (HA) of YPRCdelta15, and plasmid backbone. Then, the plasmid pYR021 was obtained by recombinant construction in Saccharomyces cerevisiae using the same yeast assembly method as aforementioned, and sequentially ligating the above fragments. The plasmid construction schematic diagram of plasmid pYR021 is shown in FIG. 9C as pYR021, which also includes the Not I restriction site, and the rest part derives from the pRS426 plasmid backbone.

Primer sequences are shown in Table 6 below.

TABLE 6

| Primer | Sequence (5′-3′) |
|---|---|
| 021-1F | ctaaagggaacaaaagcgcggccgcggcaatttggtacaaaaatcacg (SEQ ID NO: 63) |
| 021-1R | ctatattatatatatagtaatgtcgtttttgcgaaaccctatgctc (SEQ ID NO: 64) |
| 021-2F | gagcatagggtttcgcaaaaacgacattactatatatataatatag (SEQ ID NO: 65) |
| 021-2R | gctgaatgggcagttcgaatacctgatgcggtattttctcc (SEQ ID NO: 66) |
| 021-3F | ggagaaaataccgcatcaggtattcgaactgcccattcagc (SEQ ID NO: 67) |
| 021-3R | gtaagaatttttgaaaattcaatataagtctgaagaatgaatgatttgatgat (SEQ ID NO: 68) |
| 021-4F | aatcattcattcttcagacttatattgaattttcaaaaattcttactttttttttg (SEQ ID NO: 69) |
| 021-4R | aacacaattgttgatgatcattatagttttttctccttgacg (SEQ ID NO: 70) |
| 021-5F | gtcaaggagaaaaaactataatgatcatcaacaattgtgttacac (SEQ ID NO: 71) |
| 021-5R | cgatttcaattcaattcaatttaaaacatggagtttatatattccttgatg (SEQ ID NO: 72) |
| 021-6F | tatataaactccatgttttaaattgaattgaattgaaatcg (SEQ ID NO: 73) |
| 021-6R | gctcatcccgaccttccattaacgaacgcagaattttcgag (SEQ ID NO: 74) |
| 021-7F | ctcgaaaattctgcgttcgttaatggaaggtcgggatgagc (SEQ ID NO: 75) |
| 021-7R | agacggtcacagcttgtctgtgcggccgcgcttctaataaaccgatgaacgc (SEQ ID NO: 76) |
| 021-8F | gcgttcatcggtttattagaagcgcggccgcacagacaagctgtgaccgtct (SEQ ID NO: 77) |
| 021-8R | ttttgtaccaaattgccgcggccgcgcttttgttccctttagtgagg (SEQ ID NO: 78) |

### 4.2 Construction of Gene Editing Plasmids and Knockout Cassettes

Using plasmid pKIURA100 (see literature Zhang, Yueping et al. “A gRNA-tRNA array for CRISPR-Cas9 based rapid multiplexed genome editing in Saccharomyces cerevisiae.” Nature communications vol. 10,1 1053. 5 Mar. 2019, doi:10.1038/s41467-019-09005-3) as the template, amplify with primers 3951-F/R to obtain fragments. Goldengate method (Zhang, Yueping et al. “A gRNA-tRNA array for CRISPR-Cas9 based rapid multiplexed genome editing in Saccharomyces cerevisiae.” Nature communications vol. 10,1 1053. 5 Mar. 2019, doi:10.1038/s41467-019-09005-3) was then used to assemble the above fragments and pCas to construct plasmid pYH395, and the plasmid construction schematic diagram is shown in FIG. 10A.

Primer sequences are shown in Table 7 below.

TABLE 7

| Primer | Sequence (5′-3′) |
|---|---|
| 3951-F | aaaggtctcagatcttttccactgcactttgcatgttttagagctagaaatagcaagtt (SEQ ID NO: 79) |
| 3951-R | aaaggtctcaaaactctagactttttcgatgatgtagtttct (SEQ ID NO: 80) |

Using the whole genome of Saccharomyces cerevisiae CEN.PK2-1D as the template, amplify with primers ERG9-1F/R and ERG9-2F/R to obtain two fragments, and then overlap extension PCR was performed with the obtained two fragments as the template to obtain ERG9 knockout cassette. A schematic diagram of the knockout element construction of the upstream activation cis element (−220 to −175) of the ERG9 promoter is shown in FIG. 10B, wherein the two HAs represent the homology arm to the left of the sequence (−220 to −175) and the homology arm to the right of the sequence (−220 to −175), respectively.

Primer sequences are shown in Table 8 below.

TABLE 8

| Primer | Sequence (5′-3′) |
|---|---|
| ERG9-1F | atagaagacgaacattgtacgatac (SEQ ID NO: 81) |
| ERG9-1R | acccaaaaccgataacgccttccgataagtcggtattgttgttgaagatg (SEQ ID NO: 82) |
| ERG9-2F | tcaacaacaataccgacttatcggaagggttatcggttttgg (SEQ ID NO: 83) |
| ERG9-2R | aatttctcgtggaagtgacg |
| | (SEQ ID NO: 84) |

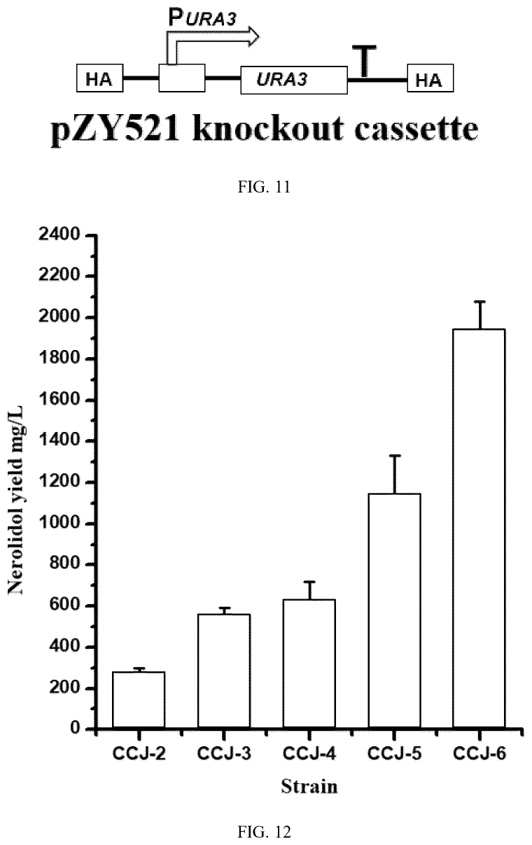

Using the whole genome of Saccharomyces cerevisiae CEN.PK2-1D, pRS426 plasmid and the whole genome of Saccharomyces cerevisiae CEN.PK2-1D as templates, amplify with primers 5211-F/R, 5212-F/R and 5213-F/R to obtain three fragments, and then overlap extension PCR was performed by using primers 5211-F and 5213-R with the obtained three fragments as templates to obtain pZY521 knockout cassette. The schematic diagram of the construction of pZY521 knockout cassette is shown in FIG. 11, in which the sequences are sequentially GAL80 left homology arm (HA), URA3 screening marker (promoter PURA3, URA3 gene, terminator T) and GAL80 right homology arm (HA).

Primer sequences are shown in Table 9 below.

TABLE 9

| Primer | Sequence (5′-3′) |
|---|---|
| 5211 F | caatggtctaggtagtggcattcg (SEQ ID NO: 85) |
| 5211-R | cgactcactatagggcgaattgggtacgacgggagtggaaagaacgg (SEQ ID NO: 86) |
| 5212-F | tcccgttctttccactcccgtcgtacccaattcgccctatagtgag (SEQ ID NO: 87) |
| 5212-R | gccaagcacagggcaagatgctttcacagcttgtctgtaagcgga (SEQ ID NO: 88) |
| 5213-F | gcatccgcttacagacaagctgtgaaagcatcttgccctgtgctt (SEQ ID NO: 89) |
| 5213-R | gattccatgctaccttccatggttg |
| | (SEQ ID NO: 90) |

### 4.3 Strain Construction

1) Plasmid pYR013 was digested with MssI (see the instruction manual of MssI enzyme for conditions of enzyme digestion system) to obtain linearized fragments, which were integrated into strain JCR27 by lithium acetate method to obtain strain CCJ-2.
2) Plasmid pYR020 was linearized with MssI enzyme and integrated into strain CCJ-2 in the same way as in step 1) to obtain strain CCJ-3.
3) Plasmid pYR021 was linearized with NotI enzyme and integrated into strain CCJ-3 in the same way as in step 1) to obtain strain CCJ-4.
4) Plasmid pYH395 and ERG9 knockout cassette were co-transferred into CCJ-4 by lithium acetate method, and the correctness of the knockout for the colonies were verified by PCR, before incubated in YPD liquid medium (20 g/L tryptone, 10g/L yeast extract,20 g/L glucose), with shaking at 220 rpm, at 30° C. for 8 hours, and then washed with water. After being streaked on 5-FOA plate (Xiaowei Li. Engineering Acetyl-CoA Pathway to Construct Efficient Biosynthesis Platform of Saccharomyces Cerevisiae [D]. Wuhan University, 2015.) and cultured in incubator at 30° C. for 3 days, the strains were picked from the plate. The correct strain was named CCJ-5 after the correctness of the knockout for the colonies being verified by PCR.

This step is used to knock out the upstream activation cis element (-220 to-175) of ERG9 promoter, squalene synthase encoded by ERG9 synthesizes squalene by using FPP as the substrate, which is a competitive pathway of nerolidol synthesis pathway, and the knocking out of the upstream activation cis element of ERG9 promoter can downregulate the squalene synthesis pathway, reduce the competition of squalene synthesis pathway to consume the substrate required for nerolidol synthesis, and further improve the yield of nerolidol.

5) pZY521 knockout cassette was transferred into CCJ-5 by lithium acetate method to obtain CCJ-6. The step has the effect of knocking out transcription inhibitor GAL80, so that the target gene in the transferred bacteria can be expressed autonomously without an inducer, which is beneficial to reducing the cost of fermentation and further improving the yield of nerolidol.

### 4.4 Shake Flask Fermentation of the Strain

The strains CCJ-2, CCJ-3, CCJ-4, CCJ-5 and CCJ-6 were seeded respectively into YPD liquid medium and incubated under 30° C., 200 rpm with shaking overnight; transferred with an initial OD600=0.1 into 45 ml of YPDHG liquid medium (CCJ-2, CCJ-3, CCJ-4, and CCJ-5) or YPD liquid medium (CCJ-6) on the next day, followed by incubation for 72 hours under 30° C., 200 rpm shaking, with additional 5 ml isopropyl myristate. The oil layer was collected and the product was detected using GC-MS, under the same conditions as in 1.2.4 of Example 1. Nerolidol yield data for different strains are shown in FIG. 12. The results showed that the yield of nerolidol increased gradually with the increase of the copy number of the subject gene CCJ_TPS23, 271 mg/L for CCJ-2, 557 mg/L for CCJ-3, and 627 mg/L for CCJ-3. After downregulating of the squalene synthesis pathway (CCJ-5), nerolidol yield further increased significantly and reached 1140 mg/L. Further knockout of galactose-induced transcription inhibitor GAL80 (CJ-6) leads to nerolidol yield further increased significantly and reached 1942 mg/L.

### Example 5 Construction of a High-Yield Strain Including Nerolidol Synthetase ACH_TPS07 Derived From Astilbe chinensis 5.1 Construction of High Yield Plasmid

Using the whole genome of Saccharomyces cerevisiae CEN.PK2-1D, plasmid pRS423, the whole genome of Saccharomyces cerevisiae CEN.PK2-1D, the whole genome of Saccharomyces cerevisiae S288C, the whole genome of Saccharomyces cerevisiae CEN.PK2-1D, Astilbe chinensis cDNA, the whole genome of CEN.PK2-1D, the whole genome of CEN.PK2-1D, and pRS426 plasmid as templates, amplify with primers 020-1F/R, 020-2F/R, 020-3F/R, 020-4F/R, 020-5F/017-5R, 017-6F/R, 017-7F/020-7R, 020-8F/R and 020-9F/R, respectively, to obtain the fragments which are left homologous arm (HA) of Ura3, histidine screening marker (HIS3), CYC1 terminator (T), tHMG1 gene, GAL1-GAL10 promoters (P_{GAL10} and P_{GAL1}), ACH_TPS07, PGK1 terminator (T), right homologous arm (HA) of Ura3, and plasmid backbone. Then, the plasmid pYR017 was obtained by recombinant construction in Saccharomyces cerevisiae using the same yeast assembly method as in Example 1, and sequentially ligating the above fragments. The plasmid construction schematic diagram of plasmid pYR017 is shown in FIG. 9B as pYR017, which also includes the MssI restriction site, and the rest part derives from the pRS426 plasmid backbone.

Primer sequences are shown in Table 10 below.

TABLE 10

| Primer | Sequence (5′-3′) |
|---|---|
| 017-5R | aggaggaaggagggggtgccattatagttttttctccttgacgttaaagt (SEQ ID NO: 91) |
| 017-6F | gtcaaggagaaaaaactataatggcaccccctccttcc (SEQ ID NO: 92) |
| 017-6R | cgatttcaattcaattcaatttaaaacaaacattgtatatgctcctcaag (SEQ ID NO: 93) |
| 017-7F | catatacaatgtttgttttaaattgaattgaattgaaatcgatagatcaat (SEQ ID NO: 94) |

Using the whole genome of Saccharomyces cerevisiae CEN.PK2-1D, plasmid pRS424, the whole genome of Saccharomyces cerevisiae S288C, the whole genome of Saccharomyces cerevisiae CEN.PK2-1D, Astilbe chinensis cDNA, the whole genome of Saccharomyces cerevisiae CEN.PK2-1D, the whole genome of Saccharomyces cerevisiae CEN.PK2-1D, and pRS426 plasmid as templates, amplify with primers 021-1F/R, 021-2F/R, 021-3F/R, 021-4F/018-4R, 018-5F/R, 018-6F/021-6R, 021-7F/R and 021-8F/R, respectively, to obtain the fragments which are left homologous arm (HA) of YPRCdelta15, tryptophan selection marker (TRP1), GPM1 terminator (T), GAL1-GAL10 promoter (P_{GAL10} and P_{GAL1}), ACH_TPS07, PGK1 terminator (T), right homologous arm (HA) of YPRCdelta15, and plasmid backbone. Then, the plasmid pYR018 was obtained by recombinant construction in Saccharomyces cerevisiae using the same yeast assembly method as aforementioned, and sequentially ligating the above fragments. The plasmid construction schematic diagram of plasmid pYR018 is shown in FIG. 9D as pYR018, which also includes the Not I restriction site, and the rest part derives from the pRS426 plasmid backbone.

Primer sequences are shown in Table 11 below.

TABLE 11

| Primer | Sequence (5′-3′) |
|---|---|
| 018-4R | ggaggaaggagggggtgccattatagttttttctccttgacg |
| | (SEQ ID NO: 95) |
| 018-5F | gtcaaggagaaaaaactataatggcaccccctccttcc (SEQ ID NO: 96) |
| 018-5R | cgatttcaattcaattcaatttaaaacaaacattgtatatgctcctc (SEQ ID NO: 97) |
| 018-6F | catatacaatgtttgttttaaattgaattgaattgaaatcg (SEQ ID NO: 98) |

### 5.2 Strain Construction

Plasmid pYR007 was linearized with MssI and integrated into strain JCR27 to obtain strain LXF-2, plasmid pYR017 was linearized with MssI and integrated into strain LXF-2 to obtain strain LXF-3, plasmid pYR018 was linearized with NotI and integrated into strain LXF-3 to obtain strain LXF-4, using the same method as in Example 4. Plasmid pYH395 and ERG9 knockout cassette were co-transferred into strain LXF-4, and the correctness of the knockout for the colonies were verified by PCR, before incubated in YPD liquid medium, and then washed with water. After being streaked on 5-FOA plate, the strains were picked from the plate. The correct strain was named LXF-5 after the correctness of the knockout for the colonies being verified by PCR; pZY521 knockout cassette was transferred into strain LXF-5 to obtain strain LXF-6.

### 5.3 Shake Flask Fermentation of the Strain

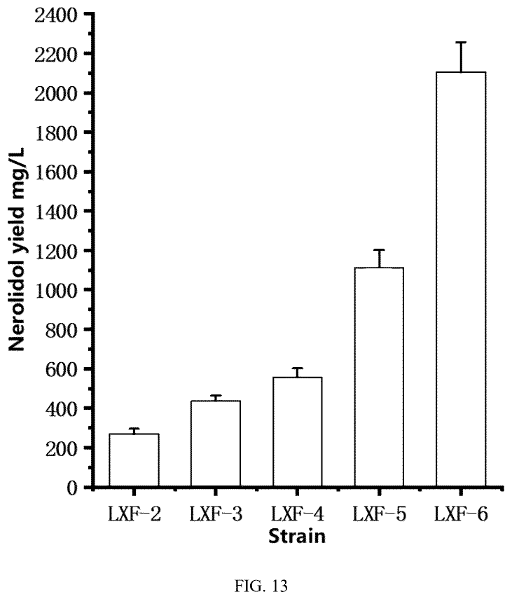

The strains LXF-2, LXF-3, LXF-4, LXF-5, and LXF-6 were seeded respectively into YPD liquid medium and incubated under 30° C., 200 rpm with shaking overnight; transferred with an initial OD600-0.1 into 45 ml of YPDHG liquid medium ((LXF-2, LXF-3, LXF-4, and LXF-5) or YPD liquid medium (LXF-6) on the next day, followed by incubation for 72 hours under 30° C., 200 rpm shaking, with additional 5 ml isopropyl myristate. The oil layer was collected and the product was detected using GC-MS, under the same conditions as in 1.2.4 of Example 1. The yield data are shown in FIG. 13. Nerolidol yield data for different strains are shown in FIG. 13. The results showed that the yield of nerolidol increased gradually with the increase of the copy number of the subject gene ACH_TPS07, about 269 mg/L for LXF-2, about 436 mg/L for LXF-3, and about 556 mg/L for LXF-4. After downregulating of the squalene synthesis pathway (LXF-5), nerolidol yield further increased significantly and reached 1112 mg/L. Further knockout of transcription inhibitor GAL80 (LXF-6) leads to nerolidol yield further increased significantly and reached 2105 mg/L.

### Example 6 Fermentor Fermentation of Nerolidol High-Yield Strain

According to the fermentor medium and fermentation method described in literature (Ye Z, Huang Y, Shi B, et al. Coupling cell growth and biochemical pathway induction in Saccharomyces cerevisiae for production of (+)-valencene and its chemical conversion to (+)-nootkatone [published online ahead of print, 2022 Mar. 13]. Metab Eng. 2022;72:107-115. doi:10.1016/j.ymben. 2022.03.005), the constructed strain LXF-6 was subjected to fed-batch fermentation, and a covering agent was added in the fermentation process to achieve in situ extraction, and the covering agent was isopropyl myristate. The fermentation process was controlled so that the dissolved oxygen is more than 20%, pH is 5, glucose concentration is 1-2g/L, and ethanol concentration is less than 5g/L. Eventually, the yield of nerolidol reached 55.7 g/L in 1 L fermentor, which is the highest level reported so far.

What is claimed is:

1. A nerolidol synthase comprising domains of Pfam Nos. PF01397 and PF03936, and having an activity of the nerolidol synthase.

2. The nerolidol synthase according to claim 1, wherein the nerolidol synthase is derived from Tanacetum cinerariifolium, Astilbe chinensis, or Artemisia argyi

3. The nerolidol synthase according to claim 1, comprising an amino acid sequence having at least 90%, 91%, 92%, 93%, 94%, 95%, 96%, 97%, 98%, 99%, or 100% sequence identity to the amino acid sequence as shown in SEQ ID NO: 1, SEQ ID NO: 2, SEQ ID NO: 3, SEQ ID NO: 4, SEQ ID NO: 11, or SEQ ID NO: 12.

4. A polynucleotide molecule comprising at least one of nucleotide sequences encoding the nerolidol synthase according to claim 1 or a complementary sequence thereof.

5. The polynucleotide molecule according to claim 4, comprising a nucleotide sequence having at least 90%, 91%, 92%, 93%, 94%, 95%, 96%, 97%, 98%, 99%, or 100% sequence identity to the nucleotide sequence as shown in SEQ ID NO: 5, SEQ ID NO: 6, SEQ ID NO: 7, SEQ ID NO: 8, SEQ ID NO: 9, or SEQ ID NO: 10.

6. A nucleic acid construct comprising the polynucleotide molecule according to claim 4.

7. The nucleic acid construct according to claim 6, further comprising at least one of nucleotide sequences encoding acetoacetyl coenzyme A thiolase, hydroxy-methylglutaryl coenzyme A synthase, hydroxy-methylglutaryl coenzyme A reductase, mevalonate kinase, mevalonate-5-phosphate kinase, mevalonate pyrophosphate decarboxylase, isoprene pyrophosphate isomerase, and farnesyl pyrophosphate synthase.

8. The nucleic acid construct according to claim 6, wherein the at least one of nucleotide sequences encoding the nerolidol synthase is located between two insertion elements, wherein the two insertion elements are configured for integrating the at least one of nucleotide sequences encoding the nerolidol synthase into a genome of a host cell.

9. The nucleic acid construct according to claim 6, wherein the nucleic acid construct is a plasmid vector; preferably, the plasmid vector is a eukaryotic expression vector.

10. The nucleic acid construct according to claim 9, further comprising a pRS426 plasmid backbone.

11. The nucleic acid construct according to claim 10, wherein the nucleic acid construct is at least one of plasmid vectors pYR006, pYR007, pYR010, pYR013, pAra-TPS27, pAra-TPS28, pYR017, pYR018, pYR020, and pYR021, wherein a construction schematic diagram of the plasmid vectors is as shown in FIGS. 2A-2F or FIGS. 9A-9D.

12. A recombinant strain comprising the polynucleotide molecule according to claim 4, or a nucleic acid construct comprising the polynucleotide molecule.

13. The recombinant strain according to claim 12, wherein the polynucleotide molecule is integrated into a genome of a host cell; preferably, the host cell is a eukaryotic cell, more preferably is Saccharomyces cerevisiae.

14. The recombinant strain according to claim 13, wherein a copy number of the polynucleotide molecule in a genome of the recombinant strain is 1-3.

15. The recombinant strain according to claim 12, wherein the recombinant strain is configured for endogenously and/or exogenously expressing at least one of acetoacetyl coenzyme A thiolase, hydroxy-methylglutaryl-CoA synthase, hydroxy-methylglutaryl-CoA reductase, mevalonate kinase, mevalonate-5-phosphate kinase, mevalonate pyrophosphate decarboxylase, isoprene pyrophosphate isomerase, and farnesyl pyrophosphate synthase.

16. The recombinant strain according to claim 15, wherein copy numbers of nucleotide sequences encoding the acetoacetyl coenzyme A thiolase, the hydroxy-methylglutaryl-CoA synthase, the hydroxy-methylglutaryl-CoA reductase, the mevalonate kinase, mevalonate-5-phosphate kinase, the mevalonate pyrophosphate decarboxylase, the isoprene pyrophosphate isomerase, and the farnesyl pyrophosphate synthase in a genome of the recombinant strain are 2, 2, 4, 2, 2, 2, 2, and 2, respectively.

17. The recombinant strain according to claim 12, further comprising downregulation or knockout of at least one of genes encoding FPP hydrolase DPP1, FPP hydrolase LPP1, citrate synthase, malate synthase, or squalene synthase.

18. A method for producing nerolidol, comprising using the nerolidol synthase according to claim 1, a polynucleotide molecule comprising at least one of nucleotide sequences encoding the nerolidol synthase or a complementary sequence thereof, a nucleic acid construct comprising the polynucleotide molecule, or a recombinant strain comprising the polynucleotide molecule or the nucleic acid construct.

19. A method for preparing nerolidol, comprising biosynthesizing the nerolidol by using the recombinant strain according to claim 12.

20. The polynucleotide molecule according to claim 4, wherein the nerolidol synthase is derived from Tanacetum cinerariifolium, Astilbe chinensis, or Artemisia argyi.
